(19) United States
(12) Patent Application Publication
(10) Pub. No.: US 2011/0314573 A1
(43) Pub. Date: Dec. 22, 2011

(54) SCREENING METHOD FOR IDENTIFYING GENES INVOLVED IN PLANT CELL CYCLE

(76) Inventors: Geert De Jaeger, Evergem (BE); Nathalie Gonzalez, Merelbeke (BE); Dirk G. Inze, Moorsel-Aalst (BE); Jelle Van Leene, Gent (BE); Henri Van Onckelen, Antwerpen (BE); Erwin Witters, Wilrijk (BE)

(21) Appl. No.: 13/139,268

(22) PCT Filed: Dec. 10, 2009
(86) PCT No.: PCT/EP09/66856

§ 371 Date: Aug. 31, 2011

(30) Foreign Application Priority Data
Dec. 10, 2008 (EP) 08171187.1

(51) Int. Cl. C12N 15/82; G01N 33/566; A01P 21/00; A01N 37/18; C07K 14/00

(52) U.S. Cl. 800/290; 504/335; 530/350; 436/501

(57) ABSTRACT

The present invention relates to a method for screening proteins related to and/or involved in plant cell cycle. It further relates to proteins isolated with the method and the use of those proteins and/or the genes encoding those proteins for modulating plant yield and plant growth.

### CROSS-REFERENCE TO RELATED APPLICATION(S)

This is a national phase entry under 35 U.S.C. §371 of International Patent Application PCT/EP2009/066856, filed Dec. 10, 2009, published in English as International Patent Publication WO 2010/066849 A1 on Jun. 17, 2010, which claims the benefit under Article 8 of the Patent Cooperation Treaty to European Patent Application Serial No. 08171187.1, filed Dec. 10, 2008.

### TECHNICAL FIELD

The present invention relates to a novel method for screening proteins related to and/or involved in plant cell cycles. It further relates to proteins isolated with the method and the use of those proteins and/or the genes encoding those proteins for modulating plant yield and plant growth.

### BACKGROUND

Knowledge of the basic cell cycle machinery is a prerequisite to grasp how signaling pathways impinge on and regulate cell proliferation during plant growth and development in a changing environment. The fundamental underlying mechanisms of cell division are conserved among all eukaryotes, however, due to their sessile lifestyle, plants have evolved unique features. Plant genome sequence analysis revealed the existence of an unexpected high number of genes involved in cell proliferation (Capron et al., 2003; Vandepoele et al., 2002; Menges et al., 2005; Schultz et al., 2007), compared to other organisms. Microarray analysis showed that a lot of these follow a cell cycle-dependent expression profile (Menges et al., 2005), sustaining their role in cell cycle regulation.

### SUMMARY OF THE INVENTION

To elucidate which molecular machines are involved in plant cell division, we isolated cellular complexes by TAP from Arabidopsis thaliana cell suspension cultures (Van Leene et al., 2007; Van Leene et al., 2008) using “core” cell cycle proteins as baits. The dataset was corrected for non-specific interactions and divided in a “core” dataset of interactions that were biologically confirmed in at least two independent repeats, and a “non-core” dataset. Surprisingly, we found that the datasets, apart from known cell cycle proteins, were also comprising proteins of which the role in cell cycle never has been illustrated before.

The robustness of both the core and non-core datasets is demonstrated through different computational analysis and through the biological interpretation of the network. The interactome serves as an excellent hypothesis-generating tool, and the power of it is reached in particular when integrated with other data. Combining our interactome data with cell cycle-related expression profiles, for example, gives insight in which CDK/cyclin complexes are active during cell division and when. The data prove that the high numbers of cell cycle regulators in plants are not the sole consequence of redundancy, but that different cell cycle regulators are combined in complexes providing functional diversity leading to increased complexity and flexibility of the plant cell cycle.

Disclosed herein is a method to isolate novel cell cycle-related proteins, comprising (1) performing a tap analysis using a known cell cycle protein as bait, (2) correcting the results for non-specific interactions, and (3) reconfirming the corrected results. “Reconfirming the result,” as used here, can be done either by repeating the tap-tag experiment or by carrying out a reversed tap-tag, wherein the original prey is now used as bait.

Also disclosed herein is the use of a cell cycle-related protein isolated with the method of the invention for the modulation of plant growth and/or yield. In a particular embodiment, the plant is a crop plant, preferably a monocot or a cereal, even more preferably it is a cereal selected from the group consisting of rice, maize, wheat, barley, millet, rye, sorghum and oats.

The use, as indicated here, is the use of the protein and/or the use of a nucleic acid encoding this protein, or the complement thereof It includes, but is not limited to, genomic DNA, cDNA, messenger RNA (including the 5′ and 3′ untranslated regions) and RNAi; the use can result, as a non-limiting example, in overexpression or repression of the expression of the gene. Overexpression or repression of expression of a target gene can be obtained by transfer of a genetic construct, intended for the overexpression or the repression of expression into a plant. The transfer of foreign genes into the genome of a plant is called transformation. Transformation of plant species is a fairly routine technique known to the person skilled in the art. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include, but are not limited to, agrobacterium-mediated transformation, the use of liposomes, electroporation, chemicals that increase free DNA uptake, injection of the DNA directly into the plant, particle gun bombardment, transformation using viruses or pollen and microprojection.

Preferably, the cell cycle-related proteins are selected from the group consisting of At1g56110, At3g17020, At3g21140, At5g25460, At5g60790, At4g38900, At3g49240, At5g24690, At1g06070, At4g34150, At1g20480, At5g20920, At3g15970, At5g13030, At1g01880, At5g07310, At2g46610, At1g10690, At3g04710, At3g24690, At4g16130, At2g05830, At1g29220, At1g55890, At1g60650, At1g70830, At2g43140, At1g77180, At5g18620, At5g02530, At5g14170, At1g52730, At2g33340, At1g03060, At3g62240, At4g38740, At5g61220, At3g53880, At3g56860, At1g01970, At1g19520, At1g14620, At2g03820, At3g01280, At3g56690, At5g41190, At5g03740, At1g42440, At2g28450, At1g09760, At1g10840, At3g11830, At5g54900, At1g31760, At1g61870, At3g11760, At1g05805, At1g29200, At4g13850, At4g38780, At1g71380, At3g13640, At5g25060, At1g43700, At2g46020, At3g55760 and At5g21160 or a variant thereof. “Variants,” as used here, are including, but not limited to, homologues, orthologues and paralogues of the cell cycle-related proteins. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. Preferably, the homologue, orthologue or paralogue has a sequence identity at a protein level of at least 50%, 51%, 52%, 53%, 54% or 55%, 56%, 57%, 58%, 59%, preferably 60%, 61%, 62%, 63%, 64%, 65%, 66%, 67%, 68%, 69%, more preferably 70%, 71%, 72%, 73%, 74%, 75%, 76%, 77%, 78%, 79%, even more preferably 80%, 81%, 82%, 83%, 84%, 85%, 86%, 87%, 88%, 89%, and most preferably 90%, 91%, 92%, 93%, 94%, 95%, 96%, 97%, 98%, 99% or more as measured in a BLASTp (Altschul et al., 1997; Altschul et al., 2005).

Preferably, the use is overexpression of the gene encoding the cell cycle-related protein, even more preferably it is overexpression of a cell cycle-related protein according to the invention, selected from the group consisting of At1g56110, At3g17020, At3g21140, At5g25460, At5g60790 (SEQ ID NOS:1-5), or a variant thereof. Preferably, overexpression results in an increase of plant growth and/or yield. Increase of plant growth and/or yield is measured by comparing the test plant, comprising a gene used according to the invention, with the parental, non-transformed plant, grown under the same conditions as control. Preferably, increase of growth is measured as an increase of biomass production. “Yield” refers to a situation where only a part of the plant, preferably an economically important part of the plant, such as the leaves, roots or seeds, is increased in biomass. The term “increase” as used here means at least a 5%, 6%, 7%, 8%, 9% or 10%, preferably at least 15% or 20%, more preferably 25%, 30%, 35% or 40% more yield and/or growth in comparison to control plants as defined herein. “Increase of plant growth,” as used here, is preferably measured as increase of any one or more of total plant biomass, leaf biomass, root biomass and seed biomass. In one preferred embodiment, the increase is an increase in total plant biomass. In a preferred embodiment, the plant is a crop plant, preferably a monocot or a cereal; even more preferably, it is a cereal selected from the group consisting of rice, maize, wheat, barley, millet, rye, sorghum and oats.

Still another aspect of the invention is a novel cell cycle-related protein, isolated with the method according to the invention.

### BRIEF DESCRIPTION OF THE FIGURES

FIG. 1: Panel A: Enrichment analysis on the core and non-core datasets. Statistically significant enrichments are indicated with * (p-value<0.05) or with ** (p-value<0.01). An enrichment analysis was performed for genes showing cell cycle-regulated expression (Periodic), for genes containing an E2F or MSA consensus motif in their promoter and for proteins containing a CDK consensus phosphorylation site. Panel B: Percentage of the whole gene pool (genome-wide), a cell cycle collection of 518 genes, bait proteins (without reverse baits), and core and non-core preys (without the cell cycle collection) having at least two features. P-values are shown in red. Panel C: Distribution of the transcript PCC of the core (red curve) and non-core dataset (green curve), compared to the average distribution (black and grey curves) of 100 random networks containing an equal number of interactions and genes.

FIG. 2: Distribution of the number of cell cycle-related features among the whole gene pool (genome-wide), a collection of 518 cell cycle genes, and bait proteins (without reverse baits).

FIG. 3: Overview of the subnetworks.

FIG. 4: Fresh weight of control lines transformed with empty vector. About 100 plants were analyzed for each independent line (C1 and C2).

FIG. 5: Distribution of plants in function of their fresh weight. The graphs represent the frequency of a plant belonging to a certain category of weight.

FIG. 6: Distribution of plants in function of their fresh weight. The graphs represent the frequency of a plant belonging to a certain category of weight (% of plants versus fresh weight category, as in FIG. 5). The lines analyzed are independent lines of the ones presented in FIG. 5.

### DETAILED DESCRIPTION OF THE INVENTION

### EXAMPLES

### MATERIALS AND METHODS TO THE EXAMPLES

### Cloning and TAP Purification

Cloning of transgenes encoding tag fusions under control of the constitutive Cauliflower tobacco mosaic virus 35S promoter, transformation of Arabidopsis cell suspension cultures, protein extract preparation, TAP purification, protein precipitation and separation were done as previously described (Van Leene et al., 2007). The adapted protocol used for purification of protein complexes incorporating GS-tagged bait is described elsewhere (Van Leen et al., 2008). For identification by mass spectrometry, minor adjustments were implemented compared to previously described protocols (Van Leene et al., 2007), as described below.

### Proteolysis and Peptide Isolation

After destaining, gel slabs were washed for 1 hour in H₂O, polypeptide disulfide bridges were reduced for 40 minutes in 25 mL of 6.66 mM DTT in 50 mM NH₄HCO₃ and, sequentially, the thiol groups were alkylated for 30 minutes in 25 mL 55 mM IAM in 50 mM NH₄HCO₃. After washing the gel slabs three times with water, complete lanes from the protein gels were cut into slices, collected in microtiter plates and treated essentially as described before with minor modifications (Van Leene et al., 2007). Per microtiter plate well, dehydrated gel particles were rehydrated in 20 μL digest buffer containing 250 ng trypsin (MS Gold; Promega, Madison, Wis.), 50 mM NH₄HCO₃ and 10% CH₃CN (v/v) for 30 minutes at 4° C. After adding 10 μL of a buffer containing 50 mM NH₄HCO₃ and 10% CH₃CN (v/v), proteins were digested at 37° C. for 3 hours. The resulting peptides were concentrated and desalted with microcolumn solid phase tips (PerfectPure™ C18 tip, 200 nL bed volume; Eppendorf, Hamburg, Germany) and eluted directly onto a MALDI target plate (Opti-TOFTM384 Well Insert; Applied Biosystems, Foster City, Calif.) using 1.2 μL of 50% CH₃CN:0.1% CF₃COOH solution saturated with α-cyano-4-hydroxycinnamic acid and spiked with 20 fmole/μL Glu1 Fibrinopeptide B (Sigma Aldrich), 20 fmole/μL des-Pro2-Bradykinin (Sigma Aldrich), and 20 fmole/μL Adrenocorticotropic Hormone Fragment 18-39 human (Sigma Aldrich).

### Acquisition of Mass Spectra

A MALDI tandem MS instrument (4700 and 4800 Proteomics Analyzer; Applied Biosystems) was used to acquire peptide mass fingerprints and subsequent 1 kV CID fragmentation spectra of selected peptides. Peptide mass spectra and peptide sequence spectra were obtained using the settings essentially as previously presented (Van Leene et al., 2007). Each MALDI plate was calibrated according to the manufacturers' specifications. All peptide mass fingerprinting (PMF) spectra were internally calibrated with three internal standards at m/z 963.516 (des-Pro2-Bradykinin), m/z 1570.677 (Glu1-Fibrinopeptide B), and m/z 2465,198 (Adrenocorticotropic Hormone Fragment 18-39) resulting in an average mass accuracy of 5 ppm±10 ppm for each analyzed peptide spot on the analyzed MALDI targets. Using the individual PMF spectra, up to sixteen peptides, exceeding a signal-to-noise ratio of 20 that passed through a mass exclusion filter, were submitted to fragmentation analysis.

### MS-Based Protein Homology Identification

PMF spectra and the peptide sequence spectra of each sample were processed using the accompanied software suite (GPS Explorer 3.6, Applied Biosystems) with parameter settings essentially as previously described (Van Leene et al, 2007). Data search files were generated and submitted for protein homology identification by using a local database search engine (Mascot 2.1, Matrix Science). An in-house non-redundant Arabidopsis protein database called SNAPS Arabidopsis thaliana version 0.4 (SNAPS=Simple Non-redundant Assembly of Protein Sequences, 77488 sequence entries, 30468560 residues; available at WorldWideWeb.ptools.ua.ac.be/snaps) was compiled from nine public databases. Protein homology identifications of the top hit (first rank) with a relative score exceeding 95% probability were retained. Additional positive identifications (second rank and more) were retained when the score exceeded the 98% probability threshold. Because identifications were done with different versions of the SNAPS database (Van Leen et al., 2007), and with the goal to obtain more uniformity between the identifications, all identifications from the core and the non-core dataset were resubmitted to Mascot and identified with the protein sequence repertoire from the latest TAIR database (TAIR8.0). Furthermore, an additional restriction was implemented to reduce the number of false positive identifications and, as so, identifications for which more than 50% of the corresponding peptides had a trypsin miss-cleavage, were discarded.

### GO Enrichment Analysis

Analysis of over- and under-representation of GO terms was done with the BiNGO tool (Maere et al., 2005) in Cytoscape (Shannon et al., 2003). The hypergeometric test was chosen at a significance value of 0.05 with the Benjamini and Hochberg False Discovery Rate Correction for multiple testing. The Arabidopsis gene annotation file used in the analysis was downloaded from the gene ontology website on the 4th of October 2008.

### Cell Cycle Enrichment Analysis

For the periodic gene enrichment analysis, a list of 1258 genes showing cell cycle-regulated and cell cycle-associated expression was compiled from two datasets (Menges et al., 2003; Jensen et al., 2006). Genome wide corresponds to all 23834 genes present on the Affymtetrix ATH1 microarray. Genes containing E2F or M-specific activator (MSA) motifs in their promoter sequence were in silico determined by combining transcript expression data and comparative genomics (Vandepoele et al, 2006). Here, genome wide corresponds to 19173 genes for which a unique probe set is available on the ATH1 microarray. Proteins containing the CDK consensus phosphorylation site [ST]PX[KR], a known hallmark of CDK substrates (De Veylder et al., 1997), were considered as potential CDK substrates. The presence of the consensus motif was screened with the patmatch tool available at TAIR and, hence, genome wide corresponds here to all 27235 proteins present in the TAIR8.0 release. For all enrichment analysis, p-values were calculated with the hypergeometric cumulative distribution function of the Matlab 7.5 software. Proteins that could not be assigned to a specific gene locus were discarded from all enrichment analysis.

Overlap with Protein-Protein Databases

To assess the novelty of the cell cycle interactome, we screened for the overlap of our datasets with the following databases containing protein-protein interactions: TAIR (Huala et al., 2001), InTact (Kerrien et al., 2007), Arabidopsis Reactome (Tsesmetzis et al., 2008), AtPID (Cui et al., 2008), Reactome (Vastrik et al., 2007) and The Bio-Array Resource (BAR) for Arabidopsis Functional Genomics (Geisler-Lee et al., 2007).

### Co-Expression Analysis

Transcript Pearson Correlation Coefficients (PCC), representing the degree of co-expression of gene pairs, were calculated based on an Arabidopsis ATH1 micro-array compendium of 518 experiments focused towards cell cycle or plant growth and development (Table 7). We compared the PCC distribution of both datasets with the PCC distribution of 100 randomized datasets with an equal number of randomly chosen proteins and interactions.

### Constructs and Plant Transformation

The overexpressing constructs were produced by using gateway cloning technology. The cDNA of the genes of interest (table, sheet OE produced and LOF requested) were amplified by PCR from reverse transcribed RNA extracted from tissues of Arabidopsis thaliana ecotype Columbia. The PCR reactions were performed using the Phusion High fidelity DNA polymerase (Finnzymes) according to the manufacturer's instructions. The PCR fragments, corresponding to complete cDNA of the genes of interest were introduced into pDONr 201 using the Gateway system (Invitrogen) by attBXattP recombination sites and subsequently recombined into the pK7WG2 expression vector by attL XattR sites recombination. The sequence was confirmed by sequencing. The constructs containing the genes of interest under the control of the CaMV 35S promoter were used to transform Arabidopsis thaliana by the flowerdip method (Clough and Bent, 1998).

### Plant Material and Growth Condition

Transgenic lines were identified by selection on MS medium (half-strength Murashige and Skoog medium (Duchefa, Haarlem, The Netherlands), Sucrose 1%) supplemented with 50 mg/l kanamycin and later transferred to soil for seed production. A second selection on MS plus kanamycine allowed the selection of lines containing one site of insertion of the transgene. Plants were grown under a 16-hour day and 8-hour night regime at 21° C.

### Biomass Test

For the biomass measurement, the vegetative part of a 20-day-old plant grown on MS medium was harvested and the fresh weight was measured by weighing about 60 plants of each line.

### Example 1

### Isolation of the TAP Complexes

As baits, we used 73 “core” cell cycle regulatory proteins (Vandepoele et al., 2002; Menges et al., 2006; Perez et al., 2007), four mitotic checkpoint proteins (Menges et al., 2005), eight anaphase promoting complex (APC) subunits and six APC activators (Capron et al., 2003), one 26S proteasome subunit (Brukhin et al., 2005), ten proteins involved in DNA replication or repair (Schultz et al., 2007), and as proof of concept, six proteins for reverse TAP experiments (Table 1). Of the 108 TAP fusions, 102 were expressed successfully. In total, 303 purifications were performed with at least two independent purifications per bait.

### Example 2

### Identification of the Prey Proteins

Purified proteins were identified via MALDI-TOFTOF. Non-specific proteins, determined by control purifications, were subtracted from the hit lists (Table 2), generating a non-redundant dataset of 857 interactions among 393 proteins. This dataset was divided in a “core” dataset of 371 interactions among 196 proteins, containing interactions that were biologically confirmed in at least two independent repeats or in the reciprocal experiment, and a “non-core” dataset with the remainder 486 interactions among 320 proteins.

### Example 3

### Enrichment Analysis

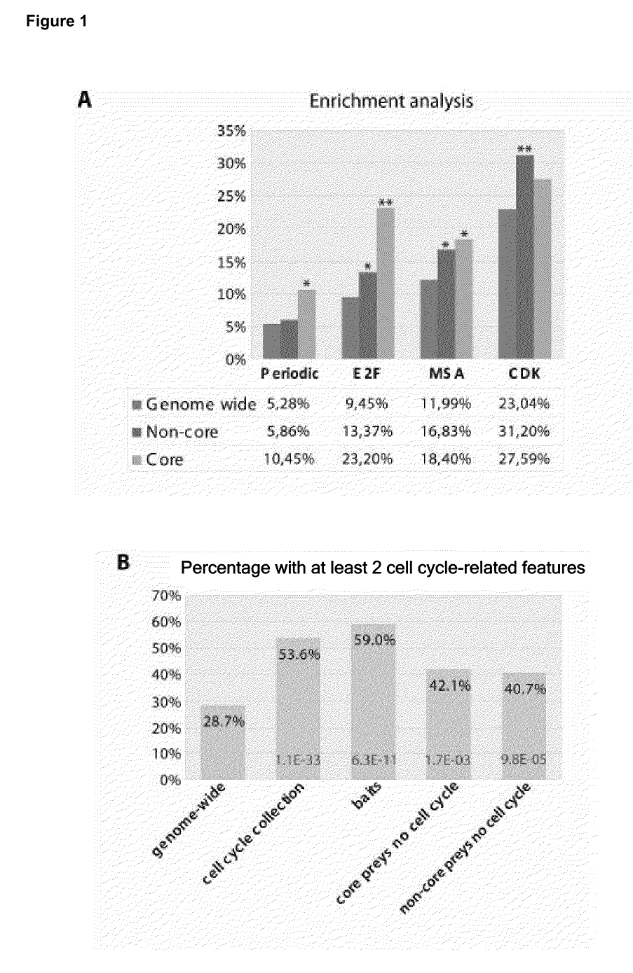

To assess the quality of the interactome, we performed different enrichment analysis on the core and non-core preys. In both datasets, the GO term “cell cycle” was highly enriched (Table 3). Additional GO enrichments demonstrate that cell cycle is linked to a myriad of biological processes including growth and development, response to stress and hormone stimuli, energy production, chromatin remodeling and others. Next, we observed an enrichment in the core dataset for genes periodically expressed during cell cycle (FIG. 1, Panel A). Furthermore, both datasets were enriched for genes with E2F or M-specific activator (MSA) motifs in their promoter. The less pronounced enrichments for the non-core dataset indicate that it is more biased for interactions linking the core cell cycle machinery with other pathways. This is supported by the fact that the non-core dataset is more enriched for potential CDK substrates, as assessed by the presence of CDK phosphorylation sites. The fact that these interactions were often not confirmed is likely due to the transient nature of, e.g., CDK/substrate interactions.

### Example 4

### Isolation of Novel Cell Cycle Genes

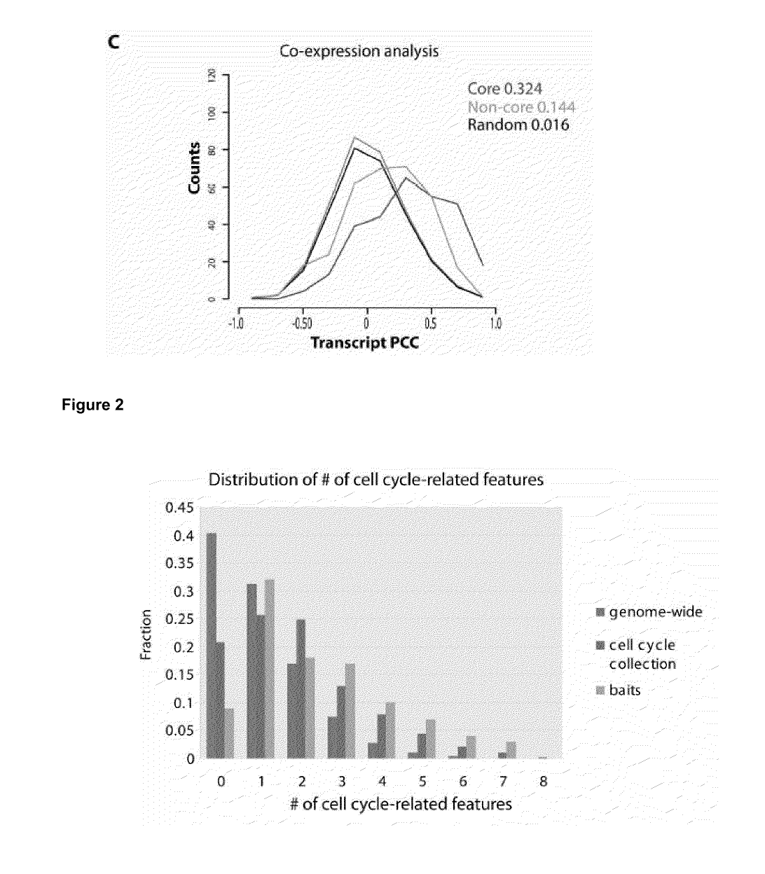

In a quest for new cell cycle-related proteins, we integrated different cell cycle-related features (Table 4). The distribution of the number of features per gene shows that a collection of known cell cycle genes (Table 5) is enriched for these features compared to the whole gene pool (FIG. 2). A clear shift between the whole gene pool and the cell cycle collection was visible at two features, as it was the case for the original bait list, validating the choice of our baits. In search for new cell cycle proteins, we started from the core and non-core prey list and subtracted the collection of known cell cycle genes. The percentage meeting the criterion of having at least two features is shown (FIG. 1, Panel B), and all classes are significantly enriched compared to the whole gene pool. By filtering for genes containing at least two features, we generated a list of 123 potential new cell cycle genes from the core and non-core dataset (Table 6).

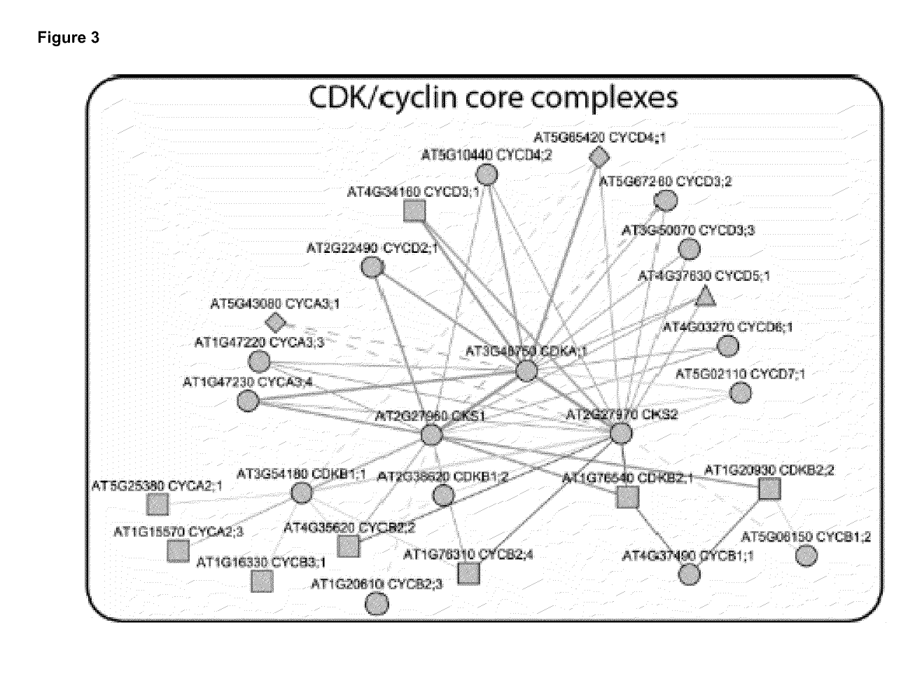

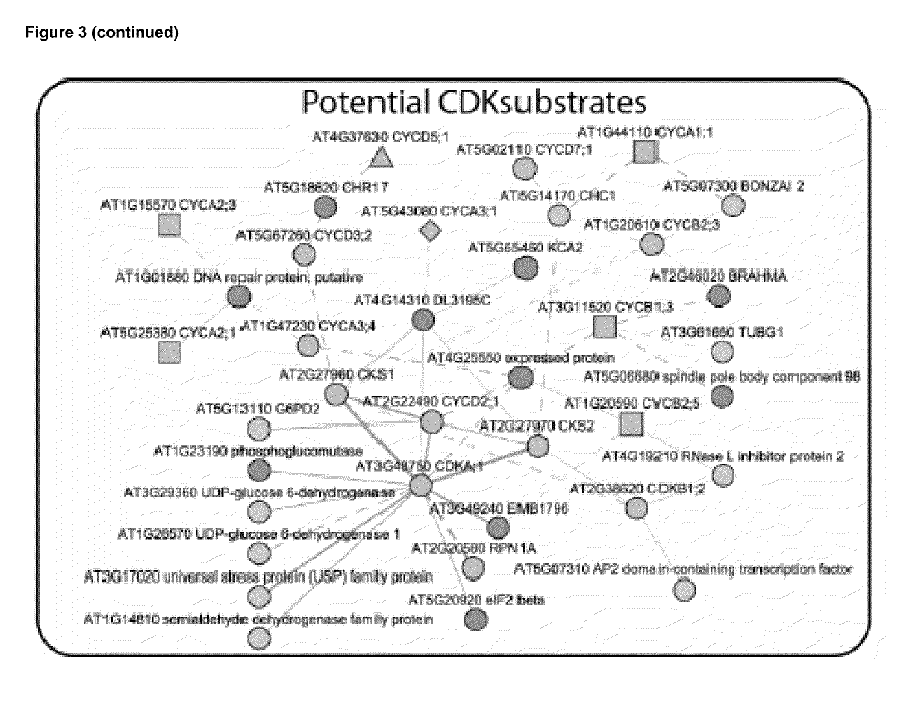

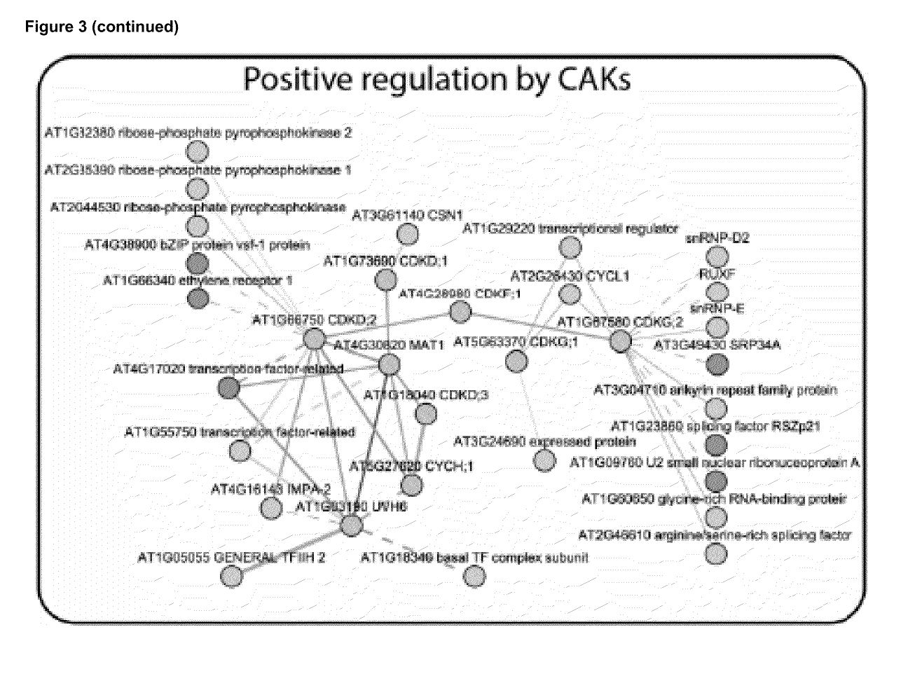

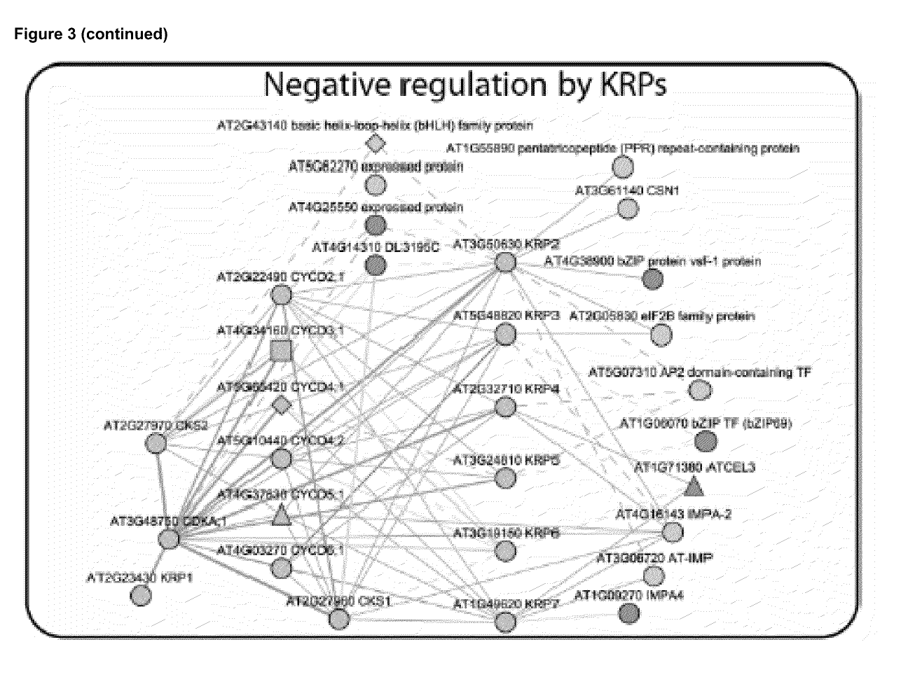

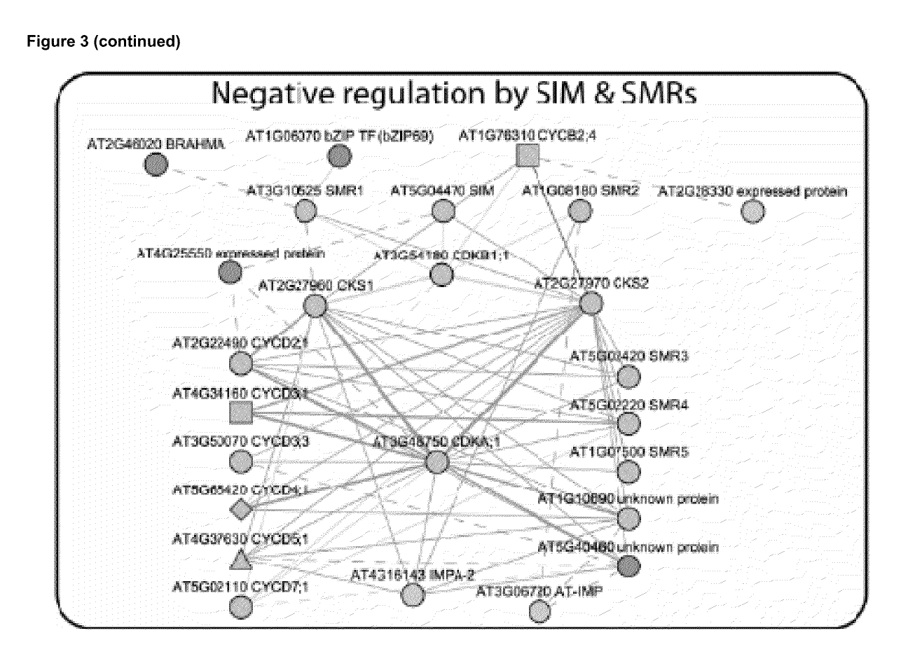

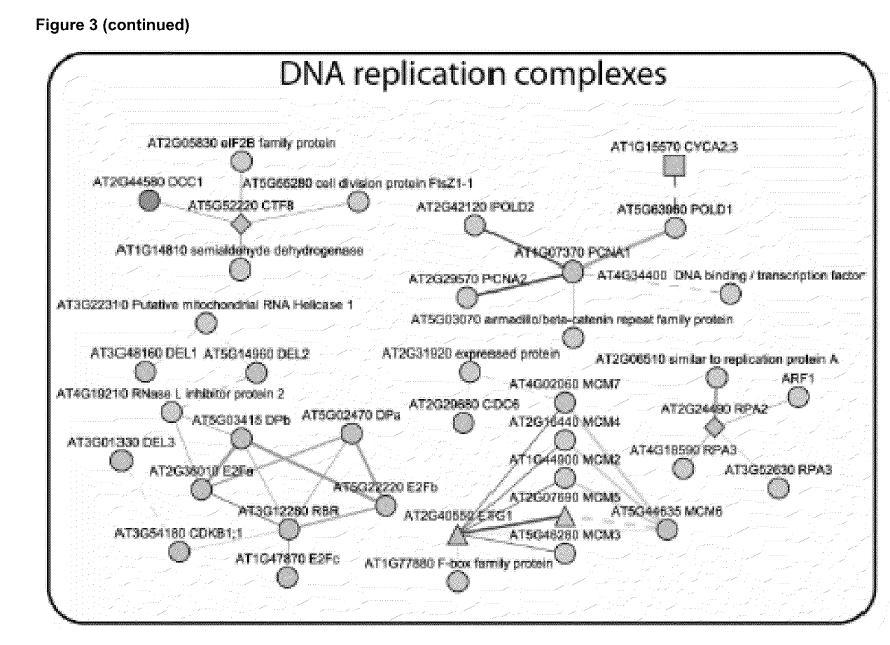

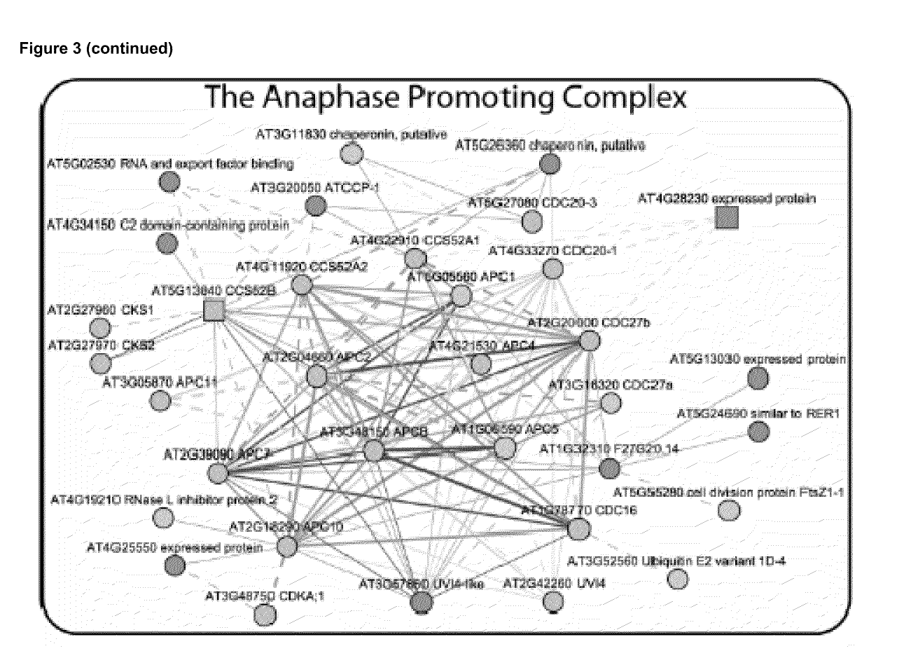

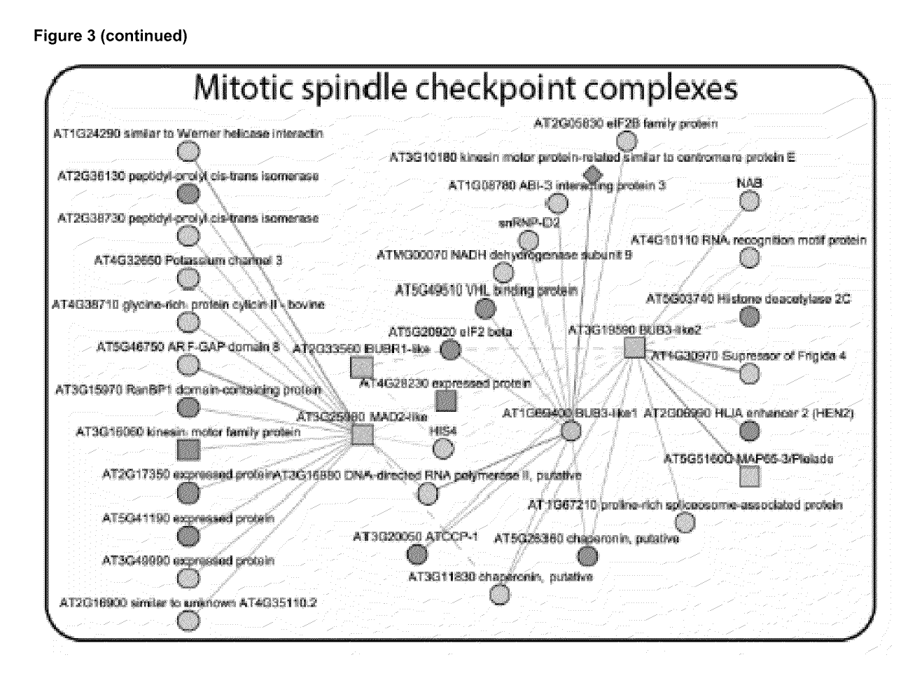

The robustness of the data is further exemplified by the observation that 46% of the core and 8% of the non-core dataset interactions are between baits, as our baits are supposed to act in common pathways. Screening our data for overlap with existing protein-protein interaction databases learned that 66% of the core and 95% of the non-core dataset interactions are new. On the other hand, this implicates that one-third of the core dataset is validated by other means. Finally, interactions from both datasets tend to be more co-expressed compared to interactions from randomized datasets as assessed by calculation of the transcript Pearson Correlation Coefficient (PCC) (FIG. 1, Panel C). The integrated cell cycle interactome can be accessed through a Cytoscape webstart and is provided as a matrix pivot table that allows easy querying of the interactome. To demonstrate the biological importance of the interactome, a selection of subnetworks is discussed below, covering interactions from both the core and non-core dataset (FIG. 3).

### Example 5

### Isolated Genes of the CDK Complexes

Key players in cell cycle progression are cyclin-dependent kinase (CDK) complexes. CDKA;1, the Arabidopsis ortholog of yeast cdc2/cdc28, co-purified with all tested D-type cyclins and with A3-type cyclins, but not with the mitotic A1-, A2- or B-type cyclins. Combining our interactome data with expression data (Menges et al. 2005), we speculate that at cell cycle reentry and early in G1-phase, CDKA;1 binds CYCD3;3 and CYCD5;1. Further on in G1-phase and at the G1/S checkpoint, CDKA;1 binds a variety of D-type cyclins, such as CYCD4;1, CYCD4;2, CYCD3;1, CYCD6;1 and CYCD7;1. In addition, CDKA;1 interacts with S-phase specific A3-type cyclins. The other A- and B-type cyclins, of which most possess a peak of expression at the G2/M-boundary, bind the plant-specific mitotic B-type CDKs. B1-type cyclins associate exclusively with B2-type CDKs, while the remainder A- and B-type cyclins preferentially bind B1-type CDKs. Although transient interactions are more difficult to screen with TAP, our interactome contains different potential CDK substrates. As predicted (Geisler-Lee et al., 2007), CDKA;1 is present as a highly connected hub in the core network. It co-purified the unknown protein AT4G14310, which was further present in complexes with CKS1, CKS2, CYCA3;1, CyCA3;4 and KRP2 and the reverse purification confirmed interaction with CDKA;1 and CKS2 and revealed interaction with the plant-specific kinesin motor protein KCA2 involved in division plane determination. Next, CDKA;1 was pulled down with the 26S proteasome complex, purified through RPN1a, possibly reflecting cell cycle regulation of the 26S proteasome. CDKA;1 further interacted with three proteins from the UDP-xylose biosynthesis pathway, coupling cell cycle regulation with cell wall synthesis (Siefert, 2004). With three A-type cyclins, we picked up a DNA repair protein and with CYCB1;3, we found γ-tubulin and a spindle pole body component, two proteins involved in microtubule nucleation during, e.g., assembly of the preprophase band, a plant-specific structure required for polarity determination during cell cycle (Erhardt et al., 2002). Furthermore, some interesting chromatin-remodeling proteins were identified with different cyclins: CHR17, an E2F-up-regulated ISWI protein (Huanca-Mamani et al., 2005) interacted with CYCD3;2 and CYCD5;1. CHC1 associated with CYCA1;1, CYCD7;1, CYCB2;3 and CKS2, and BRAHMA, a SWI/SNF chromatin-remodeling ATPase implicated in the formation and/or maintenance of cotyledon boundary cells during embryogenesis (Kwon et al., 2006), was identified with CYCB1;3 and CYCB2;3.

### Example 6

### Isolation of Positive Regulators of the Cell Cycle

For full activity, CDKs require, next to cyclin binding, phosphorylation of a threonine residue within the T-loop by CDK-activating kinases (CAK). The Arabidopsis genome encodes four CAKs, namely, three D-type CDKs, homologous to human CDK7, and one cyclin-independent CAK-activating kinase (CAKAK) CDKF;1. Here, we show that both CDKD;2 and CDKD;3 form a trimeric complex with CYCH;1 and the CAK assembly factor MAT1. Like in rice (Rohila et al., 2006), CDKD;2 is also part of the basal TFIIH complex involved in transcription and DNA repair, as three members co-purified (UVH6/XPD, AT1G55750 and AT4G17020). In this complex, CDKD;2 activates transcription through phosphorylation of the C-Terminal Domain (CTD) of RNA polymerase II. With UVH6 and MAT1 as baits, we confirmed interaction with CDKD;2 and purified two more proteins of the TFIIH complex. CDKD;2 further co-purified proteins involved in nucleotide biosynthesis, namely, three ribose-phosphate pyrophosphokinases. More upstream, the monomeric CAKAK CDKF;1 activates CDKD;2 in a cyclin-independent manner. On the other hand, CDKF;1 also binds CDKG;2. The G-type CDK class has two members in Arabidopsis, and is homologous to the human cytokinesis-associated p58 galactosyltransferase protein. Here, we discovered CYCL1, a cyclin with a SR-like splicing domain (Forment et al., 2002), as the regulatory cyclin partner of both G-type CDKs, validating the clustering of CYCL1 with CDKG;2 in a tissue-specific gene expression analysis (Menges et al., 2005). Both core and non-core interacting proteins hint for a function of CDKG/CYCL complexes in regulation of transcription and splicing, so activation of CDKF;1 could lead to altered splicing events during cell proliferation.

### Example 7

### Isolation of Negative Regulators of the Cell Cycle

Negative regulation of cell cycle progression is achieved by docking of small proteins to the CDK/cyclin complexes. Arabidopsis encodes seven proteins related to the mammalian Kip/Cip inhibitors, known as Kip-related proteins (KRPs). Here, we show that all KRPs, except KRP1, interact with both CDKA;1 and D-type cyclins. With three KRPs, CDKB1;2 and two APC activators, we found an ethylene responsive AP2 transcription factor (TF), and with KRP2 we picked up a bZIP TF also found with B-type cyclins and CDKB1;2. In plants, a second family of cell cycle inhibitor proteins exist that are up-regulated by abiotic and biotic stress, comprising SIAMESE (SIM) and SIAMESE-Related (SMR) proteins (Peres et al., 2007; Churchman et al., 2006). SIM is a nuclear protein promoting endoreduplication in trichomes by suppression of mitosis. It was proposed that it inhibits mitosis through inhibition of CDKA;1/CYCD complexes (Churchman et al., 2006). In our dataset, however, SIM co-purifies CDKB1;1 and not CDKA;1, so endoreduplication may be triggered directly by inhibiting mitotic CDKB/cyclin complexes. Next to SIM, also SMR1 and SMR2 associate with CDKB1;1, and the CDKB1;1 interactor CYCB2;4 binds AT2G28330, an additional member of the SMR family. In contrast, SMR3-5 bind CDKA;1 and D-type cyclins. Besides, with CDKA;1 and different D-type cyclins as bait, we picked up two new members of the SMR clan, AT5G40460 and AT1G10690, and reverse purifications confirmed these interactions. As AT5G40460 was almost 20-fold induced in plants overexpressing E2Fa and DPa (Vandepoele et al., 2005), it may inhibit CDKA;1/CYCD complexes during S-phase preventing re-initiation of DNA replication. SMR1 further co-purified bZIP69, a TF also found with KRP3 and KRP5. Importins often co-purified both with KRPs and SMRs, supporting the importance of the regulation of their subcellular localization for their activity.

### Example 8

### Isolation of Genes Involved in Nucleotide Synthesis, DNA Replication and DNA Repair

At the G1/S boundary, CDKs activate the E2F/DP pathway by phosphorylation of the repressor RBR, inducing transcription of genes mainly involved in nucleotide synthesis, DNA replication and DNA repair. We demonstrate that E2Fa and E2Fb can associate both with DPa and DPb, and that all E2F and DP proteins co-purify RBR. Since CDKB1;1 interacted with DEL3, an atypical E2F protein lacking the trans-activation domain, we propose that DEL3 is regulated by CDKB1;1 activity, consistent with a second expression peak of DEL3 at G2/M (Menges et al., 2005). Interestingly, the mitotic CDKB1;1, and not CDKA;1, co-purified with RBR, providing further evidence that the E2F/DP/RBR network is not only active at G1/S but also at G2/M transitions, as was previously suggested in plants (Magyar et al., 2005) and mammalian cells (Ishida et al., 2001), or that mitotic CDK/cyclin complexes are active during S-phase as in yeast (Wuarin et al., 2002). We further identified some complexes involved in DNA replication, like the MCM complex, possessing helicase activity for unwinding of double-stranded DNA during DNA replication. This complex was isolated with MCM6 as bait, together with the recently published (Takahashi et al., 2008) and highly co-expressed E2F-target gene 1 (ETG1). The co-purified fraction of proliferating cell nuclear antigen 1 (PCNA1), a sliding clamp for DNA polymerase and thus a key actor in DNA replication, contained PCNA2, two DNA polymerase delta subunits (POLD1-2), of which one also interacted with CYCA2;3, an armadillo/beta-catenin repeat family of unknown function and a DNA binding protein. Furthermore, we prove the existence of the alternative Ctf18 replication factor C complex in plants, required for sister chromatid cohesion in yeast (Mayer et al., 2001) and a protein complex involved in stabilization of single-stranded DNA during replication, repair and transcription, including RPA2, two RPA3 proteins and a putative replication protein (Schultz et al., 2007).

### Example 9

### Analysis of Overexpression Lines

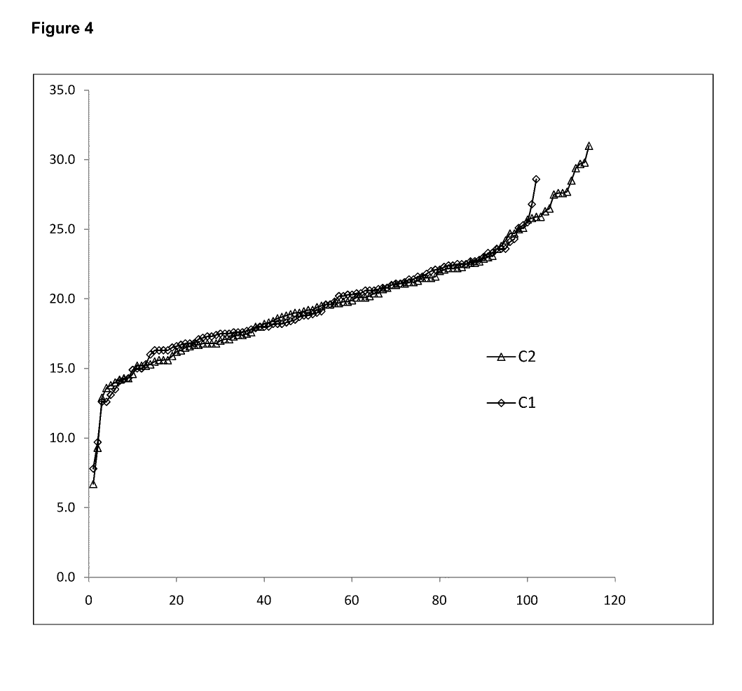

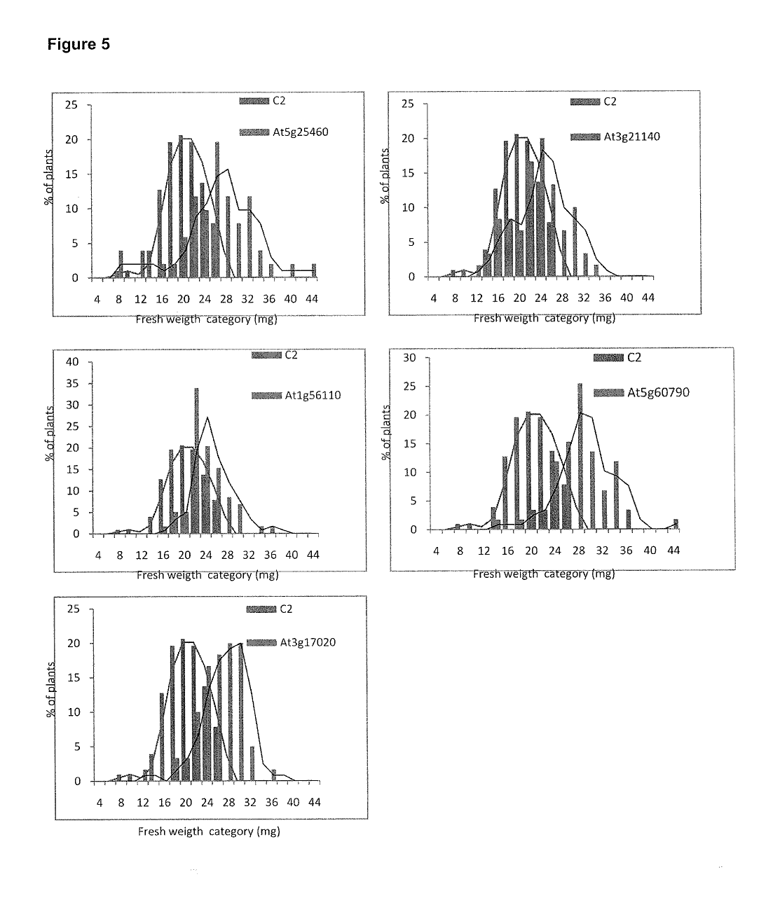

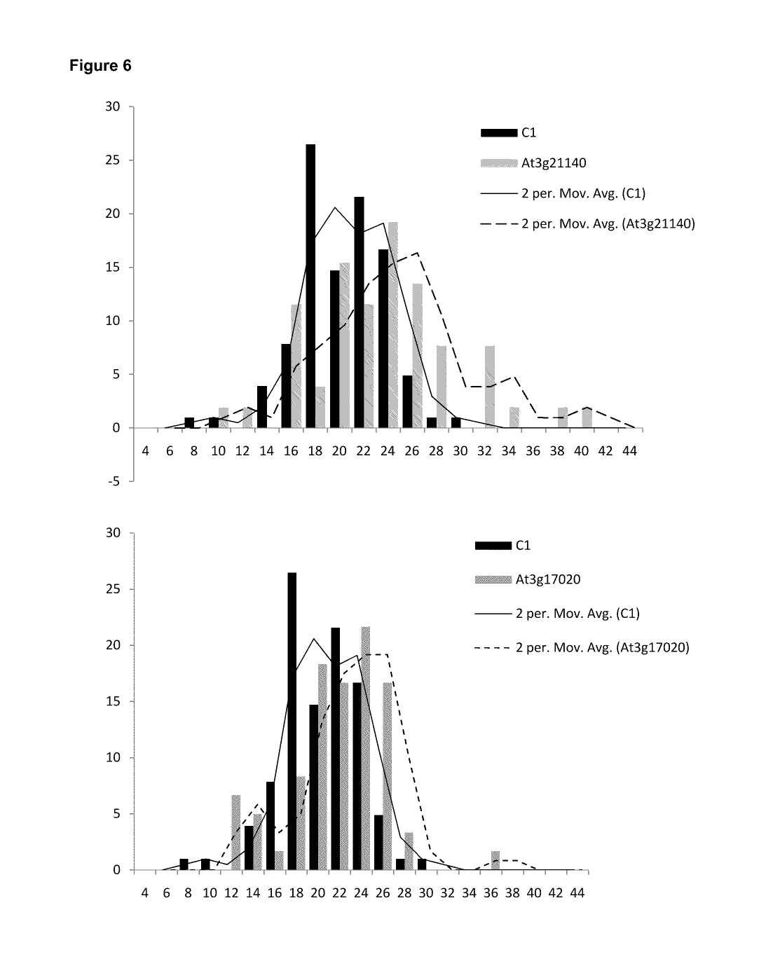

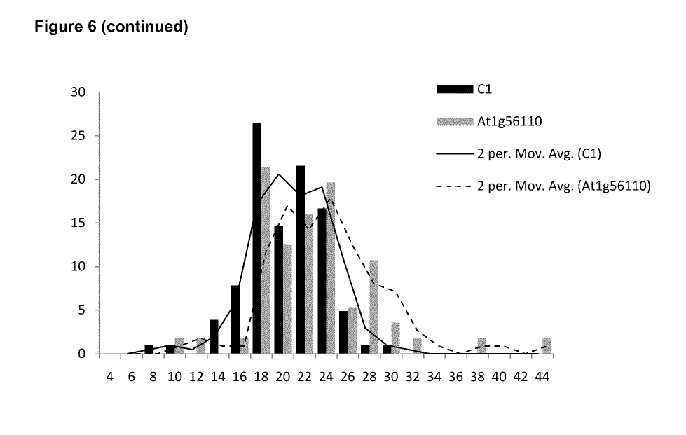

Thirty-two genes were cloned into expression vectors using Gateway cloning to produce plants overexpressing the genes of interest under the control of the CaMV35S promoter (table, sheet OE produced and LOF requested). These constructs were used for Arabidopsis thaliana transformation using the flower dip method. Primo-transformants were selected for all the constructs and grown for seed production. The seeds from eleven constructs (At5g25460, At3g01280, At1g31760, At3g21140, At3g17020, At1g05805, At1g10690, At1g56110, At5g24690, At5g60790, At1g09760) were harvested and used to select lines having one site of insertion of the transgene. A biomass test has been carried out on these segregating populations containing one insertion site. In this population, it is expected to find ¼ of wt, 2/4 of hemizygous and ¼ of homozygous plants. When the overexpression of a transgene leads to the production of larger plants, a fraction (¼ or ¾) of the plants analyzed will show an increased biomass. This method allows a fast screening of genes of which the overexpression gives a positive effect on plant growth. Plants were then grown under in vitro condition and the rosette fresh weight of approximately 60 plants was measured 20 days after stratification (table, sheet OE data+average). The control plants used in this experiment correspond to segregating plants transformed with an empty vector (C1 and C2). As shown in FIG. 4, for these two independent control lines, the plant fresh weight varies from 8 mg to 28 mg with an average of 20 mg. Among the eleven overexpressing lines analyzed, we found that for five (At1g56110, At3g17020, At3g21140, At5g25460, At5g60790), this distribution of weight is shifted towards larger plants than in the controls (FIG. 5). This shift in biomass range was found for three of these lines in two independent transformants (FIG. 6). The presence of larger plants in this segregating population proves that these five genes are involved in the control of growth.

TABLE 1
Overview of bait proteins used to elucidate the core cell cycle interactome of
Arabidopsis thaliana.
Topology = C- or N-terminal tag fusions. Tag refers to the applied tag
being either the traditional TAP tag developed for Saccharomyces cerevisiae (15),
or an optimized dual affinity tag (GS) (2). Expression was indicated as (+) if the
TAP fusion protein could be detected by western blot analysis. The total number of
purifications performed per bait is shown, with a minimum of two experiments.

| Bait | Locus | Category | Topology | Tag | Expressed | Purifications |
|---|---|---|---|---|---|---|
| CDKA; 1 | AT3G48750 | core cell cycle | C + N | TAP | + | 11 |
| CDKB1; 1 | AT3G54180 | core cell cycle | C + N | TAP | + | 4 |
| CDKB1; 2 | AT2G38620 | core cell cycle | C | TAP | + | 2 |
| CDKB2; 1 | AT1G76540 | core cell cycle | N | TAP | + | 2 |
| CDKB2; 2 | AT1G20930 | core cell cycle | C | TAP | + | 4 |
| CDKC; 1 | AT5G10270 | core cell cycle | C | TAP | + | 2 |
| CDKC; 2 | AT5G64960 | core cell cycle | C | TAP | + | 4 |
| CDKD; 1 | AT1G73690 | core cell cycle | C | TAP | + | 2 |
| CDKD; 2 | AT1G66750 | core cell cycle | C + N | TAP + GS | + | 10 |
| CDKD; 3 | AT1G18040 | core cell cycle | C | TAP | + | 2 |
| CDKE; 1 | AT5G63610 | core cell cycle | N | TAP | + | 2 |
| CDKF; 1 | AT4G28980 | core cell cycle | C | TAP | + | 3 |
| CDKG; 1 | AT5G63370 | core cell cycle | C | GS | + | 2 |
| CDKG; 2 | AT1G67580 | core cell cycle | C | TAP | + | 2 |
| CKS1 | AT2G27960 | core cell cycle | C | TAP + GS | + | 10 |
| CKS2 | AT2G27970 | core cell cycle | C | TAP | + | 2 |
| CYCA1; 1 | AT1G44110 | core cell cycle | C | TAP | + | 2 |
| CYCA1; 2 | AT1G77390 | core cell cycle | C | TAP | − | |
| CYCA2; 1 | AT5G25380 | core cell cycle | N | TAP | + | 2 |
| CYCA2; 2 | AT5G11300 | core cell cycle | C | TAP | + | 4 |
| CYCA2; 3 | AT1G15570 | core cell cycle | N | TAP | + | 4 |
| CYCA2; 4 | AT1G80370 | core cell cycle | C | TAP | − | |
| CYCA3; 1 | AT5G43080 | core cell cycle | C | TAP | + | 4 |
| CYCA3; 2 | AT1G47210 | core cell cycle | N | TAP | − | |
| CYCA3; 3 | AT1G47220 | core cell cycle | N | GS | + | 2 |
| CYCA3; 4 | AT1G47230 | core cell cycle | C | TAP | + | 4 |
| CYCB1; 1 | AT4G37490 | core cell cycle | C | TAP + GS | + | 4 |
| CYCB1; 2 | AT5G06150 | core cell cycle | C | TAP | + | 4 |
| CYCB1; 3 | AT3G11520 | core cell cycle | C | TAP | + | 2 |
| CYCB1; 4 | AT2G26760 | core cell cycle | C | TAP | + | 2 |
| CYCB2; 1 | AT2G17620 | core cell cycle | N | TAP | + | 2 |
| CYCB2; 2 | AT4G35620 | core cell cycle | C | TAP | + | 5 |
| CYCB2; 3 | AT1G20610 | core cell cycle | C | TAP | + | 4 |
| CYCB2; 4 | AT1G76310 | core cell cycle | C | GS | + | 2 |
| CYCB2; 5 | AT1G20590 | core cell cycle | C | TAP | + | 2 |
| CYCB3; 1 | AT1G16330 | core cell cycle | N | TAP | + | 4 |
| CYCD1; 1 | AT1G70210 | core cell cycle | C | TAP | − | |
| CYCD2; 1 | AT2G22490 | core cell cycle | C | TAP | + | 6 |
| CYCD3; 1 | AT4G34160 | core cell cycle | N | TAP | + | 2 |
| CYCD3; 2 | AT5G67260 | core cell cycle | C | TAP | + | 3 |
| CYCD3; 3 | AT3G50070 | core cell cycle | C | TAP | + | 4 |
| CYCD4; 1 | AT5G65420 | core cell cycle | C | TAP | + | 2 |
| CYCD4; 2 | AT5G10440 | core cell cycle | C | TAP | + | 2 |
| CYCD5; 1 | AT4G37630 | core cell cycle | C | TAP | + | 2 |
| CYCD6; 1 | AT4G03270 | core cell cycle | C | TAP | + | 2 |
| CYCD7; 1 | AT5G02110 | core cell cycle | C | GS | + | 2 |
| CYCH; 1 | AT5G27620 | core cell cycle | N | TAP | + | 4 |
| CYCT1; 3 | AT1G27630 | core cell cycle | C | TAP | + | 2 |
| DEL1 | AT3G48160 | core cell cycle | N | TAP | + | 2 |
| DEL2 | AT5G14960 | core cell cycle | N | TAP | + | 2 |
| DEL3 | AT3G01330 | core cell cycle | N | TAP | + | 2 |
| DPa | AT5G02470 | core cell cycle | C | TAP | + | 2 |
| DPb | AT5G03415 | core cell cycle | N | TAP | + | 2 |
| E2Fa | AT2G36010 | core cell cycle | C | TAP + GS | + | 5 |
| E2Fb | AT5G22220 | core cell cycle | N | TAP | + | 2 |
| E2Fc | AT1G47870 | core cell cycle | C | TAP | + | 5 |
| KRP1 | AT2G23430 | core cell cycle | N | TAP | + | 2 |
| KRP2 | AT3G50630 | core cell cycle | N | TAP | + | 6 |
| KRP3 | AT5G48820 | core cell cycle | N | TAP | + | 4 |
| KRP4 | AT2G32710 | core cell cycle | N | TAP | + | 6 |
| KRP5 | AT3G24810 | core cell cycle | N | TAP | + | 2 |
| KRP6 | AT3G19150 | core cell cycle | N | TAP | + | 4 |
| KRP7 | AT1G49620 | core cell cycle | N | TAP | + | 2 |
| RBR | AT3G12280 | core cell cycle | N | TAP | + | 2 |
| WEE1 | AT1G02970 | core cell cycle | N | TAP | + | 4 |
| Cdc25-like | AT5G03455 | core cell cycle | C | TAP | + | 2 |
| MAT1 | AT4G30820 | core cell cycle | N | TAP | + | 2 |
| SIM | AT5G04470 | core cell cycle | C | TAP | + | 4 |
| SMR1 | AT3G10525 | core cell cycle | C | TAP | + | 4 |
| SMR2 | AT1G08180 | core cell cycle | C | TAP | + | 2 |
| SMR3 | AT5G02420 | core cell cycle | C | TAP | + | 2 |
| SMR4 | AT5G02220 | core cell cycle | C + N | TAP | + | 4 |
| SMR5 | AT1G07500 | core cell cycle | N | GS | + | 2 |
| APC2 | AT2G04660 | APC core | N | TAP | + | 2 |
| APC4 | AT4G21530 | APC core | C | TAP | − | |
| APC7 | AT2G39090 | APC core | N | TAP | + | 2 |
| APC8 | AT3G48150 | APC core | N | TAP | + | 2 |
| APC10 | AT2G18290 | APC core | N | TAP | + | 4 |
| APC11 | AT3G05870 | APC core | C | TAP | + | 2 |
| CDC16 | AT1G78770 | APC core | C | TAP | + | 2 |
| CDC27B | AT2G20000 | APC core | C | TAP | + | 2 |
| CCS52A1 | AT4G22910 | APC activator | N | TAP | + | 4 |
| CCS52A2 | AT4G11920 | APC activator | N | TAP | + | 6 |
| CCS52B | AT5G13840 | APC activator | N | TAP | + | 4 |
| CDC20.1 | AT4G33270 | APC activator | N | TAP | + | 2 |
| CDC20.3 | AT5G27080 | APC activator | N | TAP | + | 2 |
| CDC20.6 | AT5G27945 | APC activator | C | TAP | − | |
| RPN1a | AT2G20580 | 26S proteasome | N | TAP | + | 2 |
| CDC6 | AT2G29680 | DNA replication | N | TAP | + | 2 |
| CDC6b | AT1G07270 | DNA replication | C | TAP | + | 2 |
| CTF8 | AT5G52220 | DNA replication | N | GS | + | 2 |
| ETG1 | AT2G40550 | DNA replication | C + N | TAP | + | 4 |
| MCM6 | AT5G44635 | DNA replication | C | TAP | + | 2 |
| MCM7 | AT4G02060 | DNA replication | C | TAP | + | 2 |
| ORC1a | AT4G14700 | DNA replication | N | TAP | + | 2 |
| PCNA1 | AT1G07370 | DNA replication | N | TAP | + | 2 |
| RPA2 | AT2G24490 | DNA replication | N | TAP | + | 2 |
| UVH6 | AT1G03190 | DNA repair | C | TAP | + | 2 |
| BUB3 | AT1G69400 | mitotic checkpoint | N | TAP | + | 2 |
| BUB3-like | AT3G19590 | mitotic checkpoint | N | TAP | + | 2 |
| BUBR1-like | AT2G33560 | mitotic checkpoint | N | TAP | + | 2 |
| MAD2-like | AT3G25980 | mitotic checkpoint | N | TAP | + | 3 |
| DL3195C | AT4G14310 | reverse | N | TAP | + | 2 |
| F27G20.14 | AT1G32310 | reverse | C | GS | + | 2 |
| expressed | AT5G40460 | reverse | C | TAP | + | 2 |
| expressed | AT1G10690 | reverse | N | TAP | + | 2 |
| UVI4 | AT2G42260 | reverse | N | TAP | + | 2 |
| UVI4-like | AT3G57860 | reverse | N | TAP | + | 2 |

TABLE 2
List of control identifications.
Control identifications determined by mock TAP (7) or GS (3)
purifications, and by purifications with extracts from cultures
expressing TAP fusions of heterologous GFP (7), RFP (2) or
β-glucuronidase (5), or GS fusions of heterologous GFP (8),
or β-glucuronidase (4). Ribosomal proteins, actins and tubulins
identified in these control experiments were not included.
The number of control experiments is shown between brackets.

| Accession | |
|---|---|
| number | Protein name |
| AT1G01460 | phosphatidylinositol-4-phosphate 5-kinase family protein |
| AT1G02400 | gibberellin 2-oxidase, putative/GA2-oxidase, putative |
| AT1G02500 | S-adenosylmethionine synthetase 1 (SAM1) |
| AT1G02930 | glutathione S-transferase, putative |
| AT1G04190 | tetratricopeptide repeat (TPR)-containing protein |
| AT1G04600 | myosin, putative |
| AT1G04690 | potassium channel protein, putative |
| AT1G05320 | myosin-related |
| AT1G05450 | protease inhibitor/seed storage/lipid transfer protein |
| | (LTP)-related |
| AT1G05970 | expressed protein |
| AT1G06220 | elongation factor Tu family protein |
| AT1G06780 | glycosyl transferase family 8 protein |
| AT1G07920 | elongation factor 1-alpha |
| AT1G07930 | elongation factor 1-alpha |
| AT1G08520 | magnesium-chelatase subunit chlD, chloroplast, putative |
| AT1G09080 | luminal binding protein 3 (BiP-3) (BP3) |
| AT1G09450 | haspin-related |
| AT1G09780 | 2,3-biphosphoglycerate-independent phosphoglycerate |
| | mutase, putative |
| AT1G09980 | expressed protein |
| AT1G10270 | pentatricopeptide (PPR) repeat-containing protein |
| AT1G10390 | nucleoporin family protein |
| AT1G10590 | DNA-binding protein-related |
| AT1G11480 | eukaryotic translation initiation factor-related |
| AT1G12410 | ATP-dependent Clp protease proteolytic subunit (ClpP2) |
| AT1G13020 | eukaryotic translation initiation factor, putative |
| AT1G13440 | glyceraldehyde 3-phosphate dehydrogenase, cytosolic, |
| | putative |
| AT1G13610 | expressed protein |
| AT1G13910 | F16A14.12 (leucine-rich repeat family protein) |
| AT1G14315 | Putative F-box protein |
| AT1G14850 | non-repetitive/WGA-negative nucleoporin family protein |
| AT1G14980 | 10 kDa chaperonin (CPN10) |
| AT1G15730 | PRLI-interacting factor L, putative (Fragment) |
| AT1G16030 | heat shock protein 70, putative |
| AT1G16410 | cytochrome P450, putative |
| AT1G16750 | expressed protein |
| AT1G18370 | kinesin motor family protein (NACK1) |
| AT1G19290 | pentatricopeptide (PPR) repeat-containing protein |
| AT1G19920 | sulfate adenylyltransferase 2/ATP-sulfurylase 2 |
| AT1G21480 | exostosin family protein |
| AT1G23410 | ubiquitin extension protein, putative/40S ribosomal protein |
| | S27A |
| AT1G24300 | GYF domain-containing protein |
| AT1G24510 | T-complex protein 1 epsilon subunit, putative |
| AT1G24938 | hypothetical protein |
| AT1G26750 | expressed protein |
| AT1G27430 | GYF domain-containing protein |
| AT1G27970 | nuclear transport factor 2 (NTF2), putative |
| AT1G29350 | kinase-related |
| AT1G29370 | kinase-related |
| AT1G31230 | bifunctional aspartate kinase/homoserine dehydrogenase |
| AT1G31280 | PAZ domain-containing protein/piwi domain-containing |
| | protein (Fragment) |
| AT1G31770 | ABC transporter family protein |
| AT1G34610 | Ulp1 protease family protein |
| AT1G35380 | hypothetical protein |
| AT1G36120 | putative reverse transcriptase |
| AT1G36280 | adenylosuccinate lyase, putative/adenylosuccinase, putative |
| AT1G36580 | 2,4-dienoyl-CoA reductase-related |
| AT1G37200 | gypsy-like retrotransposon family protein |
| AT1G44900 | DNA replication licensing factor, putative, similar to DNA |
| | replication licensing factor MCM2 |
| AT1G48400 | F-box family protein |
| AT1G48630 | guanine nucleotide-binding family protein/activated protein |
| | kinase |
| AT1G49040 | stomatal cytokinesis defective/SCD1 protein (SCD1) |
| AT1G51380 | eukaryotic translation initiation factor 4A, putative |
| AT1G51710 | ubiquitin-specific protease 6, putative (UBP6) |
| AT1G52450 | ubiquitin carboxyl-terminal hydrolase-related |
| AT1G52740 | histone H2A, putative |
| AT1G53240 | malate dehydrogenase [NAD], mitochondrial, putative |
| AT1G53550 | F-box family protein |
| AT1G54270 | eukaryotic translation initiation factor 4A-2 |
| AT1G55490 | RuBisCO subunit binding-protein beta subunit, chloroplast |
| AT1G56070 | elongation factor 2, putative |
| AT1G56070 | elongation factor 2, putative |
| AT1G56410 | heat shock cognate 70 kDa protein, putative |
| AT1G58265 | cytochrome P450-related |
| AT1G59610 | dynamin-like protein, putative (ADL3) |
| AT1G61210 | WD-40 repeat family protein/katanin p80 subunit, putative |
| AT1G61300 | disease resistance protein (NBS-LRR class), putative |
| AT1G61570 | mitochondrial import inner membrane translocase (TIM13) |
| AT1G62020 | coatomer protein complex, subunit alpha, putative |
| AT1G62610 | short-chain dehydrogenase/reductase (SDR) family protein |
| AT1G62630 | disease resistance protein (CC-NBS-LRR class), putative |
| AT1G64520 | 26S proteasome regulatory subunit, putative (RPN12) |
| AT1G64550 | ABC transporter family protein |
| AT1G65130 | ubiquitin carboxyl-terminal hydrolase-related |
| AT1G65290 | acyl carrier family protein/ACP family protein |
| AT1G65540 | calcium-binding EF hand family protein |
| AT1G65870 | disease resistance-responsive family protein |
| AT1G66410 | calmodulin |
| AT1G66510 | AAR2 protein family |
| AT1G67090 | ribulose bisphosphate carboxylase small chain 1A |
| AT1G68750 | phosphoenolpyruvate carboxylase family protein |
| AT1G68910 | expressed protein similar to Myosin heavy chain, |
| | nonmuscle type B |
| AT1G70100 | expressed protein |
| AT1G71220 | UDP-glucose:glycoprotein glucosyltransferase, putative |
| AT1G71270 | Vps52/Sac2 family protein |
| AT1G72000 | beta-fructofuranosidase, putative/invertase, putative/ |
| | saccharase, putative |
| AT1G72730 | eukaryotic translation initiation factor 4A, putative |
| AT1G73720 | transducin family protein/WD-40 repeat family protein |
| AT1G75010 | MORN (Membrane Occupation and Recognition Nexus) |
| | repeat-containing protein |
| AT1G76520 | auxin efflux carrier family protein |
| AT1G76530 | auxin efflux carrier family protein |
| AT1G77120 | alcohol dehydrogenase (ADH) |
| AT1G78130 | transporter-related low similarity to spinster type III |
| AT1G78390 | 9-cis-epoxycarotenoid dioxygenase, putative |
| AT1G78900 | vacuolar ATP synthase catalytic subunit A |
| AT1G79280 | expressed protein |
| AT1G79550 | phosphoglycerate kinase, putative |
| AT1G79920 | heat shock protein 70, putative |
| AT1G79930 | heat shock protein, putative |
| AT1G80070 | splicing factor, putative |
| AT1G80270 | DNA-binding protein, putative |
| AT1G80670 | transducin family protein/WD-40 repeat family protein |
| AT2G01210 | leucine-rich repeat transmembrane protein kinase, putative |
| AT2G01350 | quinolinate phosphoribosyl transferase family protein |
| AT2G02160 | zinc finger (CCCH-type) family protein |
| AT2G03430 | ankyrin repeat family protein |
| AT2G04030 | heat shock protein, putative |
| AT2G05990 | enoyl-[acyl-carrier protein] reductase [NADH], chloroplast, |
| | putative |
| AT2G06850 | xyloglucan:xyloglucosyl transferase |
| AT2G07620 | putative helicase |
| AT2G07714 | transcription factor-related similar to male sterility 1 |
| | proteins |
| AT2G13680 | callose synthase 5 (1,3-beta-glucan synthase) |
| AT2G14120 | dynamin-like protein 2b |
| AT2G14880 | SWIB complex BAF60b domain-containing protein |
| AT2G16600 | peptidyl-prolyl cis-trans isomerase, cytosolic/cyclophilin |
| AT2G17130 | isocitrate dehydrogenase subunit 2/NAD+ isocitrate |
| | dehydrogenase subunit 2 |
| AT2G18700 | glycosyl transferase family 20 protein |
| AT2G19070 | transferase family protein |
| AT2G19210 | leucine-rich repeat protein kinase, putative |
| AT2G20330 | transducin family protein/WD-40 repeat family protein |
| AT2G20420 | succinyl-CoA ligase [GDP-forming] beta-chain, |
| | mitochondrial, putative |
| AT2G20580 | 26S proteasome regulatory subunit S2 (RPN1) |
| AT2G20805 | hypothetical protein |
| AT2G21170 | triosephosphate isomerase, chloroplast, putative |
| AT2G21390 | coatomer protein complex, subunit alpha, putative |
| AT2G21410 | vacuolar proton ATPase, putative |
| AT2G21920 | hypothetical protein (Fragment) |
| AT2G22230 | beta-hydroxyacyl-ACP dehydratase, putative |
| AT2G23930 | small nuclear ribonucleoprotein G, putative |
| AT2G24420 | DNA repair ATPase-related contains 2 transmembrane |
| | domains |
| AT2G26570 | expressed protein |
| AT2G26890 | DNAJ heat shock N-terminal domain-containing protein |
| AT2G27030 | calmodulin |
| AT2G27610 | pentatricopeptide (PPR) repeat-containing protein |
| AT2G27870 | hypothetical protein |
| AT2G28000 | RuBisCO subunit binding-protein alpha subunit, chloroplast |
| AT2G28620 | kinesin motor protein-related |
| AT2G29540 | DNA-directed RNA polymerase I(A) and III(C) 14 kDa |
| | subunit (RPAC14) |
| AT2G31680 | Ras-related GTP-binding protein, putative |
| AT2G32240 | putative myosin heavy chain |
| AT2G33210 | chaperonin, putative |
| AT2G33730 | DEAD box RNA helicase, putative |
| AT2G34170 | expressed protein |
| AT2G35605 | SWIB complex BAF60b domain-containing protein |
| AT2G36060 | ubiquitin-conjugating enzyme family protein |
| AT2G36260 | iron-sulfur cluster assembly complex protein, putative |
| AT2G36460 | fructose-bisphosphate aldolase, putative |
| AT2G36810 | expressed protein |
| AT2G37230 | pentatricopeptide (PPR) repeat-containing protein |
| AT2G37420 | kinesin motor protein-related |
| AT2G38560 | transcription factor S-II (TFIIS) domain-containing protein |
| AT2G38810 | histone H2A, putative |
| AT2G39730 | ribulose bisphosphate carboxylase/oxygenase activase |
| AT2G39990 | eukaryotic translation initiation factor 3 subunit 5 |
| AT2G40070 | expressed protein |
| AT2G40270 | protein kinase family protein |
| AT2G41450 | GCN5-related N-acetyltransferase (GNAT) family protein |
| AT2G41800 | expressed protein |
| AT2G42230 | tubulin-specific chaperone C-related |
| AT2G42450 | lipase class 3 family protein |
| AT2G42520 | DEAD box RNA helicase, putative |
| AT2G43160 | epsin N-terminal homology (ENTH) domain-containing |
| | protein |
| AT2G43750 | cysteine synthase, chloroplast/O-acetylserine (thiol)-lyase |
| AT2G44060 | late embryogenesis abundant family protein/LEA family |
| | protein |
| AT2G45030 | mitochondrial elongation factor, putative |
| AT2G45620 | nucleotidyltransferase family protein |
| AT2G46520 | cellular apoptosis susceptibility protein, putative/importin- |
| | alpha |
| AT2G47110 | ubiquitin extension protein 6 (UBQ6)/40S ribosomal protein |
| | S27A |
| AT3G01040 | glycosyl transferase family 8 protein |
| AT3G01720 | expressed protein |
| AT3G01740 | expressed protein |
| AT3G02200 | proteasome family protein |
| AT3G02230 | reversibly glycosylated polypeptide-1 |
| AT3G02500 | unknown protein |
| AT3G02530 | chaperonin, putative |
| AT3G02650 | pentatricopeptide (PPR) repeat-containing protein |
| AT3G03300 | DEAD/DEAH box helicase carpel factory-related |
| AT3G03780 | AtMS2\|AtMS2 (Arabidopsis thaliana methionine |
| | synthase 2) |
| AT3G03960 | chaperonin, putative |
| AT3G04120 | glyceraldehyde-3-phosphate dehydrogenase, cytosolic |
| AT3G05810 | expressed protein |
| AT3G07160 | glycosyl transferase family 48 protein |
| AT3G07630 | prephenate dehydratase family protein |
| AT3G07810 | heterogeneous nuclear ribonucleoprotein, putative |
| AT3G08030 | expressed protein |
| AT3G08530 | clathrin heavy chain, putative |
| AT3G08590 | 2,3-biphosphoglycerate-independent phosphoglycerate |
| | mutase, putative |
| AT3G09170 | Ulp1 protease family protein |
| AT3G09350 | armadillo/beta-catenin repeat family protein |
| AT3G09440 | heat shock cognate 70 kDa protein 3 |
| AT3G10310 | kinesin motor protein-related |
| AT3G10860 | ubiquinol-cytochrome C reductase complex ubiquinone- |
| | binding protein |
| AT3G11130 | clathrin heavy chain, putative |
| AT3G11400 | eukaryotic translation initiation factor 3G |
| AT3G11770 | expressed protein |
| AT3G11950 | ATHST; prenyltransferase |
| AT3G12580 | heat shock protein 70, putative |
| AT3G12800 | short-chain dehydrogenase/reductase (SDR) family protein |
| AT3G13160 | pentatricopeptide (PPR) repeat-containing protein |
| AT3G13460 | expressed protein |
| AT3G13470 | chaperonin, putative |
| AT3G13920 | eukaryotic translation initiation factor 4A-1 |
| AT3G14990 | 4-methyl-5(b-hydroxyethyl)-thiazole monophosphate |
| | biosynthesis protein |
| AT3G15020 | malate dehydrogenase [NAD], mitochondrial, putative |
| AT3G16230 | expressed protein similar to ASC-1 complex subunit P50 |
| AT3G16270 | expressed protein |
| AT3G16540 | DegP protease, putative |
| AT3G16640 | translationally controlled tumor family protein |
| AT3G17300 | expressed protein |
| AT3G17360 | kinesin motor protein-related similar to KLP2 protein |
| AT3G17390 | S-adenosylmethionine synthetase, putative |
| AT3G18000 | phosphoethanolamine N-methyltransferase 1 |
| AT3G18190 | chaperonin, putative |
| AT3G18530 | expressed protein |
| AT3G19050 | kinesin motor protein-related |
| AT3G20670 | histone H2A, putative |
| AT3G20780 | topoisomerase 6 subunit B (TOP6B) |
| AT3G21070 | ATP-NAD kinase family protein |
| AT3G22330 | DEAD box RNA helicase, putative |
| AT3G22890 | sulfate adenylyltransferase 1/ATP-sulfurylase 1 |
| AT3G23990 | chaperonin (CPN60) (HSP60) |
| AT3G24890 | synaptobrevin-related |
| AT3G26020 | serine/threonine protein phosphatase 2A (PP2A) regulatory |
| | subunit B′, putative |
| AT3G26380 | glycosyl hydrolase family protein 27 |
| AT3G27400 | pectate lyase family protein |
| AT3G28925 | hypothetical protein |
| AT3G42100 | AT hook motif-containing protein-related |
| AT3G42270 | putative protein |
| AT3G43300 | guanine nucleotide exchange family protein |
| AT3G43810 | calmodulin |
| AT3G45770 | oxidoreductase, zinc-binding dehydrogenase family protein |
| AT3G46560 | mitochondrial import inner membrane translocase (TIM9) |
| AT3G46990 | hypothetical protein |
| AT3G47850 | expressed protein |
| AT3G48250 | pentatricopeptide (PPR) repeat-containing protein |
| AT3G48670 | XH/XS domain-containing protein/XS zinc finger domain- |
| | containing protein |
| AT3G48870 | ATP-dependent Clp protease ATP-binding subunit (ClpC) |
| AT3G49640 | nitrogen regulation family protein |
| AT3G50280 | transferase family protein |
| AT3G50370 | expressed protein |
| AT3G51150 | kinesin motor family protein |
| AT3G51570 | disease resistance protein (TIR-NBS-LRR class), putative |
| AT3G51950 | zinc finger (CCCH-type) family protein/RNA recognition |
| | motif (RRM) |
| AT3G52140 | tetratricopeptide repeat (TPR)-containing protein |
| AT3G52930 | fructose-bisphosphate aldolase, putative |
| AT3G54360 | expressed protein DNA-binding Mel-18 protein |
| AT3G54610 | histone acetyltransferase (GCN5) |
| AT3G54940 | cysteine proteinase, putative |
| AT3G55000 | Tonneau 1a |
| AT3G55005 | Tonneau 1b |
| AT3G55290 | short-chain dehydrogenase/reductase (SDR) family protein |
| AT3G55310 | short-chain dehydrogenase/reductase (SDR) family protein |
| AT3G55460 | SC35-like splicing factor, 30 kD (SCL30) |
| AT3G55770 | LIM domain-containing protein |
| AT3G57290 | eukaryotic translation initiation factor 3E |
| AT3G58510 | DEAD box RNA helicase, putative (RH11) |
| AT3G58610 | ketol-acid reductoisomerase |
| AT3G59760 | cysteine synthase, mitochondrial, putative |
| AT3G62010 | expressed protein |
| AT3G62250 | ubiquitin extension protein 5 (UBQ5)/40S ribosomal |
| | protein S27A |
| AT4G00020 | BRCA2 repeat-containing protein |
| AT4G01290 | expressed protein |
| AT4G01800 | preprotein translocase secA subunit, putative |
| AT4G02930 | elongation factor Tu, putative |
| AT4G03550 | glycosyl transferase family 48 protein |
| AT4G11150 | vacuolar ATP synthase subunit E |
| AT4G11420 | eukaryotic translation initiation factor 3 subunit 10 |
| AT4G12130 | glycine cleavage T family protein |
| AT4G12770 | auxilin-related |
| AT4G12780 | auxilin-like protein |
| AT4G14060 | major latex protein-related |
| AT4G14130 | xyloglucan:xyloglucosyl transferase, putative |
| AT4G14140 | DNA (cytosine-5-)-methyltransferase (METII) |
| AT4G14260 | hypothetical protein |
| AT4G15640 | expressed protein |
| AT4G15760 | monooxygenase, putative (MO1) |
| AT4G17150 | unknown protein |
| AT4G17440 | expressed protein |
| AT4G17720 | RNA recognition motif (RRM)-containing protein |
| AT4G18040 | eukaryotic translation initiation factor 4E 1 |
| AT4G18080 | hypothetical protein |
| AT4G19530 | disease resistance protein (TIR-NBS-LRR class), putative |
| AT4G20160 | expressed protein |
| AT4G20360 | elongation factor Tu |
| AT4G20980 | eukaryotic translation initiation factor 3 subunit 7, putative |
| AT4G23670 | major latex protein-related |
| AT4G23850 | long-chain-fatty-acid--CoA ligase/long-chain acyl-CoA |
| | synthetase |
| AT4G24100 | protein kinase family protein |
| AT4G24280 | heat shock protein 70, putative |
| AT4G24760 | expressed protein |
| AT4G25580 | stress-responsive protein-related |
| AT4G26900 | imidazole glycerol phosphate synthase hisHF, chloroplast |
| AT4G27440 | protochlorophyllide reductase B, chloroplast |
| AT4G28400 | protein phosphatase 2C, putative |
| AT4G29800 | patatin-related low similarity to patatin precursor |
| AT4G31805 | WRKY family transcription factor |
| AT4G31820 | phototropic-responsive NPH3 family protein |
| AT4G31990 | aspartate aminotransferase, chloroplast/transaminase A |
| | (ASP5) (AAT1) |
| AT4G32500 | potassium channel protein, putative |
| AT4G32551 | WD-40 repeat family protein (LEUNIG) |
| AT4G33200 | myosin, putative |
| AT4G34290 | SWIB complex BAF60b domain-containing protein |
| AT4G34350 | LytB family protein |
| AT4G35260 | isocitrate dehydrogenase subunit 1/NAD+ isocitrate |
| | dehydrogenase subunit 1 |
| AT4G35650 | isocitrate dehydrogenase, putative/NAD+ isocitrate |
| | dehydrogenase, putative |
| AT4G35890 | La domain-containing protein |
| AT4G35970 | L-ascorbate peroxidase, putative |
| AT4G36960 | RNA recognition motif (RRM)-containing protein |
| AT4G37420 | hypothetical protein |
| AT4G37580 | N-acetyltransferase, putative/hookless1 (HLS1) |
| AT4G37910 | heat shock protein 70, mitochondrial, putative |
| AT4G38030 | hypothetical protein |
| AT4G38070 | bHLH family protein |
| AT4G39580 | kelch repeat-containing F-box family protein |
| AT5G01010 | expressed protein |
| AT5G02490 | heat shock cognate 70 kDa protein 2 |
| AT5G02500 | heat shock cognate 70 kDa protein 1 |
| AT5G02590 | chloroplast lumen common family protein |
| AT5G03340 | cell division cycle protein 48, putative |
| AT5G03650 | 1,4-alpha-glucan branching enzyme |
| AT5G03690 | fructose-bisphosphate aldolase, putative |
| AT5G04590 | sulfite reductase/ferredoxin |
| AT5G05370 | ubiquinol-cytochrome C reductase complex ubiquinone- |
| | binding protein |
| AT5G05640 | nucleoprotein-related |
| AT5G05780 | 26S proteasome non-ATPase regulatory subunit 7, putative |
| AT5G06140 | phox (PX) domain-containing protein |
| AT5G06400 | pentatricopeptide (PPR) repeat-containing protein |
| AT5G06450 | expressed protein |
| AT5G06850 | anthranilate phosphoribosyltransferase-like protein |
| AT5G08415 | lipoic acid synthase family protein |
| AT5G08670 | ATP synthase beta chain, mitochondrial |
| AT5G08680 | ATP synthase beta chain, mitochondrial |
| AT5G08690 | ATP synthase beta chain, mitochondrial |
| AT5G09590 | heat shock protein 70 |
| AT5G09900 | 26S proteasome regulatory subunit, putative (RPN5) |
| AT5G10160 | beta-hydroxyacyl-ACP dehydratase, putative |
| AT5G11110 | sucrose-phosphate synthase-like protein |
| AT5G11340 | GCN5-related N-acetyltransferase (GNAT) family protein |
| AT5G12010 | expressed protein |
| AT5G12240 | expressed protein |
| AT5G12310 | zinc finger (C3HC4-type RING finger) family protein |
| AT5G13130 | hypothetical protein low similarity to microrchidia |
| AT5G14040 | mitochondrial phosphate transporter |
| AT5G15580 | expressed protein |
| AT5G15610 | proteasome family protein |
| AT5G15650 | reversibly glycosylated polypeptide-2 |
| AT5G15920 | structural maintenance of chromosomes (SMC) family |
| | protein |
| AT5G16070 | chaperonin, putative |
| AT5G17680 | disease resistance protein (TIR-NBS-LRR class), putative |
| AT5G17890 | LIM domain-containing protein/disease resistance protein- |
| | related |
| AT5G17920 | 5-methyltetrahydropteroyltriglutamate--homocysteine |
| | methyltransferase |
| AT5G18110 | novel cap-binding protein (nCBP) |
| AT5G18730 | hypothetical protein |
| AT5G19310 | homeotic gene regulator, putative |
| AT5G19550 | aspartate aminotransferase, cytoplasmic isozyme |
| | 1/transaminase A |
| AT5G20010 | Ras-related GTP-binding nuclear protein |
| AT5G20020 | Ras-related GTP-binding nuclear protein |
| AT5G20360 | octicosapeptide/Phox/Bem1p (PB1) domain-containing |
| | protein (Fragment) |
| AT5G20720 | 20 kDa chaperonin, chloroplast |
| AT5G20890 | chaperonin, putative |
| AT5G21274 | calmodulin |
| AT5G22650 | expressed protein |
| AT5G23540 | 26S proteasome regulatory subunit, putative |
| AT5G23890 | expressed protein |
| AT5G23930 | mitochondrial transcription termination factor-related |
| AT5G24710 | WD-40 repeat family protein |
| AT5G25230 | elongation factor Tu family protein |
| AT5G25940 | early nodulin-related |
| AT5G26200 | mitochondrial substrate carrier family protein |
| AT5G26710 | glutamate-tRNA ligase, putative |
| AT5G26742 | DEAD box RNA helicase (RH3) |
| AT5G26800 | expressed protein |
| AT5G27390 | expressed protein |
| AT5G27920 | F-box family protein |
| AT5G28430 | hypothetical protein |
| AT5G28540 | luminal binding protein 1 |
| AT5G28850 | calcium-binding EF hand family protein |
| AT5G35360 | acetyl-CoA carboxylase, biotin carboxylase subunit (CAC2) |
| AT5G35620 | eukaryotic translation initiation factor 4E 2 |
| AT5G36670 | putative protein |
| AT5G37150 | tRNA-splicing endonuclease positive effector-related |
| AT5G37470 | hypothetical protein |
| AT5G37510 | NADH-ubiquinone dehydrogenase, mitochondrial, putative |
| AT5G39320 | UDP-glucose 6-dehydrogenase, putative |
| AT5G40060 | disease resistance protein (TIR-NBS-LRR class), putative |
| AT5G40760 | glucose-6-phosphate 1-dehydrogenase/G6PD |
| AT5G42020 | luminal binding protein 2 |
| AT5G42080 | GTP-binding protein/phragmoplastin, putative |
| AT5G42950 | GYF domain-containing protein |
| AT5G42980 | thioredoxin H-type 3 |
| AT5G43780 | sulfate adenylyltransferase 4/ATP-sulfurylase 4 |
| AT5G44320 | eukaryotic translation initiation factor 3 subunit 7, putative |
| AT5G44510 | disease resistance protein (TIR-NBS-LRR class), putative |
| | (TAO1) |
| AT5G45060 | disease resistance protein (TIR-NBS-LRR class), putative |
| AT5G45140 | DNA-directed RNA polymerase, putative |
| AT5G47460 | pentatricopeptide (PPR) repeat-containing protein |
| AT5G47890 | NADH-ubiquinone oxidoreductase B8 subunit, putative |
| AT5G48620 | disease resistance protein (CC-NBS-LRR class), putative |
| | (Fragment) |
| AT5G49030 | tRNA synthetase class I (I, L, M and V) family protein |
| AT5G49910 | heat shock protein 70 |
| AT5G50370 | adenylate kinase, putative |
| AT5G50810 | mitochondrial import inner membrane translocase (TIM8) |
| AT5G50920 | ATP-dependent Clp protease ATP-binding subunit (ClpC) |
| AT5G51000 | F-box family protein |
| AT5G51670 | expressed protein |
| AT5G51795 | Kin17 DNA-binding protein-related (Fragment) |
| AT5G52040 | arginine/serine-rich splicing factor RSP41 (RSP41) |
| AT5G52400 | cytochrome P450 family protein |
| AT5G53460 | glutamate synthase [NADH], chloroplast, putative |
| AT5G54640 | histone H2A |
| AT5G54670 | kinesin-like protein C (KATC) |
| AT5G55190 | Ras-related GTP-binding nuclear protein |
| AT5G56000 | heat shock protein 81 |
| AT5G56030 | heat shock protein 81 |
| AT5G56500 | chaperonin, putative |
| AT5G57350 | ATPase 3, plasma membrane-type/proton pump 3 |
| AT5G58070 | lipocalin, putative similar to temperature stress-induced |
| | lipocalin |
| AT5G59220 | protein phosphatase 2C, putative |
| AT5G59620 | putative protein |
| AT5G59880 | actin-depolymerizing factor 3 (ADF3) |
| AT5G60390 | elongation factor 1-alpha |
| AT5G60720 | expressed protein |
| AT5G60980 | nuclear transport factor 2 (NTF2) family protein |
| AT5G61780 | tudor domain-containing protein/nuclease family protein |
| AT5G63020 | disease resistance protein (CC-NBS-LRR class), putative |
| AT5G63400 | adenylate kinase |
| AT5G63800 | glycosyl hydrolase family 35 protein |
| AT5G64760 | 26S proteasome regulatory subunit, putative (RPN5) |
| AT5G66470 | expressed protein |

TABLE 3
Overrepresentation of Biological Processes among the preys of the core (A) and
non-core (B) dataset as determined with the Cytoscape plugin BiNGO.
A. Overrepresentation of Biological Processes among the preys of the core dataset.

Search parameters:
File created with BiNGO (c) on 22-okt-2008 at 16:17:17
ontology: process
curator: GO
Selected ontology file: BiNGO.jar!/GO_Biological_Process
Selected annotation file: gene_association_2008okt04.tair
Overrepresentation
Selected statistical test: Hypergeometric test
Selected correction: Benjamini & Hochberg False Discovery Rate (FDR) correction
Selected significance level: 0.05
Testing option: Test cluster versus whole annotation
Number of annotated genes in selection: 137
Number of annotated genes in network/whole annotation: 25557

Results:

| | | corr | # | | |
|---|---|---|---|---|---|
| GO-ID | p-value | p-value | selected | # total | Description |
| 7049 | 2.30E−34 | 7.17E−32 | 28 | 164 | cell cycle |
| 51726 | 1.11E−28 | 1.74E−26 | 21 | 93 | regulation of cell cycle |
| 6260 | 2.09E−18 | 2.17E−16 | 16 | 116 | DNA replication |
| 6263 | 4.33E−18 | 3.38E−16 | 13 | 58 | DNA-dependent DNA replication |
| 74 | 8.10E−15 | 5.05E−13 | 9 | 26 | regulation of progression through cell cycle |
| 6259 | 7.00E−14 | 3.60E−12 | 21 | 463 | DNA metabolism |
| 43285 | 8.43E−14 | 3.60E−12 | 18 | 312 | biopolymer catabolism |
| 30163 | 9.23E−14 | 3.60E−12 | 17 | 268 | protein catabolism |
| 9057 | 3.38E−13 | 1.10E−11 | 19 | 390 | macromolecule catabolism |
| 6511 | 4.22E−13 | 1.10E−11 | 16 | 249 | ubiquitin-dependent protein catabolism |
| 43632 | 4.22E−13 | 1.10E−11 | 16 | 249 | modification-dependent macromolecule catabolism |
| 19941 | 4.22E−13 | 1.10E−11 | 16 | 249 | modification-dependent protein catabolism |
| 51603 | 4.77E−13 | 1.15E−11 | 16 | 251 | proteolysis during cellular protein catabolism |
| 44257 | 6.86E−13 | 1.53E−11 | 16 | 257 | cellular protein catabolism |
| 44265 | 1.05E−12 | 2.19E−11 | 18 | 362 | cellular macromolecule catabolism |
| 79 | 4.38E−12 | 8.55E−11 | 6 | 10 | regulation of cyclin-dependent protein kinase activity |
| 6270 | 1.91E−11 | 3.51E−10 | 6 | 12 | DNA replication initiation |
| 278 | 3.69E−11 | 6.40E−10 | 8 | 40 | mitotic cell cycle |
| 6469 | 2.27E−10 | 3.38E−09 | 5 | 8 | negative regulation of protein kinase activity |
| 45736 | 2.27E−10 | 3.38E−09 | 5 | 8 | negative regulation of cyclin-dependent protein kinase activity |
| 51348 | 2.27E−10 | 3.38E−09 | 5 | 8 | negative regulation of transferase activity |
| 8151 | 2.45E−10 | 3.47E−09 | 97 | 11293 | cellular physiological process |
| 9056 | 3.36E−10 | 4.55E−09 | 19 | 582 | catabolism |
| 45786 | 5.09E−10 | 6.62E−09 | 5 | 9 | negative regulation of progression through cell cycle |
| 42023 | 5.44E−10 | 6.79E−09 | 6 | 19 | DNA endoreduplication |
| 43086 | 1.01E−09 | 1.22E−08 | 5 | 10 | negative regulation of enzyme activity |
| 44248 | 1.30E−09 | 1.50E−08 | 18 | 560 | cellular catabolism |
| 43283 | 1.38E−09 | 1.54E−08 | 48 | 3695 | biopolymer metabolism |
| 45859 | 1.99E−09 | 2.04E−08 | 6 | 23 | regulation of protein kinase activity |
| 43549 | 1.99E−09 | 2.04E−08 | 6 | 23 | regulation of kinase activity |
| 9987 | 2.03E−09 | 2.04E−08 | 98 | 11858 | cellular process |
| 51338 | 2.64E−09 | 2.58E−08 | 6 | 24 | regulation of transferase activity |
| 50794 | 6.88E−09 | 6.51E−08 | 33 | 2040 | regulation of cellular process |
| 51325 | 7.92E−09 | 7.06E−08 | 5 | 14 | interphase |
| 51329 | 7.92E−09 | 7.06E−08 | 5 | 14 | interphase of mitotic cell cycle |
| 51244 | 1.26E−08 | 1.09E−07 | 32 | 1980 | regulation of cellular physiological process |
| 50791 | 3.60E−08 | 3.04E−07 | 32 | 2070 | regulation of physiological process |
| 7582 | 1.33E−07 | 1.09E−06 | 100 | 13066 | physiological process |
| 50789 | 2.21E−07 | 1.77E−06 | 33 | 2359 | regulation of biological process |
| 50790 | 2.72E−07 | 2.12E−06 | 7 | 81 | regulation of catalytic activity |
| 6139 | 6.29E−07 | 4.79E−06 | 37 | 2974 | nucleobase, nucleoside, nucleotide and nucleic acid |
| | | | | | metabolism |
| 43170 | 1.23E−06 | 9.17E−06 | 60 | 6396 | macromolecule metabolism |
| 6508 | 1.61E−06 | 1.17E−05 | 16 | 718 | proteolysis |
| 51302 | 1.24E−05 | 8.77E−05 | 3 | 9 | regulation of cell division |
| 44238 | 1.87E−05 | 1.30E−04 | 69 | 8433 | primary metabolism |
| 51510 | 2.85E−05 | 1.93E−04 | 2 | 2 | regulation of unidimensional cell growth |
| 44237 | 1.45E−04 | 9.61E−04 | 67 | 8584 | cellular metabolism |
| 80 | 1.70E−04 | 1.06E−03 | 2 | 4 | G1 phase of mitotic cell cycle |
| 82 | 1.70E−04 | 1.06E−03 | 2 | 4 | G1/S transition of mitotic cell cycle |
| 51318 | 1.70E−04 | 1.06E−03 | 2 | 4 | G1 phase |
| 7346 | 2.82E−04 | 1.73E−03 | 2 | 5 | regulation of progression through mitotic cell cycle |
| 48856 | 3.80E−04 | 2.28E−03 | 17 | 1248 | anatomical structure development |
| 48519 | 4.57E−04 | 2.69E−03 | 6 | 182 | negative regulation of biological process |
| 9653 | 5.58E−04 | 3.22E−03 | 10 | 526 | morphogenesis |
| 51243 | 6.65E−04 | 3.72E−03 | 5 | 129 | negative regulation of cellular physiological process |
| 51301 | 6.67E−04 | 3.72E−03 | 3 | 32 | cell division |
| 48523 | 6.89E−04 | 3.77E−03 | 5 | 130 | negative regulation of cellular process |
| 1558 | 7.82E−04 | 4.21E−03 | 2 | 8 | regulation of cell growth |
| 43118 | 1.02E−03 | 5.42E−03 | 5 | 142 | negative regulation of physiological process |
| 6281 | 1.09E−03 | 5.67E−03 | 5 | 144 | DNA repair |
| 9793 | 1.28E−03 | 6.56E−03 | 9 | 487 | embryonic development (sensu Magnoliophyta) |
| 6974 | 1.47E−03 | 7.39E−03 | 5 | 154 | response to DNA damage stimulus |
| 9790 | 2.01E−03 | 9.94E−03 | 9 | 520 | embryonic development |
| 48316 | 2.28E−03 | 1.11E−02 | 9 | 530 | seed development |
| 48272 | 2.32E−03 | 1.11E−02 | 3 | 49 | trichome morphogenesis (sensu Magnoliophyta) |
| 48608 | 2.37E−03 | 1.12E−02 | 9 | 533 | reproductive structure development |
| 7148 | 2.43E−03 | 1.13E−02 | 6 | 252 | cell morphogenesis |
| 6457 | 2.58E−03 | 1.18E−02 | 6 | 255 | protein folding |
| 904 | 2.60E−03 | 1.18E−02 | 3 | 51 | cellular morphogenesis during differentiation |
| 7017 | 2.66E−03 | 1.19E−02 | 4 | 107 | microtubule-based process |
| 7275 | 2.81E−03 | 1.23E−02 | 19 | 1765 | development |
| 9965 | 2.94E−03 | 1.27E−02 | 4 | 110 | leaf morphogenesis |
| 7018 | 3.23E−03 | 1.37E−02 | 3 | 55 | microtubule-based movement |
| 9934 | 3.26E−03 | 1.37E−02 | 2 | 16 | regulation of meristem organization |
| 8152 | 3.29E−03 | 1.37E−02 | 70 | 10044 | metabolism |
| 19952 | 3.72E−03 | 1.53E−02 | 11 | 794 | reproduction |
| 48827 | 3.90E−03 | 1.58E−02 | 5 | 193 | phyllome development |
| 16043 | 3.95E−03 | 1.58E−02 | 16 | 1420 | cell organization and biogenesis |
| 9117 | 4.64E−03 | 1.83E−02 | 4 | 125 | nucleotide metabolism |
| 35315 | 4.95E−03 | 1.91E−02 | 3 | 64 | hair cell differentiation |
| 10026 | 4.95E−03 | 1.91E−02 | 3 | 64 | trichome differentiation (sensu Magnoliophyta) |
| 51446 | 5.36E−03 | 2.02E−02 | 1 | 1 | positive regulation of progression through meiotic cell cycle |
| 51445 | 5.36E−03 | 2.02E−02 | 1 | 1 | regulation of progression through meiotic cell cycle |
| 30154 | 5.56E−03 | 2.07E−02 | 5 | 210 | cell differentiation |
| 9887 | 5.67E−03 | 2.08E−02 | 5 | 211 | organ morphogenesis |
| 30705 | 6.86E−03 | 2.49E−02 | 3 | 72 | cytoskeleton-dependent intracellular transport |
| 48468 | 8.86E−03 | 3.18E−02 | 3 | 79 | cell development |
| 10016 | 8.97E−03 | 3.18E−02 | 4 | 151 | shoot morphogenesis |
| 7276 | 9.80E−03 | 3.44E−02 | 4 | 155 | gametogenesis |
| 7050 | 1.07E−02 | 3.51E−02 | 1 | 2 | cell cycle arrest |
| 42276 | 1.07E−02 | 3.51E−02 | 1 | 2 | error-prone postreplication DNA repair |
| 86 | 1.07E−02 | 3.51E−02 | 1 | 2 | G2/M transition of mitotic cell cycle |
| 45020 | 1.07E−02 | 3.51E−02 | 1 | 2 | error-prone DNA repair |
| 184 | 1.07E−02 | 3.51E−02 | 1 | 2 | mRNA catabolism, nonsense-mediated decay |
| 6358 | 1.07E−02 | 3.51E−02 | 1 | 2 | regulation of global transcription from RNA polymerase II |
| | | | | | promoter |
| 46907 | 1.10E−02 | 3.58E−02 | 7 | 451 | intracellular transport |
| 9165 | 1.15E−02 | 3.70E−02 | 3 | 87 | nucleotide biosynthesis |
| 51649 | 1.18E−02 | 3.75E−02 | 7 | 457 | establishment of cellular localization |
| 51641 | 1.27E−02 | 3.93E−02 | 7 | 464 | cellular localization |
| 9744 | 1.27E−02 | 3.93E−02 | 2 | 32 | response to sucrose stimulus |
| 40008 | 1.27E−02 | 3.93E−02 | 2 | 32 | regulation of growth |
| 48366 | 1.59E−02 | 4.76E−02 | 4 | 179 | leaf development |
| 16288 | 1.60E−02 | 4.76E−02 | 2 | 36 | cytokinesis |
| 10071 | 1.60E−02 | 4.76E−02 | 1 | 3 | root meristem specification |
| 48367 | 1.60E−02 | 4.76E−02 | 5 | 273 | shoot development |

B. Overrepresentation of Biological Processes among the preys of the non-core dataset.

Search parameters:
File created with BiNGO (c) on 22-okt-2008 at 16:28:37
ontology: process
curator: GO
Selected ontology file: BiNGO.jar!/GO_Biological_Process
Selected annotation file: gene_association_2008okt04.tair
Overrepresentation
Selected statistical test: Hypergeometric test
Selected correction: Benjamini & Hochberg False Discovery Rate (FDR) correction
Selected significance level: 0.05
Testing option: Test cluster versus whole annotation
Number of annotated genes in selection: 197
Number of annotated genes in network/whole annotation: 25538

Results:

| | | corr | # | | |
|---|---|---|---|---|---|
| GO-ID | p-value | p-value | selected | # total | Description |
| 46686 | 5.78E−13 | 3.14E−10 | 22 | 396 | response to cadmium ion |
| 10038 | 4.07E−12 | 1.11E−09 | 22 | 437 | response to metal ion |
| 10035 | 9.76E−12 | 1.77E−09 | 22 | 457 | response to inorganic substance |
| 7049 | 4.69E−11 | 6.38E−09 | 14 | 166 | cell cycle |
| 51726 | 2.75E−09 | 2.99E−07 | 10 | 93 | regulation of cell cycle |
| 42221 | 1.01E−08 | 9.16E−07 | 38 | 1789 | response to chemical stimulus |
| 6259 | 2.22E−08 | 1.73E−06 | 18 | 462 | DNA metabolism |
| 8151 | 8.57E−08 | 5.83E−06 | 124 | 11291 | cellular physiological process |
| 50896 | 1.42E−07 | 8.56E−06 | 56 | 3617 | response to stimulus |
| 9987 | 2.38E−07 | 1.29E−05 | 127 | 11856 | cellular process |
| 42023 | 2.76E−07 | 1.36E−05 | 5 | 19 | DNA endoreduplication |
| 6263 | 3.15E−07 | 1.36E−05 | 7 | 58 | DNA-dependent DNA replication |
| 6260 | 3.24E−07 | 1.36E−05 | 9 | 117 | DNA replication |
| 6950 | 8.79E−07 | 3.42E−05 | 28 | 1300 | response to stress |
| 74 | 1.50E−06 | 5.43E−05 | 5 | 26 | regulation of progression through cell cycle |
| 7582 | 2.21E−06 | 7.53E−05 | 133 | 13063 | physiological process |
| 6970 | 2.77E−06 | 8.87E−05 | 14 | 397 | response to osmotic stress |
| 6139 | 6.40E−06 | 1.94E−04 | 45 | 2973 | nucleobase, nucleoside, nucleotide and nucleic acid |
| | | | | | metabolism |
| 9056 | 1.27E−05 | 3.64E−04 | 16 | 582 | catabolism |
| 9628 | 2.56E−05 | 6.96E−04 | 23 | 1140 | response to abiotic stimulus |
| 9651 | 3.21E−05 | 7.96E−04 | 12 | 369 | response to salt stress |
| 44248 | 3.22E−05 | 7.96E−04 | 15 | 560 | cellular catabolism |
| 6454 | 3.40E−05 | 8.04E−04 | 6 | 79 | translational initiation |
| 79 | 5.21E−05 | 1.18E−03 | 3 | 10 | regulation of cyclin-dependent protein kinase activity |
| 9057 | 5.47E−05 | 1.19E−03 | 12 | 390 | macromolecule catabolism |
| 51510 | 5.92E−05 | 1.24E−03 | 2 | 2 | regulation of unidimensional cell growth |
| 44237 | 8.90E−05 | 1.79E−03 | 92 | 8580 | cellular metabolism |
| 15980 | 1.10E−04 | 2.15E−03 | 7 | 140 | energy derivation by oxidation of organic compounds |
| 44265 | 1.25E−04 | 2.35E−03 | 11 | 362 | cellular macromolecule catabolism |
| 45333 | 1.33E−04 | 2.41E−03 | 4 | 34 | cellular respiration |
| 43283 | 1.51E−04 | 2.66E−03 | 48 | 3692 | biopolymer metabolism |
| 9826 | 1.63E−04 | 2.77E−03 | 7 | 149 | unidimensional cell growth |
| 43044 | 1.77E−04 | 2.91E−03 | 2 | 3 | ATP-dependent chromatin remodeling |
| 7275 | 2.23E−04 | 3.56E−03 | 28 | 1765 | development |
| 278 | 2.53E−04 | 3.93E−03 | 4 | 40 | mitotic cell cycle |
| 9266 | 3.13E−04 | 4.74E−03 | 10 | 338 | response to temperature stimulus |
| 9719 | 4.96E−04 | 7.30E−03 | 20 | 1131 | response to endogenous stimulus |
| 6084 | 5.43E−04 | 7.77E−03 | 3 | 21 | acetyl-CoA metabolism |
| 6561 | 5.83E−04 | 8.05E−03 | 2 | 5 | proline biosynthesis |
| 9737 | 5.92E−04 | 8.05E−03 | 8 | 241 | response to abscisic acid stimulus |
| 40007 | 6.59E−04 | 8.74E−03 | 8 | 245 | growth |
| 45859 | 7.14E−04 | 9.04E−03 | 3 | 23 | regulation of protein kinase activity |
| 43549 | 7.14E−04 | 9.04E−03 | 3 | 23 | regulation of kinase activity |
| 43285 | 7.46E−04 | 9.23E−03 | 9 | 312 | biopolymer catabolism |
| 7148 | 7.91E−04 | 9.46E−03 | 8 | 252 | cell morphogenesis |
| 51338 | 8.12E−04 | 9.46E−03 | 3 | 24 | regulation of transferase activity |
| 6092 | 8.18E−04 | 9.46E−03 | 5 | 94 | main pathways of carbohydrate metabolism |
| 16049 | 9.82E−04 | 1.11E−02 | 7 | 201 | cell growth |
| 43170 | 1.08E−03 | 1.20E−02 | 69 | 6392 | macromolecule metabolism |
| 8361 | 1.13E−03 | 1.23E−02 | 7 | 206 | regulation of cell size |
| 30163 | 1.17E−03 | 1.24E−02 | 8 | 268 | protein catabolism |
| 50789 | 1.18E−03 | 1.24E−02 | 32 | 2359 | regulation of biological process |
| 50791 | 1.26E−03 | 1.30E−02 | 29 | 2070 | regulation of physiological process |
| 6091 | 1.29E−03 | 1.30E−02 | 8 | 272 | generation of precursor metabolites and energy |
| 51244 | 1.34E−03 | 1.33E−02 | 28 | 1980 | regulation of cellular physiological process |
| 16043 | 1.47E−03 | 1.42E−02 | 22 | 1419 | cell organization and biogenesis |
| 6560 | 1.61E−03 | 1.42E−02 | 2 | 8 | proline metabolism |
| 6469 | 1.61E−03 | 1.42E−02 | 2 | 8 | negative regulation of protein kinase activity |
| 45736 | 1.61E−03 | 1.42E−02 | 2 | 8 | negative regulation of cyclin-dependent protein |
| | | | | | kinase activity |
| 51348 | 1.61E−03 | 1.42E−02 | 2 | 8 | negative regulation of transferase activity |
| 1558 | 1.61E−03 | 1.42E−02 | 2 | 8 | regulation of cell growth |
| 48856 | 1.64E−03 | 1.42E−02 | 20 | 1248 | anatomical structure development |
| 9725 | 1.65E−03 | 1.42E−02 | 14 | 729 | response to hormone stimulus |
| 44238 | 1.81E−03 | 1.53E−02 | 85 | 8429 | primary metabolism |
| 8152 | 1.82E−03 | 1.53E−02 | 98 | 10040 | metabolism |
| 9409 | 1.92E−03 | 1.59E−02 | 7 | 226 | response to cold |
| 45786 | 2.06E−03 | 1.64E−02 | 2 | 9 | negative regulation of progression through cell cycle |
| 51302 | 2.06E−03 | 1.64E−02 | 2 | 9 | regulation of cell division |
| 50794 | 2.08E−03 | 1.64E−02 | 28 | 2040 | regulation of cellular process |
| 51170 | 2.27E−03 | 1.76E−02 | 3 | 34 | nuclear import |
| 9790 | 2.47E−03 | 1.89E−02 | 11 | 520 | embryonic development |
| 43086 | 2.56E−03 | 1.93E−02 | 2 | 10 | negative regulation of enzyme activity |
| 6395 | 2.59E−03 | 1.93E−02 | 4 | 74 | RNA splicing |
| 6099 | 3.11E−03 | 2.26E−02 | 2 | 11 | tricarboxylic acid cycle |
| 46356 | 3.11E−03 | 2.26E−02 | 2 | 11 | acetyl-CoA catabolism |
| 6511 | 3.30E−03 | 2.30E−02 | 7 | 249 | ubiquitin-dependent protein catabolism |
| 43632 | 3.30E−03 | 2.30E−02 | 7 | 249 | modification-dependent macromolecule catabolism |
| 19941 | 3.30E−03 | 2.30E−02 | 7 | 249 | modification-dependent protein catabolism |
| 51603 | 3.45E−03 | 2.37E−02 | 7 | 251 | proteolysis during cellular protein catabolism |
| 6270 | 3.71E−03 | 2.53E−02 | 2 | 12 | DNA replication initiation |
| 9064 | 3.88E−03 | 2.55E−02 | 3 | 41 | glutamine family amino acid metabolism |
| 9615 | 3.88E−03 | 2.55E−02 | 3 | 41 | response to virus |
| 44257 | 3.92E−03 | 2.55E−02 | 7 | 257 | cellular protein catabolism |
| 48827 | 3.94E−03 | 2.55E−02 | 6 | 193 | phyllome development |
| 51169 | 4.16E−03 | 2.66E−02 | 3 | 42 | nuclear transport |
| 16071 | 4.40E−03 | 2.78E−02 | 5 | 138 | mRNA metabolism |
| 9793 | 4.72E−03 | 2.95E−02 | 10 | 487 | embryonic development (sensu Magnoliophyta) |
| 9109 | 5.07E−03 | 3.13E−02 | 2 | 14 | coenzyme catabolism |
| 6800 | 5.22E−03 | 3.19E−02 | 7 | 271 | oxygen and reactive oxygen species metabolism |
| 398 | 5.38E−03 | 3.25E−02 | 3 | 46 | nuclear mRNA splicing, via spliceosome |
| 48367 | 5.43E−03 | 3.25E−02 | 7 | 273 | shoot development |
| 6096 | 5.71E−03 | 3.38E−02 | 3 | 47 | glycolysis |
| 9060 | 5.82E−03 | 3.40E−02 | 2 | 15 | aerobic respiration |
| 375 | 6.06E−03 | 3.47E−02 | 3 | 48 | RNA splicing, via transesterification reactions |
| 377 | 6.06E−03 | 3.47E−02 | 3 | 48 | RNA splicing, via transesterification reactions with |
| | | | | | bulged adenosine as nucleophile |
| 9084 | 7.46E−03 | 4.23E−02 | 2 | 17 | glutamine family amino acid biosynthesis |
| 42891 | 7.71E−03 | 4.24E−02 | 1 | 1 | antibiotic transport |
| 7091 | 7.71E−03 | 4.24E−02 | 1 | 1 | mitotic metaphase/anaphase transition |
| 30071 | 7.71E−03 | 4.24E−02 | 1 | 1 | regulation of mitotic metaphase/anaphase transition |
| 48316 | 8.36E−03 | 4.55E−02 | 10 | 530 | seed development |
| 48608 | 8.68E−03 | 4.68E−02 | 10 | 533 | reproductive structure development |
| 19952 | 8.92E−03 | 4.76E−02 | 13 | 794 | reproduction |
| 6732 | 9.45E−03 | 4.99E−02 | 5 | 166 | coenzyme metabolism |

TABLE 4
Overview of cell cycle-related features used in the computational
analysis to search for new cell cycle proteins.

| Cell cycle feature | Category | # of genes | References |
|---|---|---|---|
| Periodicity | Gene expression | 1258 | (5, 6) |
| MSA-like | Promotor motif | 2295 | (7) |
| E2Fa-like | Promotor motif | 1809 | (7) |
| E2F10SPCNA | Promotor motif | 2221 | (7) |
| OS_motifslandIIa | Promotor motif | 2310 | (7) |
| UP1ATMSD | Promotor motif | 3738 | (7) |
| wrrmGCGn | Promotor motif | 2179 | (7) |
| CDK consensus site | Phosphorylation motif | 6321 | (8) |
| [IM]R-tail | Protein sequence motif | 116 | (16, 17) |
| PEST-sequence | Destruction motif | 2719 | (18) |
| D-box | Destruction motif | 2369 | (19) |
| KEN-box | Destruction motif | 410 | (19) |
| GxEN-box | Destruction motif | 300 | (20) |
| A-box | Destruction motif | 1779 | (21) |

TABLE 5
collection of cell cycle genes.
Collection of 518 genes annotated as cell cycle by gene ontology, including all genes
described as core cell cycle genes or related to cell cycle (19, 22-24). The following GO terms
were used to build the collection: cell cycle (GO: 0007049), cell division (GO: 0051301), cell
proliferation (GO: 0008283), chromosome segregation (GO: 0007059), DNA replication
(GO: 0006260), DNA replication and chromosome cycle (GO: 0000067). For every gene, the
number of cell cycle-related features present is shown. This collection was subtracted from the
prey lists to find novel cell cycle proteins.

| Accession | | |
|---|---|---|
| number | Name | # features |
| AT2G31270 | CDT1a | 8 |
| AT5G13840 | CCS52B | 7 |
| AT3G16320 | cdc27a | 7 |
| AT1G07270 | cdc6b | 7 |
| AT3G59550 | cohesion family protein SYN3 (SYN3) | 7 |
| AT5G25380 | CYCA2; 1 | 7 |
| AT3G11450 | DNAJ HEAT SHOCK N-TERMINAL DOMAIN-CONTAINING PROTEIN/ | 7 |
| | CELL DIVISION PROTEIN-RELATED | |
| AT1G65470 | chromatin assembly factor-1 (FASCIATA1) (FAS1), identical to FAS1 | 6 |
| | (Arabidopsis thaliana) GI: 4887626 | |
| AT1G77390 | CYCA1; 2 | 6 |
| AT5G43080 | CYCA3; 1 | 6 |
| AT3G11520 | CYCB1; 3 | 6 |
| AT3G10690 | DNA gyrase subunit A family protein | 6 |
| AT4G25540 | DNA mismatch repair protein MSH3; AtMsh3 | 6 |
| AT1G47870 | E2Fc | 6 |
| AT4G38600 | KAK (KAKTUS) | 6 |
| AT5G46280 | MCM3 | 6 |
| AT5G07280 | receptor-like protein kinase-like protein; receptor-like protein kinase - | 6 |
| | lpomoea nil (Japanese morning glory), PIR: T18536 | |
| AT5G61000 | Replication protein A1; Putative RNA helicase | 6 |
| AT5G42190 | ASK2 (ARABIDOPSIS SKP1-LIKE 2); UBIQUITIN-PROTEIN LIGASE | 5 |
| AT2G29680 | cdc6 | 5 |
| AT1G18670 | CKL3 | 5 |
| AT1G70210 | CYCD1; 1 | 5 |
| AT4G37630 | CYCD5; 1 | 5 |
| AT5G49160 | DNA (cytosine-5-)-methyltransferase (AtHIM), identical to SP: P34881 | 5 |
| | DNA (cytosine-5)-methyltransferase AthI (EC 2.1.1.37) {Arabidopsis | |
| | thaliana} | |
| AT1G08130 | DNA ligase/polydeoxyribonucleotide synthase [AtP] identical to | 5 |
| | SP\|Q42572 DNA ligase (EC 6.5.1.1) (Polydeoxyribonucleotide | |
| | synthase [AtP]) {Arabidopsis thaliana}; contains Pfam profiles: | |
| | PF01068 AtP dependent DNA ligase domain, PF04679 AtP dependent | |
| | DNA | |
| AT5G41880 | DNA polymerase alpha subunit IV (Primase)-like protein | 5 |
| AT3G13170 | DNA TOPOISOMERASE VIA (SPO11-1), IDENTICAL TO ATSPO11-1 | 5 |
| | (ARABIDOPSIS THALIANA) GI: 13383478; CONTAINS PFAM | |
| | PROFILE PF04406: TYPE IIB DNA TOPOISOMERASE; IDENTICAL | |
| | TO CDNA PUTATIVE TOPOISOMERASE VIA (SPO11 GENE 1) | |
| | GI: 7270974\|CHR3: 4231567-4234199 REVERSE\|ALIASES: | |
| | MJG19.19 | |
| AT5G67100 | DNA-directed DNA polymerase alpha catalytic subunit, putative, similar | 5 |
| | to SP: O48653 DNA polymerase alpha catalytic subunit (EC 2.7.7.7) | |
| | {Oryza sativa}; contains Pfam profiles: PF03175 DNA polymerase type | |
| | B, organellar and viral, PF00136 DNA polymerase fa | |
| AT5G02470 | DPa | 5 |
| AT5G22220 | E2Fb | 5 |
| AT3G43210 | kinesin motor family protein (NACK2) | 5 |
| AT1G18800 | NRP2 (NAP1-RELATED PROTEIN 2); DNA BINDING/CHROMATIN | 5 |
| | BINDING/HISTONE BINDING | |
| AT2G37560 | ORC2 | 5 |
| AT1G26840 | ORC6 | 5 |
| AT5G16270 | Rad21/Rec8-like family protein weak similarity to cohesion family | 5 |
| | protein SYN2 [Arabidopsis thaliana] gI: 12006360; contains Pfam | |
| | profiles PF04824: Conserved region of Rad21/Rec8 like protein, | |
| | PF04825: N terminus of Rad21/Rec8 like protein; supporting | |
| AT3G12280 | RBR | 5 |
| AT5G61210 | SNAP33 (SYNAPTOSOMAL-ASSOCIATED PROTEIN 33); T-SNARE | 5 |
| AT5G48600 | structural maintenance of chromosomes (SMC) family protein, similar | 5 |
| | to SP: P50532 Chromosome assembly protein XCAP-C {Xenopus | |
| | laevis}; contains Pfam profiles PF02483: SMC family C-terminal | |
| | domain, PF02463: RecF/RecN/SMC N terminal domain | |
| AT5G55220 | trigger factor type chaperone family protein, contains Pfam profiles | 5 |
| | PF05697: Bacterial trigger factor protein (TF), PF05698: Bacterial | |
| | trigger factor protein (TF) C-terminus, PF00254: peptidyl-prolyl | |
| | cis-trans isomerase, FKBP-type | |
| AT3G60740 | tubulin folding cofactor D | 5 |
| AT3G19770 | vacuolar sorting protein 9 domain-containing protein | 5 |
| AT5G05560 | APC1 | 4 |
| AT1G06590 | APC5 | 4 |
| AT3G28730 | ATHMG (HIGH MOBILITY GROUP, STRUCTURE-SPECIFIC | 4 |
| | RECOGNITION PROTEIN 1); TRANSCRIPTION FACTOR | |
| AT3G53230 | CDC48, putative | 4 |
| AT3G01610 | CDC48C\|AAA-TYPE ATPASE FAMILY PROTEIN, CONTAINS PFAM | 4 |
| | DOMAIN, PF00004: ATPASE, AAA FAMILY (‘A’TPASES | |
| | ‘A’SSOCIATED WITH DIVERSE CELLULAR ‘A’CTIVITIES)\| | |
| | CHR3: 231658-235196 FORWARD\|ALIASES: EMB1354, EMBRYO | |
| | DEFECTIVE 1354, F4P135, F4P13_15 | |
| AT1G18040 | CDKD; 3 | 4 |
| AT5G03340 | CELL DIVISION CONTROL PROTEIN 48 HOMOLOG E; ATPASE | 4 |
| AT5G11300 | CYCA2; 2 | 4 |
| AT1G15570 | CYCA2; 3 | 4 |
| AT1G80370 | CYCA2; 4 | 4 |
| AT2G26760 | CYCB1; 4 | 4 |
| AT2G17620 | CYCB2; 1 | 4 |
| AT4G35620 | CYCB2; 2 | 4 |
| AT3G50070 | CYCD3; 3 | 4 |
| AT3G05330 | cyclin family low similarity to microtubule-binding protein TANGLED1 | 4 |
| AT5G05490 | DIF1/SYN1 | 4 |
| AT1G69770 | DNA (cytosine-5)-methyltransferase CMt3; Chromomethylase 3; | 4 |
| | Protein CHROMOMETHYLASE3 | |
| AT4G02460 | DNA mismatch repair protein | 4 |
| AT1G67320 | DNA primase, large subunit family, contains Pfam profile PF04104: | 4 |
| | Eukaryotic-type DNA primase, large subunit; similar to DNA primase | |
| | large subunit (EC 2.7.7.—) (DNA primase 58 kDa subunit) (p58) | |
| | (Swiss-Prot: P49643) (Homo sapiens) | |
| AT4G31210 | DNA topoisomerase family protein, similar to DNA Topoisomerase I | 4 |
| | (SP: Q9X3X7) {Zymomonas mobilis} | |
| AT5G63920 | DNA topoisomerase III | 4 |
| AT5G06110 | DNAJ heat shock N-terminal domain-containing protein/cell division | 4 |
| | protein-related, similar to GlsA (Volvox carteri f. nagariensis) | |
| | GI: 4633129; contains Pfam profiles PF00226 DnaJ domain, PF00249 | |
| | Myb-like DNA-binding domain | |
| AT1G78650 | expressed protein, weak similarity to DNA polymerase delta subunit 3 | 4 |
| | (DNA polymerase delta subunit p66) (Swiss-Prot: Q15054) (Homo | |
| | sapiens) | |
| AT2G13330 | F14O4.9 | 4 |
| AT2G23430 | KRP1 | 4 |
| AT2G20980 | MCM10 | 4 |
| AT2G29570 | PCNA2 | 4 |
| AT4G14150 | phragmoplast-associated kinesin-related protein (PAKRP1) | 4 |
| AT3G23670 | phragmoplast-associated kinesin-related protein, putative, similar to | 4 |
| | kinesin-like protein GB: CAB10194 from (Arabidopsis thaliana) | |
| AT5G58720 | PRLI-INTERACTING FACTOR, PUTATIVE | 4 |
| AT1G53710 | PROTEIN SERINE/THREONINE PHOSPHATASE | 4 |
| AT3G02920 | replication protein-related, similar to replication protein A 30 kDa (Oryza | 4 |
| | sativa (japonica cultivar-group)) GI: 13516746; contains InterPro entry | |
| | IPR004365: OB-fold nucleic acid binding domain | |
| AT4G22970 | similar to hypothetical protein [Arabidopsis thaliana] | 4 |
| | (TAIR: At5g28550.1); similar to BIMB_EMENI CELL | |
| | DIVISION-ASSOCIATED PROTEIN BIMB [Aspergillus nidulans FGSC | |
| | A4] (GB: EAA60576.1); similar to probable spindle pole body | |
| | component bimB - Emericella nidula | |
| AT5G61460 | SMC-like protein | 4 |
| AT1G65660 | SMP1 (SWELLMAP 1); NUCLEIC ACID BINDING | 4 |
| AT5G62000 | transcriptional factor B3 family protein/auxin-responsive factor | 4 |
| AT5G16750 | transducin family protein/WD-40 repeat family protein contains 8 | 4 |
| | WD-40 repeats (PF00400); similar to transducin homolog sazD - Homo | |
| | sapiens, EMBL: U02609 | |
| AT2G14400 | transposable element gene | 4 |
| AT2G42260 | UVI4 | 4 |
| AT1G57820 | VARIANT IN METHYLATION 1 | 4 |
| AT3G02820 | zinc knuckle (CCHC-type) family protein, contains Pfam domain, | 4 |
| | PF00098: Zinc knuckle | |
| AT5G22010 | AAA-TYPE ATPASE FAMILY PROTEIN/BRCT | 3 |
| | DOMAIN-CONTAINING PROTEIN | |
| AT2G14120 | ADL2b | 3 |
| AT5G57160 | ATLIG4 (ARABIDOPSIS THALIANA DNA LIGASE IV) | 3 |
| AT5G45720 | ATP BINDING/DNA-DIRECTED DNA POLYMERASE/ | 3 |
| | NUCLEOSIDE-TRIPHOSPHATASE/NUCLEOTIDE BINDING | |
| AT1G66730 | ATP DEPENDENT DNA LIGASE FAMILY PROTEIN | 3 |
| AT2G32590 | barren family protein, low similarity to SP: Q9Y7R3 Condensin complex | 3 |
| | subunit 2 (p105) {Schizosaccharomyces pombe}; contains Pfam profile | |
| | PF05786: Barren protein | |
| AT3G19590 | BUB3-like2 | 3 |
| AT4G22910 | CCS52A1 | 3 |
| AT4G11920 | CCS52A2 | 3 |
| AT4G33270 | cdc20-1 | 3 |
| AT3G25100 | CDC45 (Cell division cycle 45)-like protein; Putative cell division cycle | 3 |
| | protein 45 | |
| AT3G09840 | CDC48 | 3 |
| AT1G20930 | CDKB2; 2 | 3 |
| AT1G73690 | CDKD; 1 | 3 |
| AT4G28980 | CDKF; 1 | 3 |
| AT2G32900 | centromere/kinetochore protein, putative (ZW10) | 3 |
| AT5G62410 | Chromosome assembly protein homolog | 3 |
| AT1G44110 | CYCA1; 1 | 3 |
| AT1G47210 | CYCA3; 2 | 3 |
| AT4G37490 | CYCB1; 1 | 3 |
| AT1G76310 | CYCB2; 4 | 3 |
| AT4G34160 | CYCD3; 1 | 3 |
| AT4G34090 | CYL1 | 3 |
| AT3G20550 | DDL (DAWDLE) | 3 |
| AT5G14960 | DEL2 | 3 |
| AT4G09140 | DNA mismatch repair protein MLH1 | 3 |
| AT3G18524 | DNA mismatch repair protein MSH2 (MSH2) identical to SP\|O24617 | 3 |
| | DNA mismatch repair protein MSH2 (AtMsh2) {Arabidopsis thaliana} | |
| AT4G02070 | DNA mismatch repair protein MSH6-1 (MSH6-1) (AGAA.3) | 3 |
| AT5G55300 | DNA topoisomerase I | 3 |
| AT3G17830 | DNAJ HEAT SHOCK FAMILY PROTEIN | 3 |
| AT1G80030 | DNAJ HEAT SHOCK PROTEIN, PUTATIVE | 3 |
| AT1G08840 | EMB2411\|DNA REPLICATION HELICASE, PUTATIVE, SIMILAR TO | 3 |
| | HELICASE (XENOPUS LAEVIS) GI: 18845092: GB: AAL79550\| | |
| | CHR1: 2829582-2838372 REVERSE\|ALIASES: EMB2411, EMBRYO | |
| | DEFECTIVE 2411, F7G19.26 | |
| AT4G37120 | expressed protein | 3 |
| AT5G58870 | FTSH9 (FTSH PROTEASE 9); ATP-DEPENDENT PEPTIDASE/ | 3 |
| | ATPASE/METALLOPEPTIDASE | |
| AT1G13980 | GN (GNOM) | 3 |
| AT5G24330 | Histone-lysine N-methyltransferase AtXR6; Protein SET DOMAIN | 3 |
| | GROUP 34; trithorax-related protein 6; tRX-related protein 6 | |
| AT5G06830 | IDENTICAL TO CDK5RAP3-LIKE PROTEIN [ARABIDOPSIS | 3 |
| | THALIANA] (GB: Q9FG23); SIMILAR TO OS06G0320100 [ORYZA | |
| | SATIVA (JAPONICA CULTIVAR-GROUP)] (GB: NP_001057510.1); | |
| | SIMILAR TO CDK5 REGULATORY SUBUNIT ASSOCIATED | |
| | PROTEIN 3 [MUS MUSCULUS] (GB: NP_084524.1); CONTAINS | |
| | INTERPRO DOMAIN PROTEIN OF UNKNOWN FUNCTION DUF773; | |
| | (INTERPRO: IPR008491) | |
| AT1G49620 | KRP7 | 3 |
| AT1G71830 | leucine-rich repeat family protein/protein kinase family protein, | 3 |
| | contains Pfam domains PF00560: Leucine Rich Repeat and PF00069: | |
| | Protein kinase domain | |
| AT1G80080 | leucine-rich repeat family protein, contains leucine rich-repeat domains | 3 |
| | Pfam: PF00560, INTERPRO: IPR001611; contains some similarity to | |
| | Hcr2-5D (Lycopersicon esculentum) gi: 3894393: gb: AAC78596 | |
| AT3G25980 | MAD2-like | 3 |
| AT5G51600 | MAP65-3 | 3 |
| AT1G44900 | MCM2 | 3 |
| AT2G16440 | MCM4 | 3 |
| AT2G07690 | MCM5 | 3 |
| AT5G54260 | MRE11\|DNA REPAIR AND MEIOSIS PROTEIN (MRE11), | 3 |
| | IDENTICAL TO DNA REPAIR AND MEIOSIS PROTEIN (MRE11) | |
| | GI: 5524769 FROM (ARABIDOPSIS THALIANA)\| | |
| | CHR5: 22049295-22055217 FORWARD\|ALIASES: MDK4.8, MDK4_8 | |
| AT5G58230 | MSI1 (MULTICOPY SUPRESSOR OF IRA1) | 3 |
| AT1G61000 | Nuf2 family protein contains Pfam PF03800: Nuf2 family | 3 |
| | domain/kinetochore protein NUF2 | |
| AT5G16690 | ORC3 | 3 |
| AT1G72150 | PATL1 (PATELLIN 1); TRANSPORTER | 3 |
| AT1G50840 | poll-like DNA polymerase, putative, similar to Poll-like DNA | 3 |
| | polymerase (Oryza sativa) GI: 19912795 contains Pfam profiles | |
| | PF01612: 3′-5′ exonuclease, PF00476: DNA polymerase I family A | |
| AT1G80190 | PSF1 (GINS complex DNA replication) | 3 |
| AT2G45280 | putative RAD51C-like DNA repair protein; supported by cDNA: | 3 |
| | gi_15425730_dbj_AB062456.1_AB062456 | |
| AT2G31970 | RAD50\|DNA REPAIR-RECOMBINATION PROTEIN (RAD50), | 3 |
| | IDENTICAL TO DNA REPAIR-RECOMBINATION PROTEIN | |
| | GI: 7110148 FROM (ARABIDOPSIS THALIANA)\| | |
| | CHR2: 13607589-13616146 FORWARD\|ALIASES: ATRAD50, DNA | |
| | REPAIR RECOMBINATION PROTEIN, F22D22.28, F22D22_28 | |
| AT5G08020 | Replication factor A-like protein | 3 |
| AT2G21790 | Ribonucleoside-diphosphate reductase large subunit; AtRNR1; | 3 |
| | Ribonucleoside-diphosphate reductase R1 subunit | |
| AT3G27060 | ribonucleoside-diphosphate reductase small chain, putative/ | 3 |
| | ribonucleotide reductase, putative, similar to ribonucleotide reductase | |
| | R2 (Nicotiana tabacum) GI: 1044912; contains Pfam profile PF00268: | |
| | Ribonucleotide reductase, small chain | |
| AT2G24490 | RPA2 | 3 |
| AT4G18590 | RPA3 | 3 |
| AT1G30690 | SEC14 cytosolic factor family protein/phosphoglyceride transfer family | 3 |
| | protein, contains Pfam PF00650: CRAL/TRIO domain; contains Pfam | |
| | PF03765: CRAL/TRIO, N-terminus; similar to cytosolic factor | |
| | (Phosphatidylinositol/phosphatidylcholine transfer prot | |
| AT1G74560 | similar to nucleosome assembly protein (NAP) family protein | 3 |
| | [Arabidopsis thaliana] (TAIR: At1g18800.1); similar to | |
| | nucleosome/chromatin assembly factor A [Zea mays] | |
| | (GB: AAK67146.1); contains InterPro domain Nucleosome assembly | |
| | protein (NAP) (InterPro: IPR00 | |
| AT5G04320 | similar to unknown protein [Arabidopsis thaliana] (TAIR: AT3G10440.1); | 3 |
| | similar to hypothetical protein Osl_009118 [Oryza sativa (indica | |
| | cultivar-group)] (GB: EAY87885.1); contains InterPro domain | |
| | Shugoshin, C-terminal; (InterPro: IPR011515) | |
| AT5G49010 | SLD5 (DNA replication) | 3 |
| AT5G51330 | SWI1 (SWITCH1); PHOSPHOLIPASE C | 3 |
| AT4G02110 | TOPBP1 | 3 |
| AT2G01550 | transposable element gene | 3 |
| AT2G15410 | transposable element gene | 3 |
| AT4G33790 | ACYL COA REDUCTASE, PUTATIVE | 2 |
| AT5G22420 | ACYL COA REDUCTASE, PUTATIVE | 2 |
| AT4G16420 | ADA2B (PROPORZ1); DNA BINDING/TRANSCRIPTION FACTOR | 2 |
| AT1G78580 | alpha, alpha-trehalose-phosphate synthase, UDP-forming, putative | 2 |
| | (Fragment) | |
| AT1G54960 | ANP2 (ARABIDOPSIS NPK1-RELATED PROTEIN KINASE 2); | 2 |
| | KINASE | |
| AT4G37750 | ANT | 2 |
| AT1G78770 | APC6 | 2 |
| AT2G39090 | APC7 | 2 |
| AT3G48150 | APC8 | 2 |
| AT4G30080 | ARF16 (AUXIN RESPONSE FACTOR 16); MIRNA BINDING/ | 2 |
| | TRANSCRIPTION FACTOR | |
| AT1G50240 | armadillo/beta-catenin repeat family protein, contains Pfam profile: | 2 |
| | PF00514 armadillo/beta-catenin-like repeat | |
| AT1G67370 | ASY1 (ASYNAPTIC 1); DNA BINDING | 2 |
| AT3G12400 | ATELC/ELC; ubiquitin binding | 2 |
| AT3G52115 | ATGR1 (GAMMA RESPONSE 1) | 2 |
| AT5G55230 | ATMAP65-1 (MICROTUBULE-ASSOCIATED PROTEINS 65-1); | 2 |
| | MICROTUBULE BINDING | |
| AT3G27730 | ATP-dependent DNA helicase, putative | 2 |
| AT5G40820 | ATR | 2 |
| AT5G16850 | ATTERT (TELOMERASE REVERSE TRANSCRIPTASE); | 2 |
| | TELOMERIC TEMPLATE RNA REVERSE TRANSCRIPTASE | |
| AT4G00020 | BRCA2A (BREAST CANCER 2 LIKE 2A, EMBRYO SAC | 2 |
| | DEVELOPMENT ARREST 20, MATERNAL EFFECT EMBRYO | |
| | ARREST 43) | |
| AT5G01630 | BRCA2B_ATBRCA2(V)_BRCA2(V)_BRCA2B (breast cancer 2 like | 2 |
| | 2B) | |
| AT2G13680 | CALS5 (CALLOSE SYNTHASE 5); 1,3-BETA-GLUCAN SYNTHASE | 2 |
| AT2G20000 | cdc27b | 2 |
| AT2G03670 | CDC48B; ATPASE | 2 |
| AT1G76540 | CDKB2; 1 | 2 |
| AT5G64960 | CDKC; 2 | 2 |
| AT5G63370 | CDKG; 1 | 2 |
| AT3G02450 | CELL DIVISION PROTEIN FTSH, PUTATIVE | 2 |
| AT5G55280 | cell division protein FtsZ, chloroplast, putative | 2 |
| AT5G37630 | chromosome condensation family protein, contains pfam profile: | 2 |
| | PF04154 chromosome condensation protein 3, C-terminal region | |
| AT1G09600 | CKL11 | 2 |
| AT4G22940 | CKL4 | 2 |
| AT5G44290 | CKL5 | 2 |
| AT1G03740 | CKL6 | 2 |
| AT1G54610 | CKL9 | 2 |
| AT2G27970 | CKS2 | 2 |
| AT5G40840 | cohesion family protein SYN2 (SYN2) | 2 |
| AT4G02570 | cullin family protein NCBI Chain C, Crystal Structure Of the | 2 |
| | Ddb1-Cul4a-Rbx1-Sv5v Complex | |
| AT1G26830 | cullin3a | 2 |
| AT5G06150 | CYCB1; 2 | 2 |
| AT1G34460 | CYCB1; 5 | 2 |
| AT5G48640 | CYCC1; 1 | 2 |
| AT2G26430 | CYCL1 | 2 |
| AT4G19560 | CYCT1; 2 | 2 |
| AT5G45190 | CYCT1; 5 | 2 |
| AT3G01330 | DEL3 | 2 |
| AT4G35520 | DNA mismatch repair family protein | 2 |
| AT1G65070 | DNA MISMATCH REPAIR MUTS FAMILY PROTEIN | 2 |
| AT1G67630 | DNA polymerase alpha subunit B family, contains Pfam profile: | 2 |
| | PF04058 DNA polymerase alpha subunit B | |
| AT1G09815 | DNA polymerase delta subunit 4 family, contains similarity to | 2 |
| | Swiss-Prot: Q9HCU8 DNA polymerase delta subunit 4 (DNA | |
| | polymerase delta subunit p12) (Homo sapiens) | |
| AT5G64420 | DNA polymerase V family, contains Pfam domain PF04931: DNA | 2 |
| | polymerase V | |
| AT1G14460 | DNA polymerase-related, weak similarity to DNA polymerase III | 2 |
| | holoenzyme tau subunit (Thermus thermophilus) GI: 2583049 | |
| AT5G55310 | DNA topoisomerase I, putative | 2 |
| AT5G44610 | DREPP PLASMA MEMBRANE POLYPEPTIDE-RELATED | 2 |
| AT2G36010 | E2Fa | 2 |
| AT3G11220 | ELP4 | 2 |
| AT4G32700 | Encodes a homolog of Drosophila MUS308 and mammalian DNA | 2 |
| | polymerase, which prevent spontaneous or DNA damage-induced | |
| | production of DNA double strand breaks. | |
| AT5G64630 | Encodes the second largest subunit of the chromatin assembly | 2 |
| | factor-1. Involved in organization of the shoot and root apical | |
| | meristems. | |
| AT4G24790 | expressed protein; expression supported by MPSS | 2 |
| AT3G24495 | Expressed protein; GC donor splice site at exon 11; supported by | 2 |
| | cDNA: gi_6224916_gb_AF193018.1_AF193018 | |
| AT4G23940 | FTSH PROTEASE, PUTATIVE | 2 |
| AT5G01230 | FtsJ-like methyltransferase family protein, contains Pfam profile: | 2 |
| | PF01728 FtsJ-like methyltransferase | |
| AT1G48270 | GCR1 (G-PROTEIN-COUPLED RECEPTOR 1) | 2 |
| AT1G10270 | GRP23 (GLUTAMINE-RICH PROTEIN23); BINDING | 2 |
| AT2G26890 | GRV2 (KATAMARI2); binding/heat shock protein binding | 2 |
| AT4G32880 | homeobox-leucine zipper transcription factor (HB-8), identical to | 2 |
| | HD-zip transcription factor (athb-8) (GI: 7270235) (Arabidopsis thaliana) | |
| AT5G46920 | intron maturase, type II family protein | 2 |
| AT5G54670 | kinesin-like protein C (KATC) | 2 |
| AT3G19150 | KRP6 | 2 |
| AT5G44635 | MCM6 | 2 |
| AT4G02060 | MCM7 | 2 |
| AT3G09660 | MCM8 | 2 |
| AT2G14050 | MCM9 | 2 |
| AT1G77320 | MEI1 (MEIOSIS DEFECTIVE 1); TRANSCRIPTION COACTIVATOR | 2 |
| AT3G22880 | meiotic recombination protein, putative | 2 |
| AT5G50110 | METHYLTRANSFERASE-RELATED | 2 |
| AT5G24020 | MIND (ACCUMULATION AND REPLICATION OF CHLOROPLAST | 2 |
| | 11) | |
| AT3G24320 | mismatch binding protein, putative; similar to mismatch binding protein | 2 |
| | Mus3 GB: AAF35250 from (Zea mays) | |
| AT5G49880 | MITOTIC CHECKPOINT FAMILY PROTEIN | 2 |
| AT2G35630 | MOR1\|MICROTUBULE ORGANIZATION 1 PROTEIN (MOR1), | 2 |
| | IDENTICAL TO MICROTUBULE ORGANIZATION 1 PROTEIN | |
| | GI: 14317953 FROM (ARABIDOPSIS THALIANA)\| | |
| | CHR2: 14973757-14987661 FORWARD\|ALIASES: GEM1, | |
| | MICROTUBULE ORGANIZATION 1, T20F21.17, T20F21_17 | |
| AT4G17380 | MSH4, similar to DNA mismatch repair protein MSH2 | 2 |
| AT1G63680 | Mur ligase family protein, contains Pfam profile: PF01225 Mur ligase | 2 |
| | family, catalytic domain | |
| AT5G11510 | MYB 3R4 | 2 |
| AT3G02680 | NBS1 (NIJMEGEN BREAKAGE SYNDROME 1) | 2 |
| AT3G57060 | non-SMC condensin subunit, XCAP-D2/Cnd1 family protein, similar to | 2 |
| | 13S condensin XCAP-D2 subunit (Xenopus laevis) GI: 3764087; | |
| | contains Pfam profile PF04594: Non-SMC condensin subunit, | |
| | XCAP-D2/Cnd1 | |
| AT4G14700 | ORC1A | 2 |
| AT4G29910 | ORC5 | 2 |
| AT4G30825 | pentatricopeptide (PPR) repeat-containing protein, contains Pfam | 2 |
| | profile PF01535: PPR repeat | |
| AT5G14520 | pescadillo-related | 2 |
| AT2G13840 | PHP DOMAIN-CONTAINING PROTEIN | 2 |
| AT3G63120 | PLP1; 1 | 2 |
| AT1G50230 | protein kinase family protein, contains protein kinase domain, | 2 |
| | Pfam: PF00069 | |
| AT3G63130 | RAN GTPase activating protein 1 (RanGAP1), contains Pfam | 2 |
| | PF00560: Leucine Rich Repeat domains; identical to RAN GTPase | |
| | activating protein 1 (GI: 6708466)(Arabidopsis thaliana) | |
| AT1G16590 | REV7 (REVERSIONLESS 7); DNA BINDING | 2 |
| AT5G27740 | RFC3 | 2 |
| AT1G21690 | RFC4 | 2 |
| AT1G77470 | RFC5 | 2 |
| AT1G48380 | RHL1 (ROOT HAIRLESS 1) | 2 |
| AT4G33495 | RPD1 (ROOT PRIMORDIUM DEFECTIVE 1) | 2 |
| AT5G35770 | SAP (STERILE APETALA); TRANSCRIPTION FACTOR | 2 |
| AT1G72160 | SEC14 CYTOSOLIC FACTOR FAMILY PROTEIN/ | 2 |
| | PHOSPHOGLYCERIDE TRANSFER FAMILY PROTEIN | |
| AT4G09160 | SEC14 CYTOSOLIC FACTOR FAMILY PROTEIN/ | 2 |
| | PHOSPHOGLYCERIDE TRANSFER FAMILY PROTEIN | |
| AT3G51670 | SEC14 cytosolic factor family protein/phosphoglyceride transfer family | 2 |
| | protein, similar to polyphosphoinositide binding protein Ssh2p | |
| | (GI: 2739046) {Glycine max};; contains Pfam PF00650: CRAL/TRIO | |
| | domain; contains Pfam PF03765: CRAL/TRIO, N-terminus | |
| AT5G03730 | SERINE/THREONINE-PROTEIN KINASE CTR1 | 2 |
| AT4G14180 | SIMILAR TO OS04G0347800 [ORYZA SATIVA (JAPONICA | 2 |
| | CULTIVAR-GROUP)] (GB: NP_001052517.1); SIMILAR TO | |
| | OSIGBA0130K07.4 [ORYZA SATIVA (INDICA CULTIVAR-GROUP)] | |
| | (GB: CAH66368.1); SIMILAR TO OSJNBA0020I02.9 [ORYZA SATIVA | |
| | (JAPONICA CULTIVAR-GROUP)] (GB: CAE02100.2); CONTAINS | |
| | DOMAIN ARM REPEAT (SSF48371) | |
| AT1G08260 | SIMILAR TO POL2A. DNA POLYMERASE EPSILON CATALYTIC | 2 |
| | SUBUNIT. ESSENTIAL FOR ARABIDOPSIS GROWTH. NULL | |
| | HOMOZYGOTES ARE EMBRYO LETHAL. PARTIAL LOSS OF | |
| | FUNCTION ALLELES SHOW EMBRYO PATTERNING DEFECTS | |
| | SUCH AS ROOT POLE DISPLACEMENT. DELAYED | |
| | PROGRESSION THROUGH CELL CYCLE RESULTS IN EMBRYOS | |
| | WITH SMALLER NUMBERS OF LARGER CELLS. | |
| AT2G27170 | similar to SMC2-like condensin, putative (SMC2) (TITAN3) | 2 |
| AT5G24630 | SIMILAR TO UNKNOWN PROTEIN [ARABIDOPSIS THALIANA] | 2 |
| | (TAIR: AT2G22795.1); SIMILAR TO UNKNOWN PROTEIN [ORYZA | |
| | SATIVA (JAPONICA CULTIVAR-GROUP)] (GB: BAD13052.1); | |
| | SIMILAR TO OS02G0147700 [ORYZA SATIVA (JAPONICA | |
| | CULTIVAR-GROUP)] (GB: NP_001045886.1) | |
| AT3G10440 | SIMILAR TO UNKNOWN PROTEIN [ARABIDOPSIS THALIANA] | 2 |
| | (TAIR: AT5G04320 | |
| AT3G17590 | SNF5 homolog BSH (bsh) mRNA, complete cds | 2 |
| AT1G49040 | stomatal cytokinesis defective/SCD1 protein (SCD1) | 2 |
| AT5G15920 | structural maintenance of chromosomes (SMC) family protein (MSS2), | 2 |
| | similar to SMC-related protein MSS2 (Arabidopsis thaliana) | |
| | GI: 9965743 | |
| AT1G04110 | subtilase family protein, contains similarity to subtilisin-like protease | 2 |
| | gI: 3687307 | |
| AT3G04740 | SWP (STRUWWELPETER) | 2 |
| AT1G08560 | syntaxin-related protein KNOLLE | 2 |
| AT3G62980 | TIR1 (TRANSPORT INHIBITOR RESPONSE 1); ubiquitin-protein | 2 |
| | ligase | |
| AT1G30660 | toprim domain-containing protein | 2 |
| AT1G30680 | TOPRIM DOMAIN-CONTAINING PROTEIN | 2 |
| AT4G10710 | transcriptional regulator-related | 2 |
| AT3G44540 | TRANSDUCIN FAMILY PROTEIN/WD-40 REPEAT FAMILY | 2 |
| | PROTEIN, CONTAINS 6 (4 SIGNIFICANT) WD-40 REPEATS | |
| | (PF0400 | |
| AT2G15380 | transposable element gene | 2 |
| AT2G15540 | transposable element gene | 2 |
| AT2G18820 | transposable element gene | 2 |
| AT2G28980 | transposable element gene | 2 |
| AT4G04000 | transposable element gene | 2 |
| AT3G54670 | TTN8\|SIMILAR TO STRUCTURAL MAINTENANCE OF | 2 |
| | CHROMOSOMES (SMC) FAMILY PROTEIN [ARABIDOPSIS | |
| | THALIANA] (TAIR: AT5G48600.1 | |
| AT3G20060 | UBC19 (UBIQUITIN-CONJUGATING ENZYME 19); UBIQUITIN- | 2 |
| | PROTEIN LIGASE | |
| AT1G50490 | UBC20 | 2 |
| AT1G02970 | WEE1 | 2 |
| AT5G08290 | YLS8 (yellow-leaf-specific gene 8); catalytic | 2 |
| AT4G02980 | ABP1 (ENDOPLASMIC RETICULUM AUXIN BINDING PROTEIN 1) | 1 |
| AT5G22500 | ACYL COA REDUCTASE, PUTATIVE/MALE-STERILITY PROTEIN, | 1 |
| | PUTATIVE | |
| AT1G14830 | ADL1C/ADL5 (DYNAMIN-LIKE PROTEIN 5); GTP binding/GTPase | 1 |
| AT4G33650 | ADL2a | 1 |
| AT5G61960 | AML1 (ARABIDOPSIS MEI2-LIKE PROTEIN 1); RNA binding | 1 |
| AT4G01540 | ANAC068/NTM1 (NAC WITH TRANSMEMBRANE MOTIF1); | 1 |
| | TRANSCRIPTION FACTOR/TRANSCRIPTIONAL ACTIVATOR | |
| AT2G18290 | APC10 | 1 |
| AT2G04660 | APC2 | 1 |
| AT4G21530 | APC4 | 1 |
| AT3G54710 | ATCDT1B/CDT1/CDT1B (ARABIDOPSIS HOMOLOG OF YEAST | 1 |
| | CDT1 B); CYCLIN-DEPENDENT PROTEIN KINASE | |
| AT2G20190 | ATCLASP/CLASP; binding | |
| AT4G18820 | ATP BINDING/DNA-DIRECTED DNA POLYMERASE/ | 1 |
| | NUCLEOSIDE-TRIPHOSPHATASE/NUCLEOTIDE BINDING | |
| AT4G21800 | ATP-binding family protein | 1 |
| AT3G49780 | ATPSK3\|PHYTOSULFOKINES 3 (PSK3), IDENTICAL TO | 1 |
| | PHYTOSULFOKINES 3 (PSK3) SP: Q9M2Y0 FROM (ARABIDOPSIS | |
| | THALIANA)\|CHR3: 18476664-18477361 FORWARD\|ALIASES: | |
| | PHYTOSULFOKINE 3 PRECURSOR, T16K530 | |
| AT1G58470 | ATRBP1 (ARABIDOPSIS THALIANA RNA-BINDING PROTEIN 1); | 1 |
| | RNA binding | |
| AT4G00100 | ATRPS13A (RIBOSOMAL PROTEIN S13A); structural constituent of | 1 |
| | ribosome | |
| AT3G20780 | ATTOP6B (BRASSINOSTEROID INSENSITIVE 3, ROOT HAIRLESS | 1 |
| | 3) | |
| AT2G28350 | auxin-responsive factor (ARF10) | 1 |
| AT1G69400 | BUB3-like1 | 1 |
| AT5G27080 | cdc20-3 | 1 |
| AT5G03455 | cdc25 | 1 |
| AT2G38620 | CDKB1; 2 | 1 |
| AT5G10270 | CDKC; 1 | 1 |
| AT1G66750 | CDKD; 2 | 1 |
| AT5G63610 | CDKE; 1 | 1 |
| AT1G67580 | CDKG; 2 | 1 |
| AT2G46520 | CELLULAR APOPTOSIS SUSCEPTIBILITY PROTEIN, PUTATIVE/ | 1 |
| | IMPORTIN-ALPHA RE-EXPORTER, PUTATIVE | |
| AT2G25170 | chromatin remodeling factor CHD3 (PICKLE) | 1 |
| AT2G26280 | CID7; ATP BINDING/DAMAGED DNA BINDING | 1 |
| AT5G39420 | CKL1 | 1 |
| AT1G57700 | CKL10 | 1 |
| AT1G71530 | CKL12 | 1 |
| AT4G10010 | CKL13 | 1 |
| AT1G33770 | CKL14 | 1 |
| AT1G74330 | CKL2 | 1 |
| AT5G50860 | CKL7 | 1 |
| AT2G27960 | CKS1 | 1 |
| AT1G02980 | cullin family protein, similar to cullin 1 (Homo sapiens) GI: 3139077; | 1 |
| | contains Pfam profile PF00888: Cullin family | |
| AT1G69670 | cullin3b | 1 |
| AT5G46210 | cullin4 | 1 |
| AT1G47220 | CYCA3; 3 | 1 |
| AT1G47230 | CYCA3; 4 | 1 |
| AT1G20590 | CYCB2; 5 | 1 |
| AT1G16330 | CYCB3; 1 | 1 |
| AT2G22490 | CYCD2; 1 | 1 |
| AT5G67260 | CYCD3; 2 | 1 |
| AT5G65420 | CYCD4; 1 | 1 |
| AT4G03270 | CYCD6; 1 | 1 |
| AT5G27620 | CYCH; 1 | 1 |
| AT3G21870 | CYCLIN FAMILY PROTEIN, SIMILAR TO CYCLIN 2 | 1 |
| | (TRYPANOSOMA BRUCEI) GI: 7339572, CYCLIN 6 (TRYPANOSOMA | |
| | CRUZI) GI: 12005317; CONTAINS PFAM PROFILE PF00134: | |
| | CYCLIN, N-TERMINAL DOMAIN\|CHR3: 7703878-7704836 | |
| | REVERSE\|ALIASES: MEK6.1 | |
| AT5G50375 | cyclopropyl isomerase (CPI1) | 1 |
| AT2G45080 | CYCP3; 1 (CYCLIN P3; 1); CYCLIN-DEPENDENT PROTEIN KINASE | 1 |
| AT3G60550 | CYCP3; 2 | 1 |
| AT5G61650 | CYCP4; 2\|CYCLIN FAMILY PROTEIN, SIMILAR TO CYCLIN 2 | 1 |
| | (TRYPANOSOMA BRUCEI) GI: 7339572, CYCLIN 6 (TRYPANOSOMA | |
| | CRUZI) GI: 12005317; CONTAINS PFAM PROFILE PF00134: | |
| | CYCLIN, N-TERMINAL DOMAIN\|CHR5: 24795491-24796245 | |
| | FORWARD\|ALIASES: CYCP4, K11J9.17, K11J9_17 | |
| AT1G35440 | CYCT1; 1 | 1 |
| AT1G27630 | CYCT1; 3 | 1 |
| AT4G19600 | CYCT1; 4 | 1 |
| AT1G01040 | DEAD/DEAH box helicase carpel factory/CAF | 1 |
| AT3G48160 | DEL1 | 1 |
| AT5G54090 | DNA mismatch repair MutS family protein | 1 |
| AT2G42120 | DNA polymerase delta small subunit | 1 |
| AT1G10520 | DNA polymerase lambda (POLL) | 1 |
| AT2G32000 | DNA TOPOISOMERASE FAMILY PROTEIN | 1 |
| AT5G63960 | DNA-directed DNA polymerase delta catalytic subunit, putative | 1 |
| | (POLD1) | |
| AT2G27120 | DNA-directed DNA polymerase epsilon catalytic subunit, putative, | 1 |
| | similar to SP\|Q07864 DNA polymerase epsilon, catalytic subunit A (EC | |
| | 2.7.7.7) (DNA polymerase II subunit A) {Homo sapiens}; contains Pfam | |
| | profiles: PF03175 DNA polymerase type B, organellar | |
| AT2G22360 | DNAJ heat shock family protein | 1 |
| AT3G60190 | dynamin-like protein E | 1 |
| AT5G13680 | ELO2 | 1 |
| AT1G12360 | ENCODES A SEC1 PROTEIN AND EXPRESSED THROUGHOUT | 1 |
| | THE PLANT. PHYSICALLY INTERACTS WITH SYNTAXIN1 AND IS | |
| | REQUIRED FOR CYTOKINESIS. | |
| AT3G23580 | ENCODES ONE OF THE 3 RIBONUCLEOTIDE REDUCTASE (RNR) | 1 |
| | SMALL SUBUNIT GENES (RNR2A). FUNCTIONALLY REDUNDANT | |
| | WITH THE RIBONUCLEOTIDE REDUCTASE TSO2. MRNA WAS | |
| | SHOWN TO SPECIFICALLY ACCUMULATE DURING THE S-PHASE | |
| | OF THE CELL CYCLE IN SYNCHRONIZED TOBACCO BY2 CELLS. | |
| | CRITICAL FOR CELL CYCLE PROGRESSION. DNA DAMAGE | |
| | REPAIR AND PLANT DEVELOPMENT. | |
| AT1G50250 | FTSH1 (FTSH PROTEASE 1); ATP-DEPENDENT PEPTIDASE/ | 1 |
| | ATPASE/METALLOPEPTIDASE | |
| AT5G53170 | FTSH11 (FTSH PROTEASE 11); ATP-DEPENDENT PEPTIDASE/ | 1 |
| | ATPASE/METALLOPEPTIDASE | |
| AT2G36250 | FTSZ2-1 (FTSZ HOMOLOG 2-1); STRUCTURAL MOLECULE | 1 |
| AT3G52750 | FTSZ2-2 (FtsZ2-2); structural molecule | 1 |
| AT2G22475 | GRAM DOMAIN-CONTAINING PROTEIN/ABA-RESPONSIVE | 1 |
| | PROTEIN-RELATED | |
| ATMG01250 | HYPOTHETICAL PROTEIN | 1 |
| AT5G08550 | ILP1 (INCREASED LEVEL OF POLYPLOIDY1-1D); TRANSLATION | 1 |
| | REPRESSOR | |
| AT1G30010 | INTRON MATURASE, TYPE II FAMILY PROTEIN | 1 |
| AT4G21270 | kinesin-like protein A (KATA) | 1 |
| AT5G35520 | kinetochore protein-related | 1 |
| AT3G50630 | KRP2 | 1 |
| AT5G48820 | KRP3 | 1 |
| AT2G32710 | KRP4 | 1 |
| AT3G24810 | KRP5 | 1 |
| AT2G26330 | leucine-rich repeat protein kinase, putative (ERECtA), identical to | 1 |
| | uncharacterized receptor protein kinase ERECtA (Arabidopsis thaliana) | |
| | gi: 1389566: dbj: BAA11869; contains Pfam domains PF00560: Leucine | |
| | Rich Repeat and PF00069: Protein kinase domain | |
| AT3G56700 | MALE STERILITY PROTEIN, PUTATIVE | 1 |
| AT4G30820 | MAT1 | 1 |
| AT1G66170 | MMD1 (MALE MEIOCYTE DEATH 1); DNA BINDING | 1 |
| AT4G20900 | MS5 (MALE-STERILE 5) | 1 |
| AT1G79150 | NOC3_DNA replication initiation | 1 |
| AT2G01840 | non-LTR retrotransposon family (LINE), has a 9.6e−34 P-value blast | 1 |
| | match to GB: NP_038607 L1 repeat, Tf subfamily, member 9 | |
| | (LINE-element) (Mus musculus) | |
| AT2G35190 | NPSN11 (NOVEL PLANT SNARE 11); protein transporter | 1 |
| AT1G77620 | NUCLEOSIDE-TRIPHOSPHATASE/NUCLEOTIDE BINDING | 1 |
| AT4G12620 | ORC1 | 1 |
| AT3G44550 | OXIDOREDUCTASE, ACTING ON THE CH-CH GROUP OF DONORS | 1 |
| AT3G44560 | OXIDOREDUCTASE, ACTING ON THE CH-CH GROUP OF DONORS | 1 |
| AT5G10480 | PAS2 (PASTICCINO 2) | 1 |
| AT1G07370 | PCNA1 | 1 |
| AT1G71440 | PFI_TFC E_PFI (PFIFFERLING) | 1 |
| AT2G18040 | PIN1 | 1 |
| AT5G22130 | PNT1 (PEANUT 1); MANNOSYLTRANSFERASE/TRANSFERASE, | 1 |
| | TRANSFERRING GLYCOSYL GROUPS | |
| AT3G20540 | POLGAMMA1 (POLYMERASE GAMMA 1); DNA BINDING/ | 1 |
| | DNA-DIRECTED DNA POLYMERASE | |
| AT4G14713 | PPD1 (PEAPOD 1) | 1 |
| AT1G49180 | protein kinase family protein | 1 |
| AT3G12530 | PSF2 (GINS complex DNA replication) | 1 |
| AT2G17910 | putative non-LTR retroelement reverse transcriptase | 1 |
| AT4G36390 | radical SAM domain-containing protein/TRAM domain-containing | 1 |
| | protein | |
| AT1G63160 | replication factor, putative; similar to GI: 4972952 from (Mus musculus) | 1 |
| | (Mamm. Genome (2000) In press) | |
| AT5G45400 | replication protein, putative, similar to replication protein A 70 kDa | 1 |
| | (Oryza sativa (japonica cultivar-group)) GI: 13536993; contains InterPro | |
| | entry IPR004365: OB-fold nucleic acid binding domain, PF04057: | |
| | Replication factor-A protein 1, N-terminal domain | |
| AT4G19130 | replication protein-related, similar to replication protein A 70 kDa (Oryza | 1 |
| | sativa) GI: 13536993; contains Pfam profile PF00098: Zinc knuckle | |
| AT4G20520 | RNA binding/RNA-directed DNA polymerase | 1 |
| AT2G20580 | RPN1 | 1 |
| AT3G54220 | SCR (SCARECROW); TRANSCRIPTION FACTOR | 1 |
| AT1G14750 | SDS | 1 |
| AT1G22530 | SEC14 cytosolic factor family protein | 1 |
| AT1G34210 | SERK2 (SOMATIC EMBRYOGENESIS RECEPTOR-LIKE KINASE 2); | 1 |
| | KINASE | |
| AT5G52800 | SIMILAR TO PREDICTED: HYPOTHETICAL PROTEIN [RATTUS | 1 |
| | NORVE (GB: XP_001060721.1); SIMILAR TO OS07G0467900 | |
| | [ORYZA SATIVA (JAPONICA CULTIVAR-GR | |
| AT5G58650 | SIMILAR TO UNKNOWN PROTEIN [ARABIDOPSIS THALIANA] | 1 |
| | (TAIR: AT3G47295.1) | |
| AT1G75950 | SKP1 (ARABIDOPSIS SKP1 HOMOLOGUE); ubiquitin-protein ligase | 1 |
| AT3G47460 | SMC2-like condensin, putative/chromosome assembly protein | 1 |
| | homolog; CHROMOSOME ASSEMBLY PROTEIN XCAP-E - Xenopus | |
| | laevis, SWISSPROT: XCPE_XENLA | |
| AT2G02480 | STI (STICHEL); ATP BINDING/DNA-DIRECTED DNA | 1 |
| | POLYMERASE | |
| AT5G07660 | structural maintenance of chromosomes (SMC) family protein | 1 |
| AT3G51770 | tetratricopeptide repeat (TPR)-containing protein | 1 |
| AT5G23070 | thymidine kinase, putative, similar to thymidine kinase (Oryza sativa) | 1 |
| | SWISS-PROT: O81263 | |
| AT3G01780 | TPLATE | 1 |
| AT4G07600 | transposable element gene | 1 |
| AT4G09710 | transposable element gene | 1 |
| AT4G14470 | transposable element gene | 1 |
| AT4G04230 | transposable element gene | 1 |
| AT3G18730 | TSK (TONSOKU) | 1 |
| AT1G22275 | unknown protein | 1 |
| AT5G42270 | VAR1 (VARIEGATED 1); ATP-DEPENDENT PEPTIDASE/ATPASE/ | 1 |
| | METALLOPEPTIDASE | |
| AT2G33880 | WOX9 (STIMPY); transcription factor | 1 |
| AT1G24490 | ALB4 (ALBINA 4) | 0 |
| AT5G28640 | AN3 | 0 |
| AT3G05870 | APC11 | 0 |
| AT2G37630 | AS1/ATMYB91/ATPHAN/MYB91 (ASYMMETRIC LEAVES 1, MYB | 0 |
| | DOMAIN PROTEIN 91); DNA binding/protein homodimerization/ | |
| | transcription factor | |
| AT3G21850 | ASK9 (ARABIDOPSIS SKP1-LIKE 9); ubiquitin-protein ligase | 0 |
| AT4G32200 | ASY2; DNA binding | 0 |
| AT3G33520 | ATARP6; structural constituent of cytoskeleton | 0 |
| AT3G48190 | ataxia-telangiectasia mutated protein AtATM | 0 |
| AT5G05620 | ATGCP2/TUBG2 (GAMMA-TUBULIN); structural molecule | 0 |
| AT3G20475 | ATP binding/damaged DNA binding | 0 |
| AT1G49250 | ATP dependent DNA ligase family protein | 0 |
| AT1G13590 | ATPSK1 (PHYTOSULFOKINE 1 PRECURSOR); growth factor | 0 |
| AT2G22860 | ATPSK2_ATPSK2 (PHYTOSULFOKINE 2 PRECURSOR); growth | 0 |
| | factor | |
| AT5G65870 | ATPSK5 (PHYTOSULFOKINE 5 PRECURSOR); growth factor | 0 |
| AT4G37720 | ATPSK6 (PHYTOSULFOKINE 6 PRECURSOR); growth factor | 0 |
| AT1G14410 | ATWHY1/PTAC1 (A. THALIANA WHIRLY 1); DNA binding/telomeric | 0 |
| | DNA binding | |
| AT5G09790 | ATXR5 (SETDOMAIN GROUP 15); DNA binding | 0 |
| AT3G24730 | catalytic | 0 |
| AT3G48750 | CDKA; 1 | 0 |
| AT3G54180 | CDKB1; 1 | 0 |
| AT5G66130 | cell cycle checkpoint protein-related, weak similarity to cell cycle | 0 |
| | checkpoint protein RAD17 (Homo sapiens) GI: 4102916 | |
| Q94G54 | Cell division control protein 6 | 0 |
| Q9C8M6 | Cell division control protein, putative; 15914-18846 | 0 |
| Q9LFB3 | Cell division-like protein | 0 |
| AT1G53050 | CKL15 | 0 |
| AT3G05050 | CKL8 | 0 |
| AT3G15170 | CUC1 (CUP-SHAPED COTYLEDON1); transcription factor | 0 |
| AT1G20610 | CYCB2; 3 | 0 |
| AT5G48630 | CYCC1; 2 | 0 |
| AT5G10440 | CYCD4; 2 | 0 |
| AT5G02110 | CYCD7; 1 | 0 |
| AT2G01905 | CYCJ18 | 0 |
| Q38818 | Cyclin 2 | 0 |
| AT2G44740 | CYCLIN FAMILY PROTEIN, SIMILAR TO CYCLIN 2 | 0 |
| | (TRYPANOSOMA BRUCEI) GI: 7339572, CYCLIN 6 (TRYPANOSOMA | |
| | CRUZI) GI: 12005317; CONTAINS PFAM PROFILE PF00134: | |
| | CYCLIN, N-TERMINAL DOMAIN\|CHR2: 18449064-18450471 | |
| | REVERSE\|ALIASES: F16B22.23 | |
| AT5G07450 | CYCLIN FAMILY PROTEIN, SIMILAR TO CYCLIN 2 | 0 |
| | (TRYPANOSOMA BRUCEI) GI: 7339572, CYCLIN 6 (TRYPANOSOMA | |
| | CRUZI) GI: 12005317; CONTAINS PFAM PROFILE PF00134: | |
| | CYCLIN, N-TERMINAL DOMAIN\|CHR5: 2358353-2359309 | |
| | REVERSE\|ALIASES: T2I1.160, T2I1_160 | |
| AT1G22840 | cytochrome c, putative | 0 |
| AT4G26701 | DNA binding/DNA topoisomerase type I | 0 |
| AT3G15960 | DNA mismatch repair MutS family protein | 0 |
| AT5G22110 | DNA POLYMERASE EPSILON SUBUNIT B FAMILY, CONTAINS | 0 |
| | PFAM PROFILE: PF04042 DNA POLYMERASE EPSILON SUBUNIT | |
| | B\|CHR5: 7330828-7334181 REVERSE\|ALIASES: NONE | |
| AT5G64520 | DNA repair protein-related | 0 |
| AT1G63990 | DNA topoisomerase VIA, putative (SPO11-2) | 0 |
| AT4G39960 | DNAJ heat shock family protein | 0 |
| AT5G62450 | DOMINO1 | 0 |
| AT5G03415 | DPb | 0 |
| AT5G50320 | ELO3 | 0 |
| Q9FMR4 | Emb\|CAB92117.1 | 0 |
| AT1G24590 | ENCODES A MEMBER OF THE ERF (ETHYLENE RESPONSE | 0 |
| | FACTOR) SUBFAMILY B-1 OF ERF/AP2 TRANSCRIPTION FACTOR | |
| | FAMILY. THE PROTEIN CONTAINS ONE AP2 DOMAIN. THERE ARE | |
| | 15 MEMBERS IN THIS SUBFAMILY INCLUDING ATERF-3. ATERF-4. | |
| | ATERF-7. AND LEAFY PETIOLE. THIS GENE FUNCTIONS IN THE | |
| | REGENERATION OF SHOOTS IN TISSUE CULTURE. PROBABLY | |
| | THROUGH TRANSCRIPTIONAL REGULATION OF CUC1. MAY | |
| | ALSO BE INVOLVED IN ACTIVATION OF THE CELL CYCLE VIA | |
| | CYCD1; 1. | |
| AT5G26680 | ENDONUCLEASE, PUTATIVE, SIMILAR TO SWISS-PROT: P39748 | 0 |
| | FLAP ENDONUCLEASE-1 (MATURATION FACTOR 1) (MF1) (HOMO | |
| | SAPIENS)\|CHR5: 9311885-9315478 REVERSE\|ALIASES: NONE | |
| AT1G22260 | expressed protein | 0 |
| Q9M874 | F16B3.31 protein | 0 |
| Q9FX75 | F19K19.10 protein | 0 |
| Q9FYF5 | F1N21.20 | 0 |
| O81303 | F6N15.14 protein (Putative BRCA2 homolog) | 0 |
| AT2G14650 | F9D12.11 protein (contains similarity to reverse transcriptases) | 0 |
| AT3G24140 | FMA (FAMA); DNA binding/transcription activator/transcription factor | 0 |
| AT2G22942 | growth factor | 0 |
| AT5G42080 | GTP-binding protein/phragmoplastin, putative | 0 |
| AT1G47840 | hexokinase, putative | 0 |
| AT5G03410 | histidyl-tRNA synthetase - like protein | 0 |
| AT2G44950 | HUB1 | 0 |
| AT2G44960 | HUB1 | 0 |
| AT3G52630 | hypothetical protein | 0 |
| AT1G74350 | intron maturase, type II family protein | 0 |
| AT5G03150 | JKD (JACKDAW); nucleic acid binding/protein binding/protein | 0 |
| | homodimerization/transcription factor/zinc ion binding | |
| Q2V0Z5 | Kinetochore protein | 0 |
| AT2G30410 | KIS (KIESEL); unfolded protein binding | 0 |
| ATMG00520 | MATR | 0 |
| AT5G22260 | MS1 (MALE STERILITY 1); DNA binding | 0 |
| AT3G11980 | MS2 (MALE STERILITY 2) | 0 |
| AT2G01120 | ORC4 | 0 |
| AT4G27650 | PEL1 (PELOTA); translation release factor | 0 |
| AT2G01500 | PFS2 (PRETTY FEW SEEDS 2); transcription factor | 0 |
| AT3G44735 | PSK1 (PHYTOSULFOKINE 3 PRECURSOR); growth factor | 0 |
| Q9M8S3 | Putative CCHC-type zinc finger protein | 0 |
| AT4G39920 | putative protein | 0 |
| AT5G22370 | putative protein; similar to unknown protein (emb\|CAB92117.1) | 0 |
| AT2G07660 | putative retroelement pol polyprotein | 0 |
| O80896 | Putative uncharacterized protein At2g32590 | 0 |
| Q9M013 | Putative uncharacterized protein F7A7_150 | 0 |
| O82745 | Putative uncharacterized protein F7H19.150 (Putative uncharacterized | 0 |
| | protein AT4g22970) | |
| Q9C953 | Putative uncharacterized protein T7P1.14 | 0 |
| AT4G20050 | QRT3 (QUARTET 3) | 0 |
| AT2G06510 | replication protein, putative similar to replication protein A 70 kDa | 0 |
| AT4G29090 | reverse transcriptase, putative/RNA-dependent DNA polymerase, | 0 |
| | putative | |
| AT2G25100 | ribonuclease HII family protein | 0 |
| AT5G40942 | RNR2B | 0 |
| AT2G36985 | ROT4 (ROTUNDIFOLIA4) | 0 |
| AT4G18730 | RPL16B (ribosomal protein L16B); structural constituent of ribosome | 0 |
| AT1G17235 | RTFL11 (ROTUNDIFOLIA LIKE 11) | 0 |
| AT4G37650 | SHR (SHORT ROOT); transcription factor | 0 |
| AT5G04470 | SIM | 0 |
| AT3G55490 | similar to TTN10 (TITAN 10) | 0 |
| AT5G62440 | similar to unnamed protein product [Vitis vinifera] (GB: CAO64547.1) | 0 |
| Q9FGA0 | Similarity to glucose inhibited division protein B | 0 |
| AT1G20330 | SMT2 (STEROL METHYLTRANSFERASE 2) | 0 |
| AT4G27330 | SPL (SPOROCYTELESS) | 0 |
| AT1G02065 | SPL8 (SQUAMOSA PROMOTER BINDING PROTEIN-LIKE 8); DNA | 0 |
| | binding | |
| AT3G07800 | thymidine kinase, putative, similar to thymidine kinase (Oryza sativa) | 0 |
| | SWISS-PROT: O81263 | |
| AT2G05610 | transposable element gene | 0 |
| AT4G08830 | transposable element gene | 0 |
| AT4G10580 | transposable element gene | 0 |
| Q9FLP1 | Trigger factor-like protein | 0 |
| AT1G19080 | TTN10 (TITAN 10) | 0 |
| AT3G61650 | TUBG1 (GAMMA-TUBULIN); STRUCTURAL MOLECULE | 0 |
| AT1G05560 | UGT1 (UDP-glucosyl transferase 75B1); UDP-glycosyltransferase/ | 0 |
| | transferase, transferring glycosyl groups | |
| AT1G47200 | WPP2 (WPP domain protein 2) | 0 |
| AT5G57450 | XRCC3\|DNA REPAIR FAMILY PROTEIN, CONTAINS SIMILARITY | 0 |
| | TO SWISS-PROT: O43542 DNA-REPAIR PROTEIN XRCC3 (X-RAY | |
| | REPAIR CROSS-COMPLEMENTING PROTEIN 3) (HOMO SAPIENS)\| | |
| | CHR5: 23290319-23292202 REVERSE\|ALIASES: ATXRCC3, | |
| | MUA2.3, MUA2_3 | |

TABLE 6
New candidate cell cycle proteins.

| | | |
|---|---|---|
| Locus | Description | # of motifs |

Overview of 40 putative new cell cycle proteins identified in the core dataset containing
at least two of the cell cycle-related features listed in Table S4. The number of identified
features per protein is shown.

| | | |
|---|---|---|
| AT2G44580 | DCC1 | 6 |
| AT3G57860 | UVI4-like | 5 |
| AT4G38900 | bZIP protein | 5 |
| AT1G32310 | F27G20.14 | 5 |
| AT4G17020 | transcription factor-related | 4 |
| AT5G09900 | EMB2107, RPN5A, MSA | 4 |
| AT5G40460 | expressed | 4 |
| AT3G49240 | EMB1796 | 4 |
| AT2G17350 | similar to hypothetical protein [Vitis vinifera] (GB: CAN62847.1) | 4 |
| AT5G24690 | similar to RER1 (RETICULATA-RELATED 1) [Arabidopsis thaliana] | 4 |
| | (TAIR: AT5G22790.1) | |
| AT4G25550 | M7J2_80 | 4 |
| AT3G15180 | proteasome-related | 3 |
| AT2G36130 | peptidyl-prolyl cis-trans isomerase, putative/cyclophilin, putative/rotamase | 3 |
| AT3G16060 | kinesin motor family protein | 3 |
| AT3G10180 | kinesin motor protein-related | 3 |
| AT5G65460 | KCA2 | 3 |
| AT5G05780 | RPN8A, AE3, ATHMOV34 | 3 |
| AT1G06070 | bZIP transcription factor, putative (bZIP69) | 3 |
| AT1G71380 | ATGH9B3, ATCEL3 | 3 |
| AT3G05530 | ATS6A.2, RPT5A | 2 |
| AT1G01880 | DNA repair protein, putative | 2 |
| AT4G34150 | C2 domain-containing protein | 2 |
| AT5G13030 | similar to hypothetical protein Osl_021963 [Oryza sativa] (GB: EAZ00731.1) | 2 |
| AT4G14310 | DL3195C | 2 |
| AT5G26360 | chaperonin, putative | 2 |
| AT5G49510 | VHL binding protein, putative/prefoldin, putative | 2 |
| AT3G20050 | ATTCP-1 | 2 |
| AT1G29150 | RPN6, ATS9 | 2 |
| AT3G15970 | Ran-binding protein 1 domain-containing protein/RanBP1 domain-containing | 2 |
| | protein | |
| AT4G28230 | similar to unnamed protein product [Vitis vinifera] (GB: CAO41238.1) | 2 |
| AT5G23540 | 26S proteasome regulatory subunit, putative | 2 |
| AT5G41190 | similar to hypothetical protein [Vitis vinifera] (GB: CAN66411.1) | 2 |
| AT2G03820 | nonsense-mediated mRNA decay NMD3 family protein | 2 |
| AT1G20480 | 4-coumarate--CoA ligase family protein/4-coumaroyl-CoA synthase family | 2 |
| | protein | |
| AT1G09270 | importin alpha-1 subunit, putative (IMPA4) | 2 |
| AT1G23190 | phosphoglucomutase, cytoplasmic, putative/glucose phosphomutase, putative | 2 |
| AT2G06990 | HEN2 | 2 |
| AT5G58290 | RPT3 | 2 |
| AT5G20920 | EMB1401, EIF2 BETA | 2 |
| AT1G20200 | EMB2719 | 2 |

Overview of 82 putative new cell cycle proteins identified in the non-core dataset
containing at least two of the cell cycle-related features listed in Table S4. The number of
identified features per protein is shown.

| | | |
|---|---|---|
| AT5G18620 | CHR17 | 6 |
| AT4G38780 | splicing factor, putative | 5 |
| AT1G01970 | pentatricopeptide (PPR) repeat-containing protein | 5 |
| AT4G38900 | bZIP protein | 5 |
| AT4G17020 | transcription factor-related | 4 |
| AT2G46020 | ATBRM, CHR2, BRM | 4 |
| AT5G10800 | RNA recognition motif (RRM)-containing protein | 4 |
| AT5G40460 | expressed | 4 |
| AT1G36160 | AT-ACC1, EMB22, GK, PAS3, ACC1 | 4 |
| AT1G77180 | chromatin protein family | 4 |
| AT1G16520 | similar to unknown protein [Arabidopsis thaliana] (TAIR: AT1G56080.1) | 4 |
| AT3G49240 | EMB1796 | 4 |
| AT4G24680 | similar to unnamed protein product [Vitis vinifera] (GB: CAO64289.1) | 4 |
| AT2G25730 | binding/heme binding | 4 |
| AT5G38480 | RCI1, GRF3 | 4 |
| AT4G25550 | M7J2_80 | 4 |
| AT3G05190 | aminotransferase class IV family protein | 4 |
| AT4G35740 | RecQI3 | 4 |
| AT3G62240 | zinc finger (C2H2 type) family protein | 3 |
| AT3G45970 | EXPL1, ATEXPL1, ATHEXP BETA 2.1, ATEXLA1 | 3 |
| AT3G56690 | CIP111 | 3 |
| AT3G56860 | UBA2A | 3 |
| AT3G01280 | porin, putative | 3 |
| AT3G20390 | endoribonuclease L-PSP family protein | 3 |
| AT5G03740 | HDT3, HD2C | 3 |
| AT5G25210 | similar to unknown protein [Arabidopsis thaliana] (TAIR: AT4G32030.1) | 3 |
| AT1G48920 | ATNUC-L1, PARL1 | 3 |
| AT5G58150 | leucine-rich repeat transmembrane protein kinase, putative | 3 |
| AT1G17340 | phosphoinositide phosphatase family protein | 3 |
| AT1G23860 | RSZP21, SRZ21, SRZ-21 | 3 |
| AT2G16950 | protein transporter | 3 |
| AT1G06070 | bZIP transcription factor, putative (bZIP69) | 3 |
| AT1G71380 | ATGH9B3, ATCEL3 | 3 |
| AT2G44350 | CSY4, ATCS | 3 |
| AT1G30860 | protein binding/zinc ion binding | 3 |
| AT3G47930 | ATGLDH | 3 |
| AT1G03060 | WD-40 repeat family protein/beige-related | 3 |
| AT4G21860 | MSRB2 | 3 |
| AT2G30470 | HSI2 | 2 |
| AT1G80720 | mitochondrial glycoprotein family protein/MAM33 family protein | 2 |
| AT1G09760 | U2A′ | 2 |
| AT1G52360 | coatomer protein complex, subunit beta 2 (beta prime), putative | 2 |
| AT1G22300 | GF14 EPSILON, GRF10 | 2 |
| AT3G13640 | ATRLI1 | 2 |
| AT5G12140 | ATCYS1 | 2 |
| AT1G66340 | EIN1, ETR, ETR1 | 2 |
| AT1G42440 | similar to unknown protein [Arabidopsis thaliana] (TAIR: AT1G06720.1) | 2 |
| AT4G09320 | NDPK1 | 2 |
| AT5G54900 | ATRBP45A | 2 |
| AT1G09100 | RPT5B | 2 |
| AT1G52730 | transducin family protein/WD-40 repeat family protein | 2 |
| AT3G49430 | SRP34A | 2 |
| AT3G53880 | aldo/keto reductase family protein | 2 |
| AT4G16630 | DEAD/DEAH box helicase, putative (RH28) | 2 |
| AT2G03820 | nonsense-mediated mRNA decay NMD3 family protein | 2 |
| AT1G23190 | phosphoglucomutase, cytoplasmic, putative/glucose phosphomutase, | 2 |
| | putative | |
| AT3G01540 | ATDRH1, DRH1 | 2 |
| AT5G20920 | EMB1401, EIF2 BETA | 2 |
| AT4G14310 | DL3195C | 2 |
| AT1G09430 | ACLA-3 | 2 |
| AT2G33340 | transducin family protein/WD-40 repeat family protein | 2 |
| AT1G01880 | DNA repair protein, putative | 2 |
| AT5G62350 | invertase/pectin methylesterase inhibitor family protein/DC 1.2 homolog | 2 |
| | (FL5-2I22) | |
| AT4G34150 | C2 domain-containing protein | 2 |
| AT5G46780 | VQ motif-containing protein | 2 |
| AT5G26360 | chaperonin, putative | 2 |
| AT4G38440 | similar to hypothetical protein [Vitis vinifera] (GB: CAN83259.1) | 2 |
| AT3G29800 | AAA-type ATPase family | 2 |
| AT5G58290 | RPT3 | 2 |
| AT3G20050 | ATTCP-1 | 2 |
| AT1G19520 | NFD5 | 2 |
| AT4G26870 | aspartyl-tRNA synthetase, putative/aspartate--tRNA ligase, putative | 2 |
| AT1G56080 | similar to unknown protein [Arabidopsis thaliana] (TAIR: AT1G16520.1) | 2 |
| AT5G12380 | annexin, putative | 2 |
| AT4G28230 | similar to unnamed protein product [Vitis vinifera] (GB: CAO41238.1) | 2 |
| AT2G06210 | VIP6, ELF8 | 2 |
| ATCG00480 | ATPB | 2 |
| AT5G02530 | RNA and export factor-binding protein, putative | 2 |
| AT1G31760 | SWIB complex BAF60b domain-containing protein | 2 |
| AT5G06680 | ATSPC98, SPC98 | 2 |
| AT2G16570 | ATASE1, ATASE | 2 |
| AT1G62390 | octicosapeptide/Phox/Bem1p (PB1) domain-containing protein/ | 2 |
| | tetratricopeptide repeat (TPR)-containing protein | |
| AT1G43800 | acyl-(acyl-carrier-protein) desaturase, putative/stearoyl-ACP desaturase, | 2 |
| | putative | |

TABLE 7
Overview of microarray experiments used to calculate the transcript PCCs.
List of experiments used to build an Arabidopsis ATH1 micro-array compendium of 518
experiments focused on cell cycle or plant growth and development used to calculate the
transcript Pearson correlation coefficients.

| Description | Reference |
|---|---|
| co-2; 3 days long day condition; apical region; | AtGenExpress; /share/nasc/AtGenExpress_dev_series_mutants_and_other_ecotypes/ |
| rosette stage | CEL/co-3-1.CEL |
| Col-0; 3 days long day condition; apical region; | AtGenExpress; /share/nasc/AtGenExpress_dev_series_mutants_and_other_ecotypes/ |
| rosette stage | CEL/Col-3-1.CEL |
| ft-2; 3 days long day condition; apical region; | AtGenExpress; /share/nasc/AtGenExpress_dev_series_mutants_and_other_ecotypes/ |
| rosette stage | CEL/ft-3-1.CEL |
| Ler; 3 days long day condition; apical region; | AtGenExpress; /share/nasc/AtGenExpress_dev_series_mutants_and_other_ecotypes/ |
| rosette stage | CEL/Ler-3-1.CEL |
| lfy-12; 3 days long day condition; apical region; | AtGenExpress; /share/nasc/AtGenExpress_dev_series_mutants_and_other_ecotypes/ |
| rosette stage | CEL/lfy-3-1.CEL |
| co-2; 5 days long day condition; apical region; | AtGenExpress; /share/nasc/AtGenExpress_dev_series_mutants_and_other_ecotypes/ |
| rosette stage | CEL/co-5-1.CEL |
| Col-0; 5 days long day condition; apical region; | AtGenExpress; /share/nasc/AtGenExpress_dev_series_mutants_and_other_ecotypes/ |
| rosette stage | CEL/Col-5-1.CEL |
| ft-2; 5 days long day condition; apical region; | AtGenExpress; /share/nasc/AtGenExpress_dev_series_mutants_and_other_ecotypes/ |
| rosette stage | CEL/ft-5-1.CEL |
| Ler; 5 days long day condition; apical region; | AtGenExpress; /share/nasc/AtGenExpress_dev_series_mutants_and_other_ecotypes/ |
| rosette stage | CEL/Ler-5-1.CEL |
| lfy-12; 5 days long day condition; apical region; | AtGenExpress; /share/nasc/AtGenExpress_dev_series_mutants_and_other_ecotypes/ |
| rosette stage | CEL/lfy-5-1.CEL |
| co-2; 7 days long day condition; apical region; | AtGenExpress; /share/nasc/AtGenExpress_dev_series_mutants_and_other_ecotypes/ |
| rosette stage | CEL/co-7-1.CEL |
| Col-0; 7 days long day condition; apical region; | AtGenExpress; /share/nasc/AtGenExpress_dev_series_mutants_and_other_ecotypes/ |
| rosette stage | CEL/Col-7-1.CEL |
| ft-2; 7 days long day condition; apical region; | AtGenExpress; /share/nasc/AtGenExpress_dev_series_mutants_and_other_ecotypes/ |
| rosette stage | CEL/ft-7-1.CEL |
| Ler; 7 days long day condition; apical region; | AtGenExpress; /share/nasc/AtGenExpress_dev_series_mutants_and_other_ecotypes/ |
| rosette stage | CEL/Ler-7-1.CEL |
| lfy-12; 7 days long day condition; apical region; | AtGenExpress; /share/nasc/AtGenExpress_dev_series_mutants_and_other_ecotypes/ |
| rosette stage | CEL/lfy-7-1.CEL |
| co-2; no treatment; apical region; rosette stage | AtGenExpress; /share/nasc/AtGenExpress_dev_series_mutants_and_other_ecotypes/ |
| | CEL/co-0-1.CEL |
| Col-0; no treatment; apical region; rosette stage | AtGenExpress; /share/nasc/AtGenExpress_dev_series_mutants_and_other_ecotypes/ |
| | CEL/Col-0-1.CEL |
| ft-2; no treatment; apical region; rosette stage | AtGenExpress; /share/nasc/AtGenExpress_dev_series_mutants_and_other_ecotypes/ |
| | CEL/ft-0-1.CEL |
| Ler; no treatment; apical region; rosette stage | AtGenExpress; /share/nasc/AtGenExpress_dev_series_mutants_and_other_ecotypes/ |
| | CEL/Ler-0-1.CEL |
| lfy-12; no treatment; apical region; rosette stage | AtGenExpress; /share/nasc/AtGenExpress_dev_series_mutants_and_other_ecotypes/ |
| | CEL/lfy-0-1.CEL |
| Wt roots; 7 days; continuous light; soil | AtGenExpress; /share/nasc/AtGenExpress_dev_series_roots/ |
| | CEL/ATGE_3_A. |
| | CEL |
| Wt roots; 17 days continuous light soil | AtGenExpress; /share/nasc/AtGenExpress_dev_series_roots/ |
| | CEL/ATGE_9_A. |
| | CEL |
| Wt root; 15 days long day (16/8) 1x MS agar, 1% | AtGenExpress; /share/nasc/AtGenExpress_dev_series_roots/ |
| sucrose | CEL/ATGE_93_A. |
| | CEL |
| Wt root; 8 days continuous light 1x MS agar | AtGenExpress; /share/nasc/AtGenExpress_dev_series_roots/ |
| | CEL/ATGE_94_A. |
| | CEL |
| Wt root; 8 days continuous light 1x MS agar, 1% sucrose | AtGenExpress; /share/nasc/AtGenExpress_dev_series_roots/ |
| | CEL/ATGE_95_A. |
| | CEL |
| Wt root; 21 days continuous light 1x MS agar | AtGenExpress; /share/nasc/AtGenExpress_dev_series_roots/ |
| | CEL/ATGE_98_A. |
| | CEL |
| Wt root; 21 days continuous light 1x MS agar, 1% | AtGenExpress; /share/nasc/AtGenExpress_dev_series_roots/ |
| sucrose | CEL/ATGE_99_A. |
| | CEL |
| Wt seedling, green parts 21 days continuous light 1x MS | AtGenExpress; /share/nasc/AtGenExpress_dev_series_seedlings_and_whole_plants/ |
| agar | CEL/ATGE_100_A.CEL |
| Wt seedling, green parts 21 days continuous light 1x MS | AtGenExpress; /share/nasc/AtGenExpress_dev_series_seedlings_and_whole_plants/ |
| agar, 1% sucrose | CEL/ATGE_101_A.CEL |
| Wt developmental drift, entire rosette after transition | AtGenExpress; /share/nasc/AtGenExpress_dev_series_seedlings_and_whole_plants/CEL/ |
| to flowering, but before bolting 21 days continuous | ATGE_22_A.CEL |
| light soil | |
| Wt as above 22 days continuous light soil | AtGenExpress; /share/nasc/AtGenExpress_dev_series_seedlings_and_whole_plants/ |
| | CEL/ATGE_23_A.CEL |
| Wt as above 23 days continuous light soil | AtGenExpress; /share/nasc/AtGenExpress_dev_series_seedlings_and_whole_plants/ |
| | CEL/ATGE_24_A.CEL |
| Wt seedling, green parts; 7 days; continuous light; soil | AtGenExpress; /share/nasc/AtGenExpress_dev_series_seedlings_and_whole_plants/ |
| | CEL/ATGE_7_A2.CEL |
| Wt seedling, green parts 8 days continuous light 1x MS | AtGenExpress; /share/nasc/AtGenExpress_dev_series_seedlings_and_whole_plants/ |
| agar | CEL/ATGE_96_A.CEL |
| Wt seedling, green parts 8 days continuous light 1x MS | AtGenExpress; /share/nasc/AtGenExpress_dev_series_seedlings_and_whole_plants/ |
| agar, 1% sucrose | CEL/ATGE_97_A.CEL |
| Wt siliques, w/ seeds stage 3; mid globular to early | AtGenExpress; /share/nasc/AtGenExpress_dev_series_siliques_and_seeds/ |
| heart embryos 8 wk long day (16/8) soil | CEL/ATGE_76_B.CEL |
| Wt siliques, w/ seeds stage 4; early to late heart embryos | AtGenExpress; /share/nasc/AtGenExpress_dev_series_siliques_and_seeds/ |
| 8 wk long day (16/8) soil | CEL/ATGE_77_D.CEL |
| Wt siliques, w/ seeds stage 5; late heart to mid torpedo | AtGenExpress; /share/nasc/AtGenExpress_dev_series_siliques_and_seeds/ |
| embryos 8 wk long day (16/8) soil | CEL/ATGE_78_D.CEL |
| Wt seeds, stage 6, w/o siliques; mid to late torpedo | AtGenExpress; /share/nasc/AtGenExpress_dev_series_siliques_and_seeds/ |
| embryos 8 wk long day (16/8) soil | CEL/ATGE_79_A.CEL |
| Wt seeds, stage 7, w/o siliques; late torpedo to early | AtGenExpress; /share/nasc/AtGenExpress_dev_series_siliques_and_seeds/ |
| walking-stick embryos 8 wk long day (16/8) soil | CEL/ATGE_81_A.CEL |
| Wt seeds, stage 8, w/o siliques; walking-stick to early | AtGenExpress; /share/nasc/AtGenExpress_dev_series_siliques_and_seeds/ |
| curled cotyledons | CEL/ATGE_82_A.CEL |
| Wt seeds, stage 9, w/o siliques; curled cotyledons to | AtGenExpress; /share/nasc/AtGenExpress_dev_series_siliques_and_seeds/ |
| early green cotyledons embryos; 8 wk long day | CEL/ATGE_83_A.CEL |
| (16/8) soil | |
| Wt seeds, stage 10, w/o siliques; green cotyledons | AtGenExpress; /share/nasc/AtGenExpress_dev_series_siliques_and_seeds/ |
| embryos 8 wk long day (16/8) soil | CEL/ATGE_84_A.CEL |
| Wt flowers stage 9, 21+ days continuous light soil | AtGenExpress; /share/nasc/AtGenExpress_Developmental_series_flowers_and_pollen/ |
| | CEL/ATGE_31_A2.CEL |
| Wt flowers stage 10/11, 21+ days continuous light soil | AtGenExpress; /share/nasc/AtGenExpress_Developmental_series_flowers_and_pollen/ |
| | CEL/ATGE_32_A2.CEL |
| Wt flowers stage 12, 21+ days continuous light soil | AtGenExpress; /share/nasc/AtGenExpress_Developmental_series_flowers_and_pollen/ |
| | CEL/ATGE_33_A.CEL |
| Wt flowers stage 12, sepals 21+ days continuous light | AtGenExpress; /share/nasc/AtGenExpress_Developmental_series_flowers_and_pollen/ |
| soil | CEL/ATGE_34_A.CEL |
| Wt flowers stage 12, petals 21+ days continuous light | AtGenExpress; /share/nasc/AtGenExpress_Developmental_series_flowers_and_pollen/ |
| soil | CEL/ATGE_35_A.CEL |
| Wt flowers stage 12, stamens 21+ days continuous light | AtGenExpress; /share/nasc/AtGenExpress_Developmental_series_flowers_and_pollen/ |
| soil | CEL/ATGE_36_A.CEL |
| Wt flowers stage 12, carpels 21+ days continuous light | AtGenExpress; /share/nasc/AtGenExpress_Developmental_series_flowers_and_pollen/ |
| soil | CEL/ATGE_37_A.CEL |
| Wt flowers stage 15, 21+ days continuous light soil | AtGenExpress; /share/nasc/AtGenExpress_Developmental_series_flowers_and_pollen/ |
| | CEL/ATGE_39_A.CEL |
| Wt flowers stage 15, pedicels 21+ days continuous | AtGenExpress; /share/nasc/AtGenExpress_Developmental_series_flowers_and_pollen/ |
| light soil | CEL/ATGE_40_A.CEL |
| Wt flowers stage 15, sepals 21+ days continuous light | AtGenExpress; /share/nasc/AtGenExpress_Developmental_series_flowers_and_pollen/ |
| soil | CEL/ATGE_41_A.CEL |
| Wt flowers stage 15, petals 21+ days continuous light | AtGenExpress; /share/nasc/AtGenExpress_Developmental_series_flowers_and_pollen/ |
| soil | CEL/ATGE_42_B.CEL |
| Wt flowers stage 15, stamen 21+ days continuous light | AtGenExpress; /share/nasc/AtGenExpress_Developmental_series_flowers_and_pollen/ |
| soil | CEL/ATGE_43_A.CEL |
| Wt flowers stage 15, carpels 21+ days continuous light | AtGenExpress; /share/nasc/AtGenExpress_Developmental_series_flowers_and_pollen/ |
| soil | CEL/ATGE_45_A.CEL |
| Wt mature pollen 6 wk continuous light soil | AtGenExpress; /share/nasc/AtGenExpress_Developmental_series_flowers_and_pollen/ |
| | CEL/ATGE_73_A.CEL |
| Wt flower 28 days long day (16/8) soil | AtGenExpress; /share/nasc/AtGenExpress_Developmental_series_flowers_and_pollen/ |
| | CEL/ATGE_92_A.CEL |
| clv3-7 flower stage 12; multi-carpel gynoeceum; enlarged | AtGenExpress; /share/nasc/AtGenExpress_Developmental_series_flowers_and_pollen/ |
| meristem; increased organ number; 21+ days continuous | CEL/ATGE_53_A.CEL |
| light soil | |
| lfy-12 flower stage 12; shoot characteristics; most organs | AtGenExpress; /share/nasc/AtGenExpress_Developmental_series_flowers_and_pollen/ |
| leaf-like 21+ days continuous light soil | CEL/ATGE_54_A.CEL |
| ap1-15 flower stage 12; sepals replaced by leaf-like | AtGenExpress; /share/nasc/AtGenExpress_Developmental_series_flowers_and_pollen/ |
| organs, petals mostly lacking, 2° flowers; 21+ days | CEL/ATGE_55_A.CEL |
| continuous light soil | |
| ap2-6 flower stage 12; no sepals or petals 21+ days | AtGen Express; /share/nasc/AtGenExpress_Developmental_series_flowers_and_pollen/ |
| continuous light soil | CEL/ATGE_56_A.CEL |
| ap3-6 flower stage 12; no petals or stamens 21+ days | AtGenExpress; /share/nasc/AtGenExpress_Developmental_series_flowers_and_pollen/ |
| continuous light soil | CEL/ATGE_57_A.CEL |
| ag-12 flower stage 12; no stamens or carpels 21+ days | AtGenExpress; /share/nasc/AtGenExpress_Developmental_series_flowers_and_pollen/ |
| continuous light soil | CEL/ATGE_58_A.CEL |
| ufo-1 flower stage 12; filamentous organs in whorls two | AtGenExpress; /share/nasc/AtGenExpress_Developmental_series_flowers_and_pollen/ |
| and three 21+ days continuous light soil | CEL/ATGE_59_A.CEL |
| Wt cotyledons; 7 days; continuous light; soil | AtGenExpress; /share/nasc/AtGenExpress_Developmental_series_leaves/ |
| | CEL/ATGE_1_A.CEL |
| Wt rosette leaf #4, 1 cm long 10 days continuous light | AtGenExpress; /share/nasc/AtGenExpress_Developmental_series_leaves/ |
| soil | CEL/ATGE_10_A.CEL |
| gl1-T rosette leaf #4, 1 cm long 10 days continuous | AtGenExpress; /share/nasc/AtGenExpress_Developmental_series_leaves/ |
| light soil | CEL/ATGE_11_A.CEL |
| Wt rosette leaf #2, 17 days continuous light soil | AtGenExpress; /share/nasc/AtGenExpress_Developmental_series_leaves/ |
| | CEL/ATGE_12_A.CEL |
| Wt rosette leaf #4, 17 days continuous light soil | AtGenExpress; /share/nasc/AtGenExpress_Developmental_series_leaves/ |
| | CEL/ATGE_13_A.CEL |
| Wt rosette leaf #6, 17 days continuous light soil | AtGenExpress; /share/nasc/AtGenExpress_Developmental_series_leaves/ |
| | CEL/ATGE_14_A.CEL |
| Wt rosette leaf #8, 17 days continuous light soil | AtGenExpress; /share/nasc/AtGenExpress_Developmental_series_leaves/ |
| | CEL/ATGE_15_A.CEL |
| Wt rosette leaf #10, 17 days continuous light soil | AtGenExpress; /share/nasc/AtGenExpress_Developmental_series_leaves/ |
| | CEL/ATGE_16_A.CEL |
| Wt rosette leaf #12, 17 days continuous light soil | AtGenExpress; /share/nasc/AtGenExpress_Developmental_series_leaves/ |
| | CEL/ATGE_17_A.CEL |
| gl1-T rosette leaf #12, 17 days continuous light soil | AtGenExpress; /share/nasc/AtGenExpress_Developmental_series_leaves/ |
| | CEL/ATGE_18_A.CEL |
| Wt leaf 7, petiole 17 days continuous light soil | AtGenExpress; /share/nasc/AtGenExpress_Developmental_series_leaves/ |
| | CEL/ATGE_19_A.CEL |
| Wt leaf 7, proximal half 17 days continuous light soil | AtGenExpress; /share/nasc/AtGenExpress_Developmental_series_leaves/ |
| | CEL/ATGE_20_A.CEL |
| Wt leaf 7, distal half 17 days continuous light soil | AtGenExpress; /share/nasc/AtGenExpress_Developmental_series_leaves/ |
| | CEL/ATGE_21_A.CEL |
| Wt senescing leaves 35 days continuous light soil | AtGenExpress; /share/nasc/AtGenExpress_Developmental_series_leaves/ |
| | CEL/ATGE_25_A.CEL |
| Wt cauline leaves 21+ days continuous light soil | AtGenExpress; /share/nasc/AtGenExpress_Developmental_series_leaves/ |
| | CEL/ATGE_26_A.CEL |
| Wt leaves 1 + 2; 7 days; continuous light; soil | AtGenExpress; /share/nasc/AtGenExpress_Developmental_series_leaves/ |
| | CEL/ATGE_5_A.CEL |
| Wt vegetative rosette 7 days short day (10/14) soil | AtGenExpress; /share/nasc/AtGenExpress_Developmental_series_leaves/ |
| | CEL/ATGE_87_A.CEL |
| Wt vegetative rosette 14 days short day (10/14) soil | AtGenExpress; /share/nasc/AtGenExpress_Developmental_series_leaves/ |
| | CEL/ATGE_89_A.CEL |
| Wt vegetative rosette 21 days short day (10/14) soil | AtGenExpress; /share/nasc/AtGenExpress_Developmental_series_leaves/ |
| | CEL/ATGE_90_A.CEL |
| Wt leaf 15 days long day (16/8) 1x MS agar, 1% sucrose | AtGenExpress; /share/nasc/AtGenExpress_Developmental_series_leaves/ |
| | CEL/ATGE_91_A.CEL |
| Wt hypocotyl; 7 days; continuous light; soil | AtGenExpress; /share/nasc/AtGenExpress_Developmental_series_shoots_and_stems/ |
| | CEL/ATGE_2_A.CEL |
| Wt stem, 2nd internode 21+ days continuous light soil | AtGenExpress; /share/nasc/AtGenExpress_Developmental_series_shoots_and_stems/ |
| | CEL/ATGE_27_A.CEL |
| Wt 1st node 21+ days continuous light soil | AtGenExpress; /share/nasc/AtGenExpress_Developmental_series_shoots_and_stems/ |
| | CEL/ATGE_28_A2.CEL |
| Wt shoot apex, inflorescence (after bolting) 21 days | AtGenExpress; /share/nasc/AtGenExpress_Developmental_series_shoots_and_stems/ |
| continuous light soil | CEL/ATGE_29_A2.CEL |
| Wt shoot apex, vegetative + young leaves; 7 days; | AtGenExpress; /share/nasc/AtGenExpress_Developmental_series_shoots_and_stems/ |
| continuous light; soil | CEL/ATGE_4_A.CEL |
| Wt shoot apex, vegetative; 7 days; continuous light; soil | AtGenExpress; /share/nasc/AtGenExpress_Developmental_series_shoots_and_stems/ |
| | CEL/ATGE_6_A.CEL |
| Wt shoot apex, transition (before bolting) 14 days | AtGenExpress; /share/nasc/AtGenExpress_Developmental_series_shoots_and_stems/ |
| continuous light soil | CEL/ATGE_8_A.CEL |
| clv3-7 shoot apex, inflorescence (after bolting) 21+ days | AtGenExpress; /share/nasc/AtGenExpress_Developmental_series_shoots_and_stems/ |
| continuous light soil | CEL/ATGE_46_A.CEL |
| lfy-12 shoot apex, inflorescence (after bolting) 21+ days | AtGenExpress; /share/nasc/AtGenExpress_Developmental_series_shoots_and_stems/ |
| continuous light soil | CEL/ATGE_47_A.CEL |
| ap1-15 shoot apex, inflorescence (after bolting) 21+ days | AtGenExpress; /share/nasc/AtGenExpress_Developmental_series_shoots_and_stems/ |
| continuous light soil | CEL/ATGE_48_A.CEL |
| ap2-6 shoot apex, inflorescence (after bolting) 21+ days | AtGenExpress; /share/nasc/AtGenExpress_Developmental_series_shoots_and_stems/ |
| continuous light soil | CEL/ATGE_49_A.CEL |
| ap3-6 shoot apex, inflorescence (after bolting) 21+ days | AtGenExpress; /share/nasc/AtGenExpress_Developmental_series_shoots_and_stems/ |
| continuous light soil | CEL/ATGE_50_A.CEL |
| ag-12 shoot apex, inflorescence (after bolting) 21+ days | AtGenExpress; /share/nasc/AtGenExpress_Developmental_series_shoots_and_stems/ |
| continuous light soil | CEL/ATGE_51_A.CEL |
| ufo-1 shoot apex, inflorescence (after bolting) 21+ days | AtGenExpress; /share/nasc/AtGenExpress_Developmental_series_shoots_and_stems/ |
| continuous light soil | CEL/ATGE_52_A.CEL |
| Root Quiescent Center; AGL42; quiescent center, | Birnbaum_arexdb; |
| non-specific pattern when roots grow old/long; | |
| QC only and at very early stages | |
| Root Phloem; APL; developing pSE and mSE and CC | Birnbaum_arexdb; |
| between the two: loss of GFP in mature pSE is | |
| accompanied by strong GFP in neighboring CC; | |
| GFP reappears in immature mSE; | |
| Root Cortex; C1 (or 315.1.1) (AT1G09750 promoter); | Birnbaum_arexdb; |
| cortex; from elongation to at least immature root hair dif. | |
| Root Epidermis; GL2; atrichoblasts; from QC and up | Birnbaum_arexdb; |
| Root Ground tissue; J0571; ground: endo + cortex + qc; | Birnbaum_arexdb; |
| from tip all the way up | |
| Lateral Root Cap; J3411; lateral root cap plus epidermis; | Birnbaum_arexdb; |
| from tip to exactly where? at least the differentiation | |
| zone; GFP is about 5X weaker in dif. zone | |
| Root Columella; PET111; columnella - tier 2,3,4; | Birnbaum_arexdb; |
| Root Xylem; S18 (477.1.3) (AT5G12870 promoter); | Birnbaum_arexdb; |
| maturing xylem cells; starts from mid-elongation | |
| and stops half way; GFP switches from pSE to mSE | |
| Root Endodermis; SCR; Marks the QC and the | Birnbaum_arexdb; |
| endodermis at least past the mature hair zone; from QC | |
| all the way up? At least past mature hair zone | |
| Tissue microdissection; Stage I; all radial tissues; | Birnbaum_arexdb; |
| where the root tip reaches its full diameter (about | |
| 0.15 mm from the root tip) | |
| Tissue microdissection; Stage II; all radial tissues; | Birnbaum_arexdb; |
| where cells transition from being optically dense to | |
| a more transparent appearance as they begin | |
| longitudinal expansion | |
| Tissue microdissection; Stage III; all radial tissues; | Birnbaum_arexdb; |
| where root hairs are fully elongated (about 0.45 to 2 mm | |
| from the root tip) | |
| Root Stele; WOL; stele; from QC and starts to fade from | Birnbaum_arexdb; |
| elongation zone | |
| Campb-Col0-Suc | Malcolm_Campbell_1; /share/nasc/Malcolm_Campbell_1/ |
| | CEL/MC001_ATH1_A1- |
| | Campb-317.CEL |
| Campb-Col0-noSuc | Malcolm_Campbell_1; /share/nasc/Malcolm_Campbell_1/ |
| | CEL/MC001_ATH1_A4- |
| | Campb-320.CEL |
| Campb-MYB61-Suc | Malcolm_Campbell_1; /share/nasc/Malcolm_Campbell_1/ |
| | CEL/MC001_ATH1_A7- |
| | Campb-323.CEL |
| Campb-MYB61-noSuc | Malcolm_Campbell_1; /share/nasc/Malcolm_Campbell_1/ |
| | CEL/MC001_ATH1_A10- |
| | Campb-326.CEL |
| Campb-MYB61oe-Suc | Malcolm_Campbell_1; /share/nasc/Malcolm_Campbell_1/ |
| | CEL/MC001_ATH1_A13- |
| | Campb-329(new).CEL |
| Campb-MYB61oe-noSuc | Malcolm_Campbell_1; /share/nasc/Malcolm_Campbell_1/ |
| | CEL/MC001_ATH1_A16- |
| | Campb-332.CEL |
| col; hypocotyl | Malcolm_Campbell_2; /share/nasc/Malcolm_Campbell_2/ |
| | CEL/MC002_ATH1_A10.1- |
| | dubos-wth.CEL |
| max4; hypocotyl | Malcolm_Campbell_2; /share/nasc/Malcolm_Campbell_2/ |
| | CEL/MC002_ATH1_A11.1- |
| | dubos-mxh.CEL |
| axr1; hypocotyl | Malcolm_Campbell_2; /share/nasc/Malcolm_Campbell_2/ |
| | CEL/MC002_ATH1_A12.1- |
| | dubos-arh.CEL |
| col; xylem | Malcolm_Campbell_2; /share/nasc/Malcolm_Campbell_2/ |
| | CEL/MC002_ATH1_A1.1- |
| | dubos-wtx.CEL |
| col; cork | Malcolm_Campbell_2; /share/nasc/Malcolm_Campbell_2/ |
| | CEL/MC002_ATH1_A2.1- |
| | dubos-wtc.CEL |
| myb61 ko; xylem | Malcolm_Campbell_2; /share/nasc/Malcolm_Campbell_2/ |
| | CEL/MC002_ATH1_A3.1- |
| | dubos-6kx.CEL |
| myb61 ko; cork | Malcolm_Campbell_2; /share/nasc/Malcolm_Campbell_2/ |
| | CEL/MC002_ATH1_A4.1- |
| | dubos-6kc.CEL |
| myb50 ko; xylem | Malcolm_Campbell_2; /share/nasc/Malcolm_Campbell_2/ |
| | CEL/MC002_ATH1_A5.1- |
| | dubos-5kx.CEL |
| myb50; cork | Malcolm_Campbell_2; /share/nasc/Malcolm_Campbell_2/ |
| | CEL/MC002_ATH1_A6.1- |
| | dubos-5kc.CEL |
| ler; hypocotyl | Malcolm_Campbell_2; /share/nasc/Malcolm_Campbell_2/ |
| | CEL/MC002_ATH1_A7.1- |
| | dubos-wLh.CEL |
| abi1; hypocotyl (ler background) | Malcolm_Campbell_2; /share/nasc/Malcolm_Campbell_2/ |
| | CEL/MC002_ATH1_A8.1- |
| | dubos-aih.CEL |
| aba1; hypocotyl (ler background) | Malcolm_Campbell_2; /share/nasc/Malcolm_Campbell_2/ |
| | CEL/MC002_ATH1_A9.1- |
| | dubos-aah.CEL |
| seedling; Col0; 10 nM BL for 1 h | AtGenExpress; /share/nasc/AtGen_Brassinolide_timecource_wt_det2_seedlings/ |
| | CEL/RIKENGODA16A.cel |
| seedling; Col0; mock treatment for 3 h | AtGenExpress; /share/nasc/AtGen_Brassinolide_timecource_wt_det2_seedlings/ |
| | CEL/RIKENGODA17AC.cel |
| seedling; Col0; mock treatment for 30 min | AtGenExpress; /share/nasc/AtGen_Brassinolide_timecource_wt_det2_seedlings/ |
| | CEL/RIKENGODA1AC.cel |
| seedling; Col0; 10 nM BL for 3 h | AtGenExpress; /share/nasc/AtGen_Brassinolide_timecource_wt_det2_seedlings/ |
| | CEL/RIKENGODA24A.cel |
| seedling; de-etiolated; mock treatment for 30 min | AtGenExpress; /share/nasc/AtGen_Brassinolide_timecource_wt_det2_seedlings/ |
| | CEL/RIKENGODA31A.cel |
| seedling; de-etiolated; 10 nM BL for 30 min | AtGenExpress; /share/nasc/AtGen_Brassinolide_timecource_wt_det2_seedlings/ |
| | CEL/RIKENGODA32A.cel |
| seedling; de-etiolated; mock treatment for 1 h | AtGenExpress; /share/nasc/AtGen_Brassinolide_timecource_wt_det2_seedlings/ |
| | CEL/RIKENGODA33A.cel |
| seedling; de-etiolated; 10 nM BL for 1 h | AtGenExpress; /share/nasc/AtGen_Brassinolide_timecource_wt_det2_seedlings/ |
| | CEL/RIKENGODA34A.cel |
| seedling; de-etiolated; mock treatment for 3 h | AtGenExpress; /share/nasc/AtGen_Brassinolide_timecource_wt_det2_seedlings/ |
| | CEL/RIKENGODA35A.cel |
| seedling; de-etiolated; 10 nM BL for 3 h | AtGenExpress; /share/nasc/AtGen_Brassinolide_timecource_wt_det2_seedlings/ |
| | CEL/RIKENGODA36A.cel |
| seedling; Col0; 10 nM BL for 30 min | AtGenExpress; /share/nasc/AtGen_Brassinolide_timecource_wt_det2_seedlings/ |
| | CEL/RIKENGODA8A.cel |
| seedling; Col0; mock treatment for 1 h | AtGenExpress; /share/nasc/AtGen_Brassinolide_timecource_wt_det2_seedlings/ |
| | CEL/RIKENGODA9AC.cel |
| complete plant; 10 uM brassinazole 91 (Brz91) | AtGenExpress; /share/nasc/AtGen_effect_brassinosteroid_inhibitors_seeds/ |
| (brassinosteroid biosynthesis inhibitor) for 12 h | CEL/RIKENGODA10A4.cel |
| complete plant; mock treatment for 3 h | AtGenExpress; /share/nasc/AtGen_effect_brassinosteroid_inhibitors_seeds/ |
| | CEL/RIKENGODA1A4.cel |
| complete plant; mock treatment for 3 h | AtGenExpress; /share/nasc/AtGen_effect_brassinosteroid_inhibitors_seeds/ |
| | CEL/RIKENGODA28A4.cel |
| complete plant; mock treatment for 12 h | AtGenExpress; /share/nasc/AtGen_effect_brassinosteroid_inhibitors_seeds/ |
| | CEL/RIKENGODA2A4.cel |
| complete plant; 3 uM brassinazole 220 (Brz220) | AtGenExpress; /share/nasc/AtGen_effect_brassinosteroid_inhibitors_seeds/ |
| (brassinosteroid biosynthesis inhibitor) for 3 h | CEL/RIKENGODA30A4.cel |
| complete plant; 10 uM brassinazole 220 (Brz220) | AtGenExpress; /share/nasc/AtGen_effect_brassinosteroid_inhibitors_seeds/ |
| (brassinosteroid biosynthesis inhibitor) for 3 h | CEL/RIKENGODA7A4.cel |
| complete plant; 10 uM paclobutrazol (GA biosynthesis | AtGenExpress; /share/nasc/AtGen_effect_GA_inhibitors_on_seeds/ |
| inhibitor) for 3 h | CEL/ |
| | RIKENGODA11A2.cel |
| complete plant; 10 uM paclobutrazol (GA biosynthesis | AtGenExpress; /share/nasc/AtGen_effect_GA_inhibitors_on_seeds/ |
| inhibitor) for 12 h | CEL/ |
| | RIKENGODA12A2.cel |
| complete plant; 10 uM prohexadione (GA biosynthesis | AtGenExpress; /share/nasc/AtGen_effect_GA_inhibitors_on_seeds/ |
| inhibitor) for 3 h | CEL/ |
| | RIKENGODA13A2.cel |
| complete plant; 10 uM prohexadione (GA biosynthesis | AtGenExpress; /share/nasc/AtGen_effect_GA_inhibitors_on_seeds/ |
| inhibitor) for 12 h | CEL/ |
| | RIKENGODA14A2.cel |
| complete plant; mock treatment for 3 h | AtGenExpress; /share/nasc/AtGen_effect_GA_inhibitors_on_seeds/ |
| | CEL/ |
| | RIKENGODA1A2.cel |
| complete plant; mock treatment for 12 h | AtGenExpress; /share/nasc/AtGen_effect_GA_inhibitors_on_seeds/ |
| | CEL/ |
| | RIKENGODA2A2.cel |
| complete plant; 10 uM propiconazole (GA biosynthesis | AtGenExpress; /share/nasc/AtGen_effect_GA_inhibitors_on_seeds/ |
| inhibitor) for 3 h | CEL/ |
| | RIKENGODA3A2.cel |
| complete plant; 10 uM propiconazole (GA biosynthesis | AtGenExpress; /share/nasc/AtGen_effect_GA_inhibitors_on_seeds/ |
| inhibitor) for 12 h | CEL/ |
| | RIKENGODA4A2.cel |
| complete plant; 10 uM uniconazole (GA biosynthesis | AtGenExpress; /share/nasc/AtGen_effect_GA_inhibitors_on_seeds/ |
| inhibitor) for 3 h | CEL/ |
| | RIKENGODA5A2.cel |
| complete plant; 10 uM uniconazole (GA biosynthesis | AtGenExpress; /share/nasc/AtGen_effect_GA_inhibitors_on_seeds/ |
| inhibitor) for 12 h | CEL/ |
| | RIKENGODA6A2.cel |
| complete plant; mock treatment for 3 h | AtGenExpress; /share/nasc/AtGen_Effect_of_proteasome_inhibitor_MG13_on_seedlings/ |
| | CEL/RIKENGODA1A9. |
| | cel |
| complete plant; 10 uM MG132 | AtGenExpress; /share/nasc/AtGen_Effect_of_proteasome_inhibitor_MG13_on_seedlings/ |
| (carbobenzoxyl-leucinyl-leucinyl-leucinal) for 3 h | CEL/RIKENGODA22A9. |
| | cel |
| seed; no treatment | AtGenExpress; /share/nasc/AtGen_exp_profiling_early_germinating_seeds/ |
| | CEL/RIKENPRESTON0A.cel |
| seed; imbibed in water (1 hour) | AtGenExpress; /share/nasc/AtGen_exp_profiling_early_germinating_seeds/ |
| | CEL/RIKENPRESTON1A.cel |
| seed; imbibed in water (3 hours) | AtGenExpress; /share/nasc/AtGen_exp_profiling_early_germinating_seeds/ |
| | CEL/RIKENPRESTON2A.cel |
| seedling; 10 uM ABA for 1 h | AtGenExpress; /share/nasc/AtGenExpress_ABA_time_course_in_wildtype_seedlings/ |
| | CEL/RIKENGODA13A. |
| | cel |
| seedling; mock treatment for 3 h | AtGenExpress; /share/nasc/AtGenExpress_ABA_time_course_in_wildtype_seedlings/ |
| | CEL/RIKENGODA17A. |
| | cel |
| seedling; mock treatment for 30 min | AtGenExpress; /share/nasc/AtGenExpress_ABA_time_course_in_wildtype_seedlings/ |
| | CEL/RIKENGODA1A.cel |
| seedling; 10 uM ABA for 3 h | AtGenExpress; /share/nasc/AtGenExpress_ABA_time_course_in_wildtype_seedlings/ |
| | CEL/RIKENGODA21A. |
| | cel |
| seedling; 10 uM ABA for 30 min | AtGenExpress; /share/nasc/AtGenExpress_ABA_time_course_in_wildtype_seedlings/ |
| | CEL/RIKENGODA5A.cel |
| seedling; mock treatment for 1 h | AtGenExpress; /share/nasc/AtGenExpress_ABA_time_course_in_wildtype_seedlings/ |
| | CEL/RIKENGODA9A.cel |
| seedling; 10 uM ACC for 1 h | AtGenExpress; /share/nasc/AtGenExpress_ACC_time_course_in_wildtype_seedlings/ |
| | CEL/RIKENGODA15A. |
| | cel |
| seedling; mock treatment for 3 h | AtGenExpress; /share/nasc/AtGenExpress_ACC_time_course_in_wildtype_seedlings/ |
| | CEL/RIKENGODA17AA. |
| | cel |
| seedling; mock treatment for 30 min | AtGenExpress; /share/nasc/AtGenExpress_ACC_time_course_in_wildtype_seedlings/ |
| | CEL/RIKENGODA1AA. |
| | cel |
| seedling; 10 uM ACC for 3 h | AtGenExpress; /share/nasc/AtGenExpress_ACC_time_course_in_wildtype_seedlings/ |
| | CEL/RIKENGODA23A. |
| | cel |
| seedling; 10 uM ACC for 30 min | AtGenExpress; /share/nasc/AtGenExpress_ACC_time_course_in_wildtype_seedlings/ |
| | CEL/RIKENGODA7A.cel |
| seedling; mock treatment for 1 h | AtGenExpress; /share/nasc/AtGenExpress_ACC_time_course_in_wildtype_seedlings/ |
| | CEL/RIKENGODA9AA. |
| | cel |
| complete plant; 10 uM 2,4,6-T (2,4,6- | AtGenExpress; /share/nasc/AtGenExpress_Effect_of_auxin_inhibitors_on_seedlings/ |
| trihydroxybenzamide) for 3 h | CEL/RIKENGODA23A3. |
| | cel |
| complete plant; 10 uM NPA (naphthylphthalamic | AtGenExpress; /share/nasc/AtGenExpress_Effect_of_auxin_inhibitors_on_seedlings/ |
| acid) (inhibitor of auxin transport) for 3 h | CEL/RIKENGODA26A3. |
| | cel |
| complete plant; 10 uM PCIB (p-chlorophenoxyisobutyric | AtGenExpress; /share/nasc/AtGenExpress_Effect_of_auxin_inhibitors_on_seedlings/ |
| acid) (auxin inhibitor) for 3 h | CEL/RIKENGODA24A3. |
| | cel |
| complete plant; 10 uM TIBA (2,3,5-triiodobenzoic acid) | AtGenExpress; /share/nasc/AtGenExpress_Effect_of_auxin_inhibitors_on_seedlings/ |
| (inhibitor of auxin transport) for 3 h | CEL/RIKENGODA25A3. |
| | cel |
| complete plant; mock treatment for 3 h | AtGenExpress; /share/nasc/AtGenExpress_Effect_of_auxin_inhibitors_on_seedlings/ |
| | CEL/RIKENGODA1A3.cel |
| whole plant; 100 nM castasterone for 3 h | AtGenExpress; /share/nasc/AtGenExpress_Effect_of_brassinosteroids_in_seedlings/ |
| | CEL/RIKENGODA12A6. |
| | cel |
| whole plant; 10 nM brassinolide for 3 h | AtGenExpress; /share/nasc/AtGenExpress_Effect_of_brassinosteroids_in_seedlings/ |
| | CEL/RIKENGODA13A6. |
| | cel |
| whole plant; 10 uM campestanol for 3 h | AtGenExpress; /share/nasc/AtGenExpress_Effect_of_brassinosteroids_in_seedlings/ |
| | CEL/RIKENGODA2A6.cel |
| whole plant; 1 uM 3-dehydro-6-deoxoteasterone for 3 h | AtGenExpress; /share/nasc/AtGenExpress_Effect_of_brassinosteroids_in_seedlings/ |
| | CEL/RIKENGODA7A6.cel |
| whole plant; 1 uM 3-dehydroteasterone for 3 h | AtGenExpress; /share/nasc/AtGenExpress_Effect_of_brassinosteroids_in_seedlings/ |
| | CEL/RIKENGODA8A6.cel |
| whole plant; 1 uM 6-deoxocastasterone for 3 h | AtGenExpress; /share/nasc/AtGenExpress_Effect_of_brassinosteroids_in_seedlings/ |
| | CEL/RIKENGODA11A6. |
| | cel |
| whole plant; 1 uM 6-deoxocathasterone for 3 h | AtGenExpress; /share/nasc/AtGenExpress_Effect_of_brassinosteroids_in_seedlings/ |
| | CEL/RIKENGODA3A6.cel |
| whole plant; 1 uM 6-deoxoteasterone for 3 h | AtGenExpress; /share/nasc/AtGenExpress_Effect_of_brassinosteroids_in_seedlings/ |
| | CEL/RIKENGODA5A6.cel |
| whole plant; 1 uM 6-deoxotyphasterol for 3 h | AtGenExpress; /share/nasc/AtGenExpress_Effect_of_brassinosteroids_in_seedlings/ |
| | CEL/RIKENGODA9A6.cel |
| whole plant; 1 uM cathasterone for 3 h | AtGenExpress; /share/nasc/AtGenExpress_Effect_of_brassinosteroids_in_seedlings/ |
| | CEL/RIKENGODA4A6.cel |
| whole plant; 1 uM teasterone for 3 h | AtGenExpress; /share/nasc/AtGenExpress_Effect_of_brassinosteroids_in_seedlings/ |
| | CEL/RIKENGODA6A6.cel |
| whole plant; 1 uM typhasterol for 3 h | AtGenExpress; /share/nasc/AtGenExpress_Effect_of_brassinosteroids_in_seedlings/ |
| | CEL/RIKENGODA10A6. |
| | cel |
| whole plant; mock treatment for 3 h | AtGenExpress; /share/nasc/AtGenExpress_Effect_of_brassinosteroids_in_seedlings/ |
| | CEL/RIKENGODA1A6.cel |
| complete plant; 10 uM AgNO3 (ethylene inhibitor) | AtGenExpress; /share/nasc/AtGenExpress_Effect_of_ethylene_inhibitors_on_seedlings/ |
| for 3 h | CEL/RIKENGODA19A7. |
| | cel |
| complete plant; mock treatment for 3 h | AtGenExpress; /share/nasc/AtGenExpress_Effect_of_ethylene_inhibitors_on_seedlings/ |
| | CEL/RIKENGODA1A7. |
| | cel |
| complete plant; 10 uM AVG (aminoethoxyvinylglycine) | AtGenExpress; /share/nasc/AtGenExpress_Effect_of_ethylene_inhibitors_on_seedlings/ |
| (ethylene inhibitor) for 3 h | CEL/RIKENGODA20A7. |
| | cel |
| seedling; 1 uM IAA for 1 h | AtGenExpress; /share/nasc/AtGenExpress_IAA_time_course_in_wildtype_seedlings/ |
| | CEL/RIKENGODA10A.cel |
| seedling; mock treatment for 3 h | AtGenExpress; /share/nasc/AtGenExpress_IAA_time_course_in_wildtype_seedlings/ |
| | CEL/RIKENGODA17AD. |
| | cel |
| seedling; 1 uM IAA for 3 h | AtGenExpress; /share/nasc/AtGenExpress_IAA_time_course_in_wildtype_seedlings/ |
| | CEL/RIKENGODA18A.cel |
| seedling; mock treatment for 30 min | AtGenExpress; /share/nasc/AtGenExpress_IAA_time_course_in_wildtype_seedlings/ |
| | CEL/RIKENGODA1AD.cel |
| seedling; 1 uM IAA for 30 min | AtGenExpress; /share/nasc/AtGenExpress_IAA_time_course_in_wildtype_seedlings/ |
| | CEL/RIKENGODA2A.cel |
| seedling; mock treatment for 1 h | AtGenExpress; /share/nasc/AtGenExpress_IAA_time_course_in_wildtype_seedlings/ |
| | CEL/RIKENGODA9AD.cel |
| seedling; 10 uM MJ for 1 h | AtGenExpress; /share/nasc/AtGenExpress_Methyl_Jasmonate_time_course_in_wildtype/ |
| | CEL/RIKENGODA14A. |
| | cel |
| seedling; mock treatment for 3 h | AtGenExpress; /share/nasc/AtGenExpress_Methyl_Jasmonate_time_course_in_wildtype/ |
| | CEL/RIKENGODA17AF. |
| | cel |
| seedling; mock treatment for 30 min | AtGenExpress; /share/nasc/AtGenExpress_Methyl_Jasmonate_time_course_in_wildtype/ |
| | CEL/RIKENGODA1AF. |
| | cel |
| seedling; 10 uM MJ for 3 h | AtGenExpress; /share/nasc/AtGenExpress_Methyl_Jasmonate_time_course_in_wildtype/ |
| | CEL/RIKENGODA22A. |
| | cel |
| seedling; 10 uM MJ for 30 min | AtGenExpress; /share/nasc/AtGenExpress_Methyl_Jasmonate_time_course_in_wildtype/ |
| | CEL/RIKENGODA6A. |
| | cel |
| seedling; mock treatment for 1 h | AtGenExpress; /share/nasc/AtGenExpress_Methyl_Jasmonate_time_course_in_wildtype/ |
| | CEL/RIKENGODA9AF. |
| | cel |
| seedling; 1 uM zeatin for 1 h | AtGenExpress; /share/nasc/AtGenExpress_Zeatin_time_course_in_wildtype_seedlings/ |
| | CEL/RIKENGODA11A. |
| | cel |
| seedling; 1 uM zeatin for 30 min | AtGenExpress; /share/nasc/AtGenExpress_Zeatin_time_course_in_wildtype_seedlings/ |
| | CEL/RIKENGODA3A. |
| | cel |
| seedling; 1 uM zeatin for 3 h | AtGenExpress; /share/nasc/AtGenExpress_Zeatin_time_course_in_wildtype_seedlings/ |
| | CEL/RIKENGODA19A. |
| | cel |
| seedling; mock treatment for 1 h | AtGenExpress; /share/nasc/AtGenExpress_Zeatin_time_course_in_wildtype_seedlings/ |
| | CEL/RIKENGODA9AG. |
| | cel |
| seedling; mock treatment for 30 min | AtGenExpress; /share/nasc/AtGenExpress_Zeatin_time_course_in_wildtype_seedlings/ |
| | CEL/RIKENGODA1AG. |
| | cel |
| seedling; mock treatment for 3 h | AtGenExpress; /share/nasc/AtGenExpress_Zeatin_time_course_in_wildtype_seedlings/ |
| | CEL/RIKENGODA17AG. |
| | cel |
| seeds; water treatment for 3 hours | AtGenExpress; /share/nasc/AtGenExpress_Basic_hormone_treatment_of_seeds/ |
| | CEL/RIKENLI1A.cel |
| seeds; water treatment for 6 hours | AtGenExpress; /share/nasc/AtGenExpress_Basic_hormone_treatment_of_seeds/ |
| | CEL/RIKENLI2A.cel |
| seeds; water treatment for 9 hours | AtGenExpress; /share/nasc/AtGenExpress_Basic_hormone_treatment_of_seeds/ |
| | CEL/RIKENLI3A.cel |
| seeds; 5 uM gibberellin treatment for 3 hours | AtGenExpress; /share/nasc/AtGenExpress_Basic_hormone_treatment_of_seeds/ |
| | CEL/RIKENLI4A.cel |
| seeds; 5 uM gibberellin treatment for 6 hours | AtGenExpress; /share/nasc/AtGenExpress_Basic_hormone_treatment_of_seeds/ |
| | CEL/RIKENLI5A.cel |
| seeds; 5 uM gibberellin treatment for 9 hours | AtGenExpress; /share/nasc/AtGenExpress_Basic_hormone_treatment_of_seeds/ |
| | CEL/RIKENLI6A.cel |
| Col0; complete plant; No treatment | AtGenExpress; /share/nasc/AtGenExpress_Cytokinin_treatment_of_seedlings/ |
| | CEL/NO10.cel |
| Col0; complete plant; 20 uM t-zeatin for 3 h | AtGenExpress; /share/nasc/AtGenExpress_Cytokinin_treatment_of_seedlings/ |
| | CEL/NO192.cel |
| ARR22-ox; complete plant; No treatment | AtGenExpress; /share/nasc/AtGenExpress_Cytokinin_treatment_of_seedlings/ |
| | CEL/NO28.cel |
| ARR22-ox; complete plant; 20 uM t-zeatin for 3 h | AtGenExpress; /share/nasc/AtGenExpress_Cytokinin_treatment_of_seedlings/ |
| | CEL/NO31.cel |
| seed; 24 h imbibed in 3 uM ABA | AtGenExpress; /share/nasc/AtGenExpress_Effect_of_ABA_during_seed_imbibition/ |
| | CEL/RIKENNAKABAYASHI3A. |
| | cel |
| seed; 24 h imbibed in 30 uM ABA | AtGenExpress; /share/nasc/AtGenExpress_Effect_of_ABA_during_seed_imbibition/ |
| | CEL/RIKENNAKABAYASHI4A. |
| | cel |
| seed; 24 h imbibed in water | AtGenExpress; /share/nasc/AtGenExpress_Effect_of_ABA_during_seed_imbibition/ |
| | CEL/RIKENNAKABAYASHI2A. |
| | cel |
| seed; no treatment | AtGenExpress; /share/nasc/AtGenExpress_Effect_of_ABA_during_seed_imbibition/ |
| | CEL/RIKENNAKABAYASHI1A. |
| | cel |
| Controlstress-Shoots-0 h | AtGenExpress; /share/nasc/AtGenExpress_Stress_Treatments_Control_plants/ |
| | CEL/0011.CEL |
| Controlstress-Roots-0 h | AtGenExpress; /share/nasc/AtGenExpress_Stress_Treatments_Control_plants/ |
| | CEL/0021.CEL |
| Controlstress-Shoots-0.5 h | AtGenExpress; /share/nasc/AtGenExpress_Stress_Treatments_Control_plants/ |
| | CEL/0111.CEL |
| Controlstress-Roots-0.5 h | AtGenExpress; /share/nasc/AtGenExpress_Stress_Treatments_Control_plants/ |
| | CEL/0121.CEL |
| Controlstress-Shoots-1.0 h | AtGenExpress; /share/nasc/AtGenExpress_Stress_Treatments_Control_plants/ |
| | CEL/0211.CEL |
| Controlstress-Roots-1.0 h | AtGenExpress; /share/nasc/AtGenExpress_Stress_Treatments_Control_plants/ |
| | CEL/0221.CEL |
| Controlstress-Shoots-3.0 h | AtGenExpress; /share/nasc/AtGenExpress_Stress_Treatments_Control_plants/ |
| | CEL/0311.CEL |
| Controlstress-Roots-3.0 h | AtGenExpress; /share/nasc/AtGenExpress_Stress_Treatments_Control_plants/ |
| | CEL/0321.CEL |
| Controlstress-Shoots-6.0 h | AtGenExpress; /share/nasc/AtGenExpress_Stress_Treatments_Control_plants/ |
| | CEL/0411.CEL |
| Controlstress-Roots-6.0 h | AtGenExpress; /share/nasc/AtGenExpress_Stress_Treatments_Control_plants/ |
| | CEL/0421.CEL |
| Controlstress-Shoots-12.0 h | AtGenExpress; /share/nasc/AtGenExpress_Stress_Treatments_Control_plants/ |
| | CEL/0511.CEL |
| Controlstress-Roots-12.0 h | AtGenExpress; /share/nasc/AtGenExpress_Stress_Treatments_Control_plants/ |
| | CEL/0521.CEL |
| Controlstress-Shoots-24.0 h | AtGenExpress; /share/nasc/AtGenExpress_Stress_Treatments_Control_plants/ |
| | CEL/0611.CEL |
| Controlstress-Roots-24.0 h | AtGenExpress; /share/nasc/AtGenExpress_Stress_Treatments_Control_plants/ |
| | CEL/0621.CEL |
| Controlstress-Shoots-0.25 h | AtGenExpress; /share/nasc/AtGenExpress_Stress_Treatments_Control_plants/ |
| | CEL/0711.CEL |
| Controlstress-Roots-0.25 h | AtGenExpress; /share/nasc/AtGenExpress_Stress_Treatments_Control_plants/ |
| | CEL/0721.CEL |
| Controlstress-Shoots-4.0 h | AtGenExpress; /share/nasc/AtGenExpress_Stress_Treatments_Control_plants/ |
| | CEL/0811.CEL |
| Controlstress-Roots-4.0 h | AtGenExpress; /share/nasc/AtGenExpress_Stress_Treatments_Control_plants/ |
| | CEL/0821.CEL |
| Coldstress(4?C)-Shoots-0.5 h | AtGenExpress; /share/nasc/AtGenExpress_Stress_Treatments_Cold_stress/ |
| | CEL/1111.CEL |
| Coldstress(4?C)-Roots-0.5 h | AtGenExpress; /share/nasc/AtGenExpress_Stress_Treatments_Cold_stress/ |
| | CEL/1121.CEL |
| Coldstress(4?C)-Shoots-1.0 h | AtGenExpress; /share/nasc/AtGenExpress_Stress_Treatments_Cold_stress/ |
| | CEL/1211.CEL |
| Coldstress(4?C)-Roots-1.0 h | AtGenExpress; /share/nasc/AtGenExpress_Stress_Treatments_Cold_stress/ |
| | CEL/1221.CEL |
| Coldstress(4?C)-Shoots-3.0 h | AtGenExpress; /share/nasc/AtGenExpress_Stress_Treatments_Cold_stress/ |
| | CEL/1311.CEL |
| Coldstress(4?C)-Roots-3.0 h | AtGenExpress; /share/nasc/AtGenExpress_Stress_Treatments_Cold_stress/ |
| | CEL/1321.CEL |
| Coldstress(4?C)-Shoots-6.0 h | AtGenExpress; /share/nasc/AtGenExpress_Stress_Treatments_Cold_stress/ |
| | CEL/1411.CEL |
| Coldstress(4?C)-Roots-6.0 h | AtGenExpress; /share/nasc/AtGenExpress_Stress_Treatments_Cold_stress/ |
| | CEL/1421.CEL |
| Coldstress(4?C)-Shoots-12.0 h | AtGenExpress; /share/nasc/AtGenExpress_Stress_Treatments_Cold_stress/ |
| | CEL/1511.CEL |
| Coldstress(4?C)-Roots-12.0 h | AtGenExpress; /share/nasc/AtGenExpress_Stress_Treatments_Cold_stress/ |
| | CEL/1521.CEL |
| Coldstress(4?C)-Shoots-24.0 h | AtGenExpress; /share/nasc/AtGenExpress_Stress_Treatments_Cold_stress/ |
| | CEL/1611.CEL |
| Coldstress(4?C)-Roots-24.0 h | AtGenExpress; /share/nasc/AtGenExpress_Stress_Treatments_Cold_stress/ |
| | CEL/1621.CEL |
| Osmoticstress-Shoots-0.5 h | AtGenExpress; /share/nasc/AtGenExpress_Stress_Treatments_Osmotic_stress/ |
| | CEL/2111.CEL |
| Osmoticstress-Roots-0.5 h | AtGenExpress; /share/nasc/AtGenExpress_Stress_Treatments_Osmotic_stress/ |
| | CEL/2121.CEL |
| Osmoticstress-Shoots-1.0 h | AtGenExpress; /share/nasc/AtGenExpress_Stress_Treatments_Osmotic_stress/ |
| | CEL/2211.CEL |
| Osmoticstress-Roots-1.0 h | AtGenExpress; /share/nasc/AtGenExpress_Stress_Treatments_Osmotic_stress/ |
| | CEL/2221.CEL |
| Osmoticstress-Shoots-3.0 h | AtGenExpress; /share/nasc/AtGenExpress_Stress_Treatments_Osmotic_stress/ |
| | CEL/2311.CEL |
| Osmoticstress-Roots-3.0 h | AtGenExpress; /share/nasc/AtGenExpress_Stress_Treatments_Osmotic_stress/ |
| | CEL/2321.CEL |
| Osmoticstress-Shoots-6.0 h | AtGenExpress; /share/nasc/AtGenExpress_Stress_Treatments_Osmotic_stress/ |
| | CEL/2411.CEL |
| Osmoticstress-Roots-6.0 h | AtGenExpress; /share/nasc/AtGenExpress_Stress_Treatments_Osmotic_stress/ |
| | CEL/2421.CEL |
| Osmoticstress-Shoots-12.0 h | AtGenExpress; /share/nasc/AtGenExpress_Stress_Treatments_Osmotic_stress/ |
| | CEL/2511.CEL |
| Osmoticstress-Roots-12.0 h | AtGenExpress; /share/nasc/AtGenExpress_Stress_Treatments_Osmotic_stress/ |
| | CEL/2521.CEL |
| Osmoticstress-Shoots-24.0 h | AtGenExpress; /share/nasc/AtGenExpress_Stress_Treatments_Osmotic_stress/ |
| | CEL/2611.CEL |
| Osmoticstress-Roots-24.0 h | AtGenExpress; /share/nasc/AtGenExpress_Stress_Treatments_Osmotic_stress/ |
| | CEL/2621.CEL |
| Saltstress-Shoots-0.5 h | AtGenExpress; /share/nasc/AtGenExpress_Stress_Treatments_Salt_stress/ |
| | CEL/3111.CEL |
| Saltstress-Roots-0.5 h | AtGenExpress; /share/nasc/AtGenExpress_Stress_Treatments_Salt_stress/ |
| | CEL/3121.CEL |
| Saltstress-Shoots-1.0 h | AtGenExpress; /share/nasc/AtGenExpress_Stress_Treatments_Salt_stress/ |
| | CEL/3211.CEL |
| Saltstress-Roots-1.0 h | AtGenExpress; /share/nasc/AtGenExpress_Stress_Treatments_Salt_stress/ |
| | CEL/3221.CEL |
| Saltstress-Shoots-3.0 h | AtGenExpress; /share/nasc/AtGenExpress_Stress_Treatments_Salt_stress/ |
| | CEL/3311.CEL |
| Saltstress-Roots-3.0 h | AtGenExpress; /share/nasc/AtGenExpress_Stress_Treatments_Salt_stress/ |
| | CEL/3321.CEL |
| Saltstress-Shoots-6.0 h | AtGenExpress; /share/nasc/AtGenExpress_Stress_Treatments_Salt_stress/ |
| | CEL/3411.CEL |
| Saltstress-Roots-6.0 h | AtGenExpress; /share/nasc/AtGenExpress_Stress_Treatments_Salt_stress/ |
| | CEL/3421.CEL |
| Saltstress-Shoots-12.0 h | AtGenExpress; /share/nasc/AtGenExpress_Stress_Treatments_Salt_stress/ |
| | CEL/3511.CEL |
| Saltstress-Roots-12.0 h | AtGenExpress; /share/nasc/AtGenExpress_Stress_Treatments_Salt_stress/ |
| | CEL/3521.CEL |
| Saltstress-Shoots-24.0 h | AtGenExpress; /share/nasc/AtGenExpress_Stress_Treatments_Salt_stress/ |
| | CEL/3611.CEL |
| Saltstress-Roots-24.0 h | AtGenExpress; /share/nasc/AtGenExpress_Stress_Treatments_Salt_stress/ |
| | CEL/3621.CEL |
| Droughtstress-Shoots-0.5 h | AtGenExpress; /share/nasc/AtGenExpress_Stress_Treatments_Drought_stress/ |
| | CEL/4111.CEL |
| Droughtstress-Roots-0.5 h | AtGenExpress; /share/nasc/AtGenExpress_Stress_Treatments_Drought_stress/ |
| | CEL/4121.CEL |
| Droughtstress-Shoots-1.0 h | AtGenExpress; /share/nasc/AtGenExpress_Stress_Treatments_Drought_stress/ |
| | CEL/4211.CEL |
| Droughtstress-Roots-1.0 h | AtGenExpress; /share/nasc/AtGenExpress_Stress_Treatments_Drought_stress/ |
| | CEL/4221.CEL |
| Droughtstress-Shoots-3.0 h | AtGenExpress; /share/nasc/AtGenExpress_Stress_Treatments_Drought_stress/ |
| | CEL/4311.CEL |
| Droughtstress-Roots-3.0 h | AtGenExpress; /share/nasc/AtGenExpress_Stress_Treatments_Drought_stress/ |
| | CEL/4321.CEL |
| Droughtstress-Shoots-6.0 h | AtGenExpress; /share/nasc/AtGenExpress_Stress_Treatments_Drought_stress/ |
| | CEL/4411.CEL |
| Droughtstress-Roots-6.0 h | AtGenExpress; /share/nasc/AtGenExpress_Stress_Treatments_Drought_stress/ |
| | CEL/4421.CEL |
| Droughtstress-Shoots-12.0 h | AtGenExpress; /share/nasc/AtGenExpress_Stress_Treatments_Drought_stress/ |
| | CEL/4511.CEL |
| Droughtstress-Roots-12.0 h | AtGenExpress; /share/nasc/AtGenExpress_Stress_Treatments_Drought_stress/ |
| | CEL/4521.CEL |
| Droughtstress-Shoots-24.0 h | AtGenExpress; /share/nasc/AtGenExpress_Stress_Treatments_Drought_stress/ |
| | CEL/4611.CEL |
| Droughtstress-Roots-24.0 h | AtGenExpress; /share/nasc/AtGenExpress_Stress_Treatments_Drought_stress/ |
| | CEL/4621.CEL |
| Droughtstress-Shoots-0.25 h | AtGenExpress; /share/nasc/AtGenExpress_Stress_Treatments_Drought_stress/ |
| | CEL/4711.CEL |
| Droughtstress-Roots-0.25 h | AtGenExpress; /share/nasc/AtGenExpress_Stress_Treatments_Drought_stress/ |
| | CEL/4721.CEL |
| Genotoxicstress-Shoots-0.5 h | AtGenExpress; /share/nasc/AtGenExpress_Stress_Treatments_Genotoxic_stress/ |
| | CEL/5111.CEL |
| Genotoxicstress-Roots-0.5 h | AtGenExpress; /share/nasc/AtGenExpress_Stress_Treatments_Genotoxic_stress/ |
| | CEL/5121.CEL |
| Genotoxicstress-Shoots-1.0 h | AtGenExpress; /share/nasc/AtGenExpress_Stress_Treatments_Genotoxic_stress/ |
| | CEL/5211.CEL |
| Genotoxicstress-Roots-1.0 h | AtGenExpress; /share/nasc/AtGenExpress_Stress_Treatments_Genotoxic_stress/ |
| | CEL/5221.CEL |
| Genotoxicstress-Shoots-3.0 h | AtGenExpress; /share/nasc/AtGenExpress_Stress_Treatments_Genotoxic_stress/ |
| | CEL/5311.CEL |
| Genotoxicstress-Roots-3.0 h | AtGenExpress; /share/nasc/AtGenExpress_Stress_Treatments_Genotoxic_stress/ |
| | CEL/5321.CEL |
| Genotoxicstress-Shoots-6.0 h | AtGenExpress; /share/nasc/AtGenExpress_Stress_Treatments_Genotoxic_stress/ |
| | CEL/5411.CEL |
| Genotoxicstress-Roots-6.0 h | AtGenExpress; /share/nasc/AtGenExpress_Stress_Treatments_Genotoxic_stress/ |
| | CEL/5421.CEL |
| Genotoxicstress-Shoots-12.0 h | AtGenExpress; /share/nasc/AtGenExpress_Stress_Treatments_Genotoxic_stress/ |
| | CEL/5511.CEL |
| Genotoxicstress-Roots-12.0 h | AtGenExpress; /share/nasc/AtGenExpress_Stress_Treatments_Genotoxic_stress/ |
| | CEL/5521.CEL |
| Genotoxicstress-Shoots-24.0 h | AtGenExpress; /share/nasc/AtGenExpress_Stress_Treatments_Genotoxic_stress/ |
| | CEL/5611.CEL |
| Genotoxicstress-Roots-24.0 h | AtGenExpress; /share/nasc/AtGenExpress_Stress_Treatments_Genotoxic_stress/ |
| | CEL/5621.CEL |
| Oxidativestress-Shoots-0.5 h | AtGenExpress; /share/nasc/AtGenExpress_Stress_Treatments_Oxidative_stress/ |
| | CEL/6111.CEL |
| Oxidativestress-Roots-0.5 h | AtGenExpress; /share/nasc/AtGenExpress_Stress_Treatments_Oxidative_stress/ |
| | CEL/6122.CEL |
| Oxidativestress-Shoots-1.0 h | AtGenExpress; /share/nasc/AtGenExpress_Stress_Treatments_Oxidative_stress/ |
| | CEL/6211.CEL |
| Oxidativestress-Roots-1.0 h | AtGenExpress; /share/nasc/AtGenExpress_Stress_Treatments_Oxidative_stress/ |
| | CEL/6223.CEL |
| Oxidativestress-Shoots-3.0 h | AtGenExpress; /share/nasc/AtGenExpress_Stress_Treatments_Oxidative_stress/ |
| | CEL/6311.CEL |
| Oxidativestress-Roots-3.0 h | AtGenExpress; /share/nasc/AtGenExpress_Stress_Treatments_Oxidative_stress/ |
| | CEL/6322.CEL |
| Oxidativestress-Shoots-6.0 h | AtGenExpress; /share/nasc/AtGenExpress_Stress_Treatments_Oxidative_stress/ |
| | CEL/6411.CEL |
| Oxidativestress-Roots-6.0 h | AtGenExpress; /share/nasc/AtGenExpress_Stress_Treatments_Oxidative_stress/ |
| | CEL/6421.CEL |
| Oxidativestress-Shoots-12.0 h | AtGenExpress; /share/nasc/AtGenExpress_Stress_Treatments_Oxidative_stress/ |
| | CEL/6511.CEL |
| Oxidativestress-Roots-12.0 h | AtGenExpress; /share/nasc/AtGenExpress_Stress_Treatments_Oxidative_stress/ |
| | CEL/6523.CEL |
| Oxidativestress-Shoots-24.0 h | AtGenExpress; /share/nasc/AtGenExpress_Stress_Treatments_Oxidative_stress/ |
| | CEL/6611.CEL |
| Oxidativestress-Roots-24.0 h | AtGenExpress; /share/nasc/AtGenExpress_Stress_Treatments_Oxidative_stress/ |
| | CEL/6621.CEL |
| UV-Bstress-Shoots-0.5 h | AtGenExpress; /share/nasc/AtGenExpress_Stress_Treatments_UV- |
| | B_stress/CEL/7111.CEL |
| UV-Bstress-Roots-0.5 h | AtGenExpress; /share/nasc/AtGenExpress_Stress_Treatments_UV- |
| | B_stress/CEL/7121.CEL |
| UV-Bstress-Shoots-1.0 h | AtGenExpress; /share/nasc/AtGenExpress_Stress_Treatments_UV- |
| | B_stress/CEL/7211.CEL |
| UV-Bstress-Roots-1.0 h | AtGenExpress; /share/nasc/AtGenExpress_Stress_Treatments_UV- |
| | B_stress/CEL/7221.CEL |
| UV-Bstress-Shoots-3.0 h | AtGenExpress; /share/nasc/AtGenExpress_Stress_Treatments_UV- |
| | B_stress/CEL/7311.CEL |
| UV-Bstress-Roots-3.0 h | AtGenExpress; /share/nasc/AtGenExpress_Stress_Treatments_UV- |
| | B_stress/CEL/7321.CEL |
| UV-Bstress-Shoots-6.0 h | AtGenExpress; /share/nasc/AtGenExpress_Stress_Treatments_UV- |
| | B_stress/CEL/7411.CEL |
| UV-Bstress-Roots-6.0 h | AtGenExpress; /share/nasc/AtGenExpress_Stress_Treatments_UV- |
| | B_stress/CEL/7421.CEL |
| UV-Bstress-Shoots-12.0 h | AtGenExpress; /share/nasc/AtGenExpress_Stress_Treatments_UV- |
| | B_stress/CEL/7511.CEL |
| UV-Bstress-Roots-12.0 h | AtGenExpress; /share/nasc/AtGenExpress_Stress_Treatments_UV- |
| | B_stress/CEL/7521.CEL |
| UV-Bstress-Shoots-24.0 h | AtGenExpress; /share/nasc/AtGenExpress_Stress_Treatments_UV- |
| | B_stress/CEL/7611.CEL |
| UV-Bstress-Roots-24.0 h | AtGenExpress; /share/nasc/AtGenExpress_Stress_Treatments_UV- |
| | B_stress/CEL/7621.CEL |
| UV-Bstress-Shoots-0.25 h | AtGenExpress; /share/nasc/AtGenExpress_Stress_Treatments_UV- |
| | B_stress/CEL/7711.CEL |
| UV-Bstress-Roots-0.25 h | AtGenExpress; /share/nasc/AtGenExpress_Stress_Treatments_UV- |
| | B_stress/CEL/7721.CEL |
| Woundingstress-Shoots-0.5 h | AtGenExpress; /share/nasc/AtGenExpress_Stress_Treatments_Wounding_stress/ |
| | CEL/8111.CEL |
| Woundingstress-Roots-0.5 h | AtGenExpress; /share/nasc/AtGenExpress_Stress_Treatments_Wounding_stress/ |
| | CEL/8124.CEL |
| Woundingstress-Shoots-1.0 h | AtGenExpress; /share/nasc/AtGenExpress_Stress_Treatments_Wounding_stress/ |
| | CEL/8211.CEL |
| Woundingstress-Roots-1.0 h | AtGenExpress; /share/nasc/AtGenExpress_Stress_Treatments_Wounding_stress/ |
| | CEL/8224.CEL |
| Woundingstress-Shoots-3.0 h | AtGenExpress; /share/nasc/AtGenExpress_Stress_Treatments_Wounding_stress/ |
| | CEL/8313.CEL |
| Woundingstress-Roots-3.0 h | AtGenExpress; /share/nasc/AtGenExpress_Stress_Treatments_Wounding_stress/ |
| | CEL/8324.CEL |
| Woundingstress-Shoots-6.0 h | AtGenExpress; /share/nasc/AtGenExpress_Stress_Treatments_Wounding_stress/ |
| | CEL/8411.CEL |
| Woundingstress-Roots-6.0 h | AtGenExpress; /share/nasc/AtGenExpress_Stress_Treatments_Wounding_stress/ |
| | CEL/8423.CEL |
| Woundingstress-Shoots-12.0 h | AtGenExpress; /share/nasc/AtGenExpress_Stress_Treatments_Wounding_stress/ |
| | CEL/8511.CEL |
| Woundingstress-Roots-12.0 h | AtGenExpress; /share/nasc/AtGenExpress_Stress_Treatments_Wounding_stress/ |
| | CEL/8524.CEL |
| Woundingstress-Shoots-24.0 h | AtGenExpress; /share/nasc/AtGenExpress_Stress_Treatments_Wounding_stress/ |
| | CEL/8611.CEL |
| Woundingstress-Roots-24.0 h | AtGenExpress; /share/nasc/AtGenExpress_Stress_Treatments_Wounding_stress/ |
| | CEL/8621.CEL |
| Woundingstress-Shoots-0.25 h | AtGenExpress; /share/nasc/AtGenExpress_Stress_Treatments_Wounding_stress/ |
| | CEL/8712.CEL |
| Woundingstress-Roots-0.25 h | AtGenExpress; /share/nasc/AtGenExpress_Stress_Treatments_Wounding_stress/ |
| | CEL/8723.CEL |
| Heatstress-Shoots-0.5 h | AtGenExpress; /share/nasc/AtGenExpress_Stress_Treatments_Heat_stress/ |
| | CEL/9111.CEL |
| Heatstress-Roots-0.5 h | AtGenExpress; /share/nasc/AtGenExpress_Stress_Treatments_Heat_stress/ |
| | CEL/9121.CEL |
| Heatstress-Shoots-1.0 h | AtGenExpress; /share/nasc/AtGenExpress_Stress_Treatments_Heat_stress/ |
| | CEL/9211.CEL |
| Heatstress-Roots-1.0 h | AtGenExpress; /share/nasc/AtGenExpress_Stress_Treatments_Heat_stress/ |
| | CEL/9221.CEL |
| Heatstress-Shoots-3.0 h | AtGenExpress; /share/nasc/AtGenExpress_Stress_Treatments_Heat_stress/ |
| | CEL/9311.CEL |
| Heatstress-Roots-3.0 h | AtGenExpress; /share/nasc/AtGenExpress_Stress_Treatments_Heat_stress/ |
| | CEL/9321.CEL |
| Heatstress (3 h) + 3 h recovery-Shoots-6.0 h | AtGenExpress; /share/nasc/AtGenExpress_Stress_Treatments_Heat_stress/ |
| | CEL/9411.CEL |
| Heatstress (3 h) + 3 h recovery-Roots-6.0 h | AtGenExpress; /share/nasc/AtGenExpress_Stress_Treatments_Heat_stress/ |
| | CEL/9421.CEL |
| Heatstress (3 h) + 9 h recovery-Shoots-12.0 h | AtGenExpress; /share/nasc/AtGenExpress_Stress_Treatments_Heat_stress/ |
| | CEL/9511.CEL |
| Heatstress (3 h) + 9 h recovery-Roots-12.0 h | AtGenExpress; /share/nasc/AtGenExpress_Stress_Treatments_Heat_stress/ |
| | CEL/9521.CEL |
| Heatstress (3 h) + 21 h recovery-Shoots-24.0 h | AtGenExpress; /share/nasc/AtGenExpress_Stress_Treatments_Heat_stress/ |
| | CEL/9611.CEL |
| Heatstress (3 h) + 21 h recovery-Roots-24.0 h | AtGenExpress; /share/nasc/AtGenExpress_Stress_Treatments_Heat_stress/ |
| | CEL/9621.CEL |
| Heatstress-Shoots-0.25 h | AtGenExpress; /share/nasc/AtGenExpress_Stress_Treatments_Heat_stress/ |
| | CEL/9711.CEL |
| Heatstress-Roots-0.25 h | AtGenExpress; /share/nasc/AtGenExpress_Stress_Treatments_Heat_stress/ |
| | CEL/9721.CEL |
| Heatstress (3 h) + 1 h recovery-Shoots-4.0 h | AtGenExpress; /share/nasc/AtGenExpress_Stress_Treatments_Heat_stress/ |
| | CEL/9811.CEL |
| Heatstress (3 h) + 1 h recovery-Roots-4.0 h | AtGenExpress; /share/nasc/AtGenExpress_Stress_Treatments_Heat_stress/ |
| | CEL/9821.CEL |
| lateral root initiation study in xylem pole pericycle cells; | in-house experiment |
| ivsme_Col-0_NPA | |
| lateral root initiation study in xylem pole pericycle cells; | in-house experiment |
| ivsme_Col-0_2hNAA | |
| lateral root initiation study in xylem pole pericycle cells; | in-house experiment |
| ivsme_Col-0_6hNAA | |
| overexpression of two prohibitins in seedlings; | in-house experiment |
| olake_AtPHB3_overexp | |
| overexpression of two prohibitins in seedlings; | in-house experiment |
| olake_AtPHB4_overexp | |
| overexpression of two prohibitins in seedlings; | in-house experiment |
| olake_overexp_control | |
| comparison of 3 mutants (2 prohibitin, 1 auxin | in-house experiment |
| glucosyltransferase) to wild-type in seedlings; | |
| olake_knockout_AtPHB3 | |
| comparison of 3 mutants (2 prohibitin, 1 auxin | in-house experiment |
| glucosyltransferase) to wild-type in seedlings; | |
| olake_knockout_AtPHB4 | |
| comparison of 3 mutants (2 prohibitin, 1 auxin | in-house experiment |
| glucosyltransferase) to wild-type in seedlings; | |
| olake_control | |
| comparison of 3 mutants (2 prohibitin, 1 auxin | in-house experiment |
| glucosyltransferase) to wild-type in seedlings; | |
| olake_overexpression_UGTx | |
| WT landsberg erecta shoot apex seedlings compared to | in-house experiment |
| narrow leaf mutants elongata 2, 3, 4, angusta 4, | |
| drl1-2; defle_Ler | |
| WT landsberg erecta shoot apex seedlings compared to | in-house experiment |
| narrow leaf mutants elongata 2, 3, 4, angusta 4, | |
| drl1-2; defle_elo2 | |
| WT landsberg erecta shoot apex seedlings compared to | in-house experiment |
| narrow leaf mutants elongata 2, 3, 4, angusta 4, | |
| drl1-2; defle_drl1-2 | |
| WT landsberg erecta shoot apex seedlings compared to | in-house experiment |
| narrow leaf mutants elongata 2, 3, 4, angusta 4, | |
| drl1-2; defle_ang4 | |
| WT landsberg erecta shoot apex seedlings compared to | in-house experiment |
| narrow leaf mutants elongata 2, 3, 4, angusta 4, | |
| drl1-2; defle_elo3 | |
| WT shoot apex seedlings compared to narrow leaf | in-house experiment |
| mutants ELP3 (2) and SWP1 (1); defle_Ws | |
| WT shoot apex seedlings compared to narrow leaf | in-house experiment |
| mutants ELP3 (2) and SWP1 (1); defle_N3983 | |
| WT shoot apex seedlings compared to narrow leaf | in-house experiment |
| mutants ELP3 (2) and SWP1 (1); defle_219E08 | |
| WT shoot apex seedlings compared to narrow leaf | in-house experiment |
| mutants ELP3 (2) and SWP1 (1); defle_Swp1 | |
| WT landsberg erecta leaves 6 compared to broad | in-house experiment |
| leaf mutant rotunda 2; defle_ron2 | |
| WT landsberg erecta leaves 6 compared to broad | in-house experiment |
| leaf mutant rotunda 2; defle_Ler | |
| WT landsberg erecta compared to broad leaf mutant | in-house experiment |
| rotunda 1; defle_Ler | |
| WT landsberg erecta compared to broad leaf mutant | in-house experiment |
| rotunda 1; defle_ron1 | |
| WT leaves profiling at different stages of development | in-house experiment |
| (9, 15, 22 days); gebee_15 | |
| WT leaves profiling at different stages of development | in-house experiment |
| (9, 15, 22 days); gebee_22 | |
| WT leaves profiling at different stages of development | in-house experiment |
| (9, 15, 22 days); gebee_9 | |
| Methyl jasmonate profiling at different time points; | in-house experiment |
| lapau_ATL-MeJA-0.5 | |
| Methyl jasmonate profiling at different time points; | in-house experiment |
| lapau_ATL-MeJA-2 | |
| Methyl jasmonate profiling at different time points; | in-house experiment |
| lapau_ATL-MeJA-6 | |
| Methyl jasmonate profiling at different time points; | in-house experiment |
| lapau_ATL-DMSO-0.5 | |
| Methyl jasmonate profiling at different time points; | in-house experiment |
| lapau_ATL-DMSO-2 | |
| Methyl jasmonate profiling at different time points; | in-house experiment |
| lapau_ATL-DMSO-6 | |
| Methyl jasmonate profiling at different time points; | in-house experiment |
| lapau_ATL-MeJA-CHX-6 | |
| DEL1 knockouts versus control (columbia) in seedlings; | in-house experiment |
| livey_Wild-type_seedling_8d | |
| DEL1 knockouts versus control (columbia) in seedlings; | in-house experiment |
| livey_DEL1_KO_seedling_8d | |
| E2F overexpression versus control (columbia WT) in | in-house experiment |
| seedlings; livey_Wild-type_seedling_8d | |
| E2F overexpression versus control (columbia WT) in | in-house experiment |
| seedlings; livey_E2Fa-DPa_OE_seedling_8d | |
| DEL1 overexpression versus control (columbia WT) in | in-house experiment |
| leaf; livey_Wild-type_leaf_15d | |
| DEL1 overexpression versus control (columbia WT) in | in-house experiment |
| leaf; livey_DEL1_OE_leaf_15d | |
| endocycle exp. In leaf - control; | in-house experiment |
| livey_Wild-type_leaf_15d | |
| endocycle exp. In leaf - DEL1 knockout; | in-house experiment |
| livey_DEL1_KO_leaf_15d | |
| endocycle exp. In leaf - CCS52A1 knockout; | in-house experiment |
| livey_CCS52A1_KO_leaf_15d | |
| endocycle exp. In leaf - CCS52B knockout; | in-house experiment |
| livey_CCS52B_KO_leaf_15d | |
| endocycle exp. In leaf—CCKB1 DN; livey_CDKB1; | in-house experiment |
| 1.DN_#9_15d | |
| endocycle exp. In leaf - KRP2 overexpression; | in-house experiment |
| livey_KRP2_OE_#7_15d | |
| endocycle exp. In leaf - DEL1 overexpression; | in-house experiment |
| livey_DEL1_OE_#2_15d | |
| WEE1 exp. - genotype columbia; livey_ROOT_TIP + | in-house experiment |
| 0 h | |
| WEE1 exp. - genotype columbia; livey_ROOT_TIP + | in-house experiment |
| 0 h_HU | |
| WEE1 exp. - genotype columbia; livey_ROOT_TIP + | in-house experiment |
| 5 h | |
| WEE1 exp. - genotype columbia; livey_ROOT_TIP + | in-house experiment |
| 5 h_HU | |
| WEE1 exp. - genotype columbia; livey_ROOT_TIP + | in-house experiment |
| 1 day | |
| WEE1 exp. - genotype columbia; livey_ROOT_TIP + | in-house experiment |
| 1 day_HU | |
| WEE1 exp. - genotype WEE1; livey_ROOT_TIP + 0 h | in-house experiment |
| WEE1 exp. - genotype WEE1; livey_ROOT_TIP + | in-house experiment |
| 0 h_HU | |
| WEE1 exp. - genotype WEE1; livey_ROOT_TIP + 5 h | in-house experiment |
| WEE1 exp. - genotype WEE1; livey_ROOT_TIP + | in-house experiment |
| 5 h_HU | |
| WEE1 exp. - genotype WEE1; livey_ROOT_TIP + | in-house experiment |
| 1 day | |
| WEE1 exp. - genotype WEE1; livey_ROOT_TIP + | in-house experiment |
| 1 day_HU | |
| lateral root initiation study WT versus mutant solitary | in-house experiment |
| root 1 (slr1) WT 0 h | |
| lateral root initiation study WT versus mutant solitary | in-house experiment |
| root 1 (slr1) WT 2 h | |
| lateral root initiation study WT versus mutant solitary | in-house experiment |
| root 1 (slr1) WT 6 h | |
| lateral root initiation study WT versus mutant solitary | in-house experiment |
| root 1 (slr1) slr1 0 h | |
| lateral root initiation study WT versus mutant solitary | in-house experiment |
| root 1 (slr1) slr1 2 h | |
| lateral root initiation study WT versus mutant solitary | in-house experiment |
| root 1 (slr1) slr1 6 h | |
| vascular tissue formation - callus formed from leaves - | DB001_ATH1_A3-Brown-cal.CEL |
| 5 days after treatment; 0.2 mgl-1 auxin(2,4-D NA | |
| IAA) 1.0 mgl-1 BA | |
| vascular tissue formation - callus formed from leaves - | DB001_ATH1_A4-Brown-cal.CEL |
| 9 days after treatment; 0.2 mgl-1 auxin(2,4-D NA | |
| IAA) 1.0 mgl-1 BA | |
| Arrested development mutant analysis - leaves - add 3 | BP001_ATH1_A1-Pickett-ADD3-1.CEL |
| Arrested development mutant analysis - leaves - control | BP001_ATH1_A3-Pickett-a1.CEL |
| Agrobacterium induced tumor vs control; Tumour tissue | RD001_ATH1_A1-deeke-tum.CEL |
| induced at the base of an inflorescence stalk with | |
| Agrobacterium tumefaciens | |
| Agrobacterium induced tumor vs control; Inflorescence | RD001_ATH1_A2-deeke-Inf.CEL |
| stalk injured at the base with a injection needle (control) | |
| Polycomb binding protein knockout vs WT; WT | KC001_ATH1_A1-Cain-WT1.CEL |
| Polycomb binding protein knockout vs WT; CDB1 | KC001_ATH1_A3-Cain-CDBI.CEL |
| knockout | |
| axillary bud dormancy_naphthalene-1-acetic acid | E-MEXP-148 |
| wild-type | |
| axillary bud dormancy_max4-1 mutant | E-MEXP-148 |
| axillary bud dormancy_wild type | E-MEXP-148 |
| MPK4 on ethylene regulation | E-MEXP-174 |
| (leaf)_wild_type_Landsberg erecta | |
| MPK4 on ethylene regulation | E-MEXP-174 |
| (leaf)_ctr1 mutant_Columbia | |
| MPK4 on ethylene regulation (leaf)_mpk4 | E-MEXP-174 |
| mutant_Landsberg erecta | |
| MPK4 on ethylene regulation (leaf)_ctr1 mpk4 double | E-MEXP-174 |
| mutant_Landsberg/Columbia | |
| systemic responses of MAPK to pathogens | E-MEXP-173 |
| (leaf)_35S:mks1_eco_Landsberg | |
| systemic responses of MAPK to pathogens (leaf)_mpk4 | E-MEXP-173 |
| mutant_eco_Landsberg | |
| systemic responses of MAPK to pathogens (leaf)_wild | E-MEXP-173 |
| type_eco_Landsberg | |
| secondary cell wall | E-MEXP-265 |
| formation_hypocotyl_eco_Landsberg | |
| secondary cell wall formation_leaf_eco_Landsberg | E-MEXP-265 |
| secondary cell wall formation_stem | E-MEXP-265 |
| base_eco_Landsberg | |
| secondary cell wall formation_stem lower | E-MEXP-265 |
| middle_eco_Landsberg | |
| secondary cell wall formation_stem tip_eco_Landsberg | E-MEXP-265 |
| secondary cell wall formation_stem upper | E-MEXP-265 |
| middle_eco_Landsberg | |
| comparison of mutants lacking CAF-1 | E-MEXP-694 |
| subunits_fas2-1_eco_Landsberg erecta | |
| comparison of mutants lacking CAF-1 | E-MEXP-694 |
| subunits_msi1-as_eco_Columbia | |
| comparison of mutants lacking CAF-1 | E-MEXP-694 |
| subunits_wild_type_eco_Columbia | |
| comparison of mutants lacking CAF-1 | E-MEXP-694 |
| subunits_wild_type_eco_Enkheim | |
| comparison of mutants lacking CAF-1 | E-MEXP-694 |
| subunits_wild_type_eco_Landsberg erecta | |
| AP2/ERF TF (bolita) (leaf)_bolita mutant | E-MEXP-809 |
| AP2/ERF TF (bolita) (leaf)_wild_type | E-MEXP-809 |
| tissue comparison_stem_greenhouse | Somerville: Tissue Type Arrays of |
| | Columbia-0_genome |
| tissue comparison_leaf_greenhouse | Somerville: Tissue Type Arrays of |
| | Columbia-0_genome |
| tissue comparison_flower_growth chamber | Somerville: Tissue Type Arrays of |
| | Columbia-0_genome |
| tissue comparison_flower_greenhouse | Somerville: Tissue Type Arrays of |
| | Columbia-0_genome |
| tissue comparison_stem_growth chamber | Somerville: Tissue Type Arrays of |
| | Columbia-0_genome |
| flower_wild-type Columbia_0 days | Weigel: Floral transition and early |
| | flower development |
| flower_wild-type Columbia_3 days | Weigel: Floral transition and early |
| | flower development |
| flower_wild-type Columbia_7 days | Weigel: Floral transition and early |
| | flower development |
| flower_wild-type Landsberg_0 days | Weigel: Floral transition and early |
| | flower development |
| flower_wild-type Landsberg_3 days | Weigel: Floral transition and early |
| | flower development |
| flower_wild-type Landsberg_5 days | Weigel: Floral transition and early |
| | flower development |
| flower_wild-type Landsberg_7 days | Weigel: Floral transition and early |
| | flower development |
| flower_mutant constans (co-2)_0 days | Weigel: Floral transition and early |
| | flower development |
| flower_mutant constans (co-2)_3 days | Weigel: Floral transition and early |
| | flower development |
| flower_mutant constans (co-2)_5 days | Weigel: Floral transition and early |
| | flower development |
| flower_mutant constans (co-2)_7 days | Weigel: Floral transition and early |
| | flower development |
| flower_mutant ft (ft-2)_0 days | Weigel: Floral transition and early |
| | flower development |
| flower_mutant ft (ft-2)_3 days | Weigel: Floral transition and early |
| | flower development |
| flower_mutant ft (ft-2)_5 days | Weigel: Floral transition and early |
| | flower development |
| flower_mutant ft (ft-2)_7 days | Weigel: Floral transition and early |
| | flower development |
| flower_mutant lfy-12 Columbia_0 days | Weigel: Floral transition and early |
| | flower development |
| flower_mutant lfy-12 Columbia_3 days | Weigel: Floral transition and early |
| | flower development |
| flower_mutant lfy-12 Columbia_5 days | Weigel: Floral transition and early |
| | flower development |
| flower_mutant lfy-12 Columbia_7 days | Weigel: Floral transition and early |
| | flower development |
| laser-capture micro-dissected_embryo cotyledon (apical) | /share/nasc/Casson_laser-capture_micro- |
| | dissected_embryonic_tissues/ |
| | CEL/KL001_ATH1_A1-Linds-cot.CEL |
| laser-capture micro-dissected_embryo root (basal) | /share/nasc/Casson_laser-capture_micro- |
| | dissected_embryonic_tissues/ |
| | CEL/KL001_ATH1_A1-Linds-roo.CEL |
| laser-capture micro-dissected_globular embryo, | /share/nasc/Casson_laser-capture_micro- |
| apical tissue | dissected_embryonic_tissues/ |
| | CEL/SC001_ATH1_A1.1-casso-gla. |
| | CEL |
| laser-capture micro-dissected_globular embryo, | /share/nasc/Casson_laser-capture_micro- |
| basal tissue | dissected_embryonic_tissues/ |
| | CEL/SC001_ATH1_A2.1-casso-glb. |
| | CEL |
| laser-capture micro-dissected_heart stage embryo, | /share/nasc/Casson_laser-capture_micro- |
| cotyledon pole (apical) | dissected_embryonic_tissues/ |
| | CEL/SC001_ATH1_A3-1-casso-hec. |
| | CEL |
| laser-capture micro-dissected_heart stage embryo, | /share/nasc/Casson_laser-capture_micro- |
| root pole (basal) | dissected_embryonic_tissues/ |
| | CEL/SC001_ATH1_A4-1-casso-her. |
| | CEL |
| water-Col0-1 hr | /share/nasc/De_Grauwe/CEL/DV001_ATH1_A2- |
| | degra-Cc1.CEL |
| water-etr1 (ethylene-insensitive mutant)-1 hr | /share/nasc/De_Grauwe/CEL/DV001_ATH1_A7- |
| | degra-Ec1.CEL |
| GA4-Col0-1 hr | /share/nasc/De_Grauwe/CEL/DV001_ATH1_A4- |
| | degra-Cg1.CEL |
| GA4-etr1 (ethylene-insensitive mutant)-1 hr | /share/nasc/De_Grauwe/CEL/DV001_ATH1_A9- |
| | degra-Eg1.CEL |
| ms1ttg-young | /share/nasc/Gema_Vizcay_Barrena/ |
| | CEL/ZW001_ATH1_A1-Wilson-mla. |
| | CEL |
| ms1ttg-old | /share/nasc/Gema_Vizcay_Barrena/ |
| | CEL/ZW002_ATH1_A2_Wilson_Rep2. |
| | CEL |
| Ler-young | /share/nasc/Gema_Vizcay_Barrena/ |
| | CEL/ZW002_ATH1_A3_Wilson_Rep2. |
| | CEL |
| Ler-old | /share/nasc/Gema_Vizcay_Barrena/ |
| | CEL/ZW001_ATH1_A4-Wilson-Ler. |
| | CEL |
| Lerttg-young | /share/nasc/Gema_Vizcay_Barrena/ |
| | CEL/ZW003_ATH1_C1-GVB_rep1. |
| | CEL |
| Lerttg-old | /share/nasc/Gema_Vizcay_Barrena/ |
| | CEL/ZW003_ATH1_C2-GVB_rep1. |
| | CEL |
| Programmed Cell Death Control | /share/nasc/Jodi_Swidzinski/CEL/JS001_ATH1_Control(3)new. |
| | CEL |
| Programmed Cell Death Heat | /share/nasc/Jodi_Swidzinski/CEL/JS001_ATH1- |
| | Heat(4).CEL |
| Programmed Cell Death Senescence | /share/nasc/Jodi_Swidzinski/CEL/JS001_ATH1_Sen(3)new. |
| | CEL |
| Wheeler-a05_SLD_SPH1 RNAi at stage 1.05, leaf | /share/nasc/Mike_Wheeler/CEL/MW001_ATH1_A1- |
| | Wheel-a05.CEL |
| Wheeler-a14_SLD_SPH1 RNAi at stage 1.14, leaf | /share/nasc/Mike_Wheeler/CEL/MW001_ATH1_A1- |
| | Wheel-a14.CEL |
| Wheeler-w05_SLD_wild type at stage 1.05, leaf | /share/nasc/Mike_Wheeler/CEL/MW001_ATH1_A1- |
| | Wheel-w05.CEL |
| Wheeler-w14_SLD_wild type at stage 1.14, leaf | /share/nasc/Mike_Wheeler/CEL/MW001_ATH1_A1- |
| | Wheel-w14.CEL |
| Ulm_1-1_4 days-345 nm | /share/nasc/Ulm_UV-B_response_of_Arabidopsis/ |
| | CEL/Ulm_1-1_4days-345nm_Rep1_ATH1. |
| | CEL |
| Ulm_1-4_4 days-305 nm | /share/nasc/Ulm_UV-B_response_of_Arabidopsis/ |
| | CEL/Ulm_1-4_4days-305m,_Rep1_ATH1. |
| | CEL |
| Ulm_1-9_4 days-345 nm, 1 hr-305 nm | /share/nasc/Ulm_UV-B_response_of_Arabidopsis/ |
| | CEL/Ulm_1-9_4days-345nm, 1hr- |
| | 305nm_Rep3_ATH1.CEL |
| Ulm_1-10_4 days-345 nm, 6 hr-305 nm | /share/nasc/Ulm_UV-B_response_of_Arabidopsis/ |
| | CEL/Ulm_1-10_4days- |
| | 345nm,6hr-305nm_Rep1_ATH1.CEL |
| microgametogenesisPollen_bicellular pollen | /share/nasc/David_Honys/CEL/DH001_ATH1_A2- |
| | BCP1.CEL |
| microgametogenesisPollen_tricellular pollen | /share/nasc/David_Honys/CEL/DH001_ATH1_A3- |
| | TCP1.CEL |
| microgametogenesisPollen_uninucleate microspores | /share/nasc/David_Honys/CEL/DH001_ATH1_A1- |
| | UNM1.CEL |
| Pourtau_1-1_lowN_leaf senescence | /share/nasc/Pourtau_sugar_acc_during_early_leaf_senescence/ |
| | CEL/Pourtau_1- |
| | 1_lowN_Rep1_ATH1.CEL |
| Pourtau_1-4_lowN-glu_leaf senescence_effect | /share/nasc/Pourtau_sugar_acc_during_early_leaf_senescence/ |
| of glucose | CEL/Pourtau_1- |
| | 4_lowN-glu_Rep1_ATH1.CEL |
| Pourtau_1-7_highN-glu_leaf senescence_effect | /share/nasc/Pourtau_sugar_acc_during_early_leaf_senescence/ |
| of nitrogen | CEL/Pourtau_1- |
| | 7_highN-glu_Rep1_ATH1.CEL |
| ozone-fumigated seedlings | /share/nasc/Short_Functional_Genomics_of_Ozone_Stress_in_Arabidopsis/ |
| | CEL/ES001_ATH1_A1-mcain-ozo. |
| | CEL |
| air-fumigated seedlings | /share/nasc/Short_Functional_Genomics_of_Ozone_Stress_in_Arabidopsis/ |
| | CEL/ES001_ATH1_A2-mcain-con. |
| | CEL |

### REFERENCES

Altschul, S. F., T. L. Madden, A. A. Schaffer, J. Zhang, Z. Zhang, W. Miller, and D. L. Lipman (1997). Gapped BLAST and PSI-BLAST: a new generation of protein database search programs, Nucleic Acids Res. 25:3389-3402.
Altschul, S. F., J. C. Wootton, E. M. Gertz, R. Agarwala, A. Morgulis, A. A. Schäffer, and K. K. Yu (2005). Protein database searches using compositionally adjusted substitution matrices, FEBS J. 272:5101-5109.
Bashir, T., N. V. Dorrello, V. Amador, D. Guardavaccaro, and M. Pagano, Nature 428:190 (Mar. 11, 2004).
Brukhin, V., J. Gheyselinck, V. Gagliardini, P. Genschik, and U. Grossniklaus, The Plant cell 17:2723 (October 2005).
Camasses, A., A. Bogdanova, A. Shevchenko, and W. Zachariae, Mol. Cell. 12:87 (July 2003).
Capron, A., L. Okresz, and P. Genschik, Trends in plant science 8:83 (February 2003).
Churchman, M. L. et al., The Plant cell 18:3145 (November 2006).
Clough, S. J. and A. F. Bent (1998). Floral dip: a simplified method for Agrobacterium-mediated transformation of Arabidopsis thaliana Plant J. 16:735-743.
Cui. J. et al., Nucleic acids research 36:D999 (January 2008).
De Veylder, L., G. Segers, N. Glab, M. Van Montagu, and D. Inze, Journal of experimental botany 48:2113 (December 1997).
Erhardt, M. et al., J. Cell. Sci. 115:2423 (Jun. 1, 2002).
Forment, J., M. A. Naranjo, M. Roldan, R. Serrano, and O. Vicente, Plant J. 30:511 (June 2002).
Geisler-Lee, J. et al., Plant Physiol. 145:317 (October 2007).
Hase, Y., K. H. Trung, T. Matsunaga, and A. Tanaka, Plant J. 46:317 (April 2006).
Hayes, M. J. et al., Nat. Cell Biol. 8:607 (June 2006).
Huala, E. et al., Nucleic acids research 29:102 (Jan. 1, 2001).
Huanca-Mamani, W., M. Garcia-Aguilar, G. Leon-Martinez, U. Grossniklaus, and J. P. Vielle-Calzada, Proc. Natl. Acad. Sci. U.S.A. 102:17231 (Nov. 22, 2005).
Ishida, S. et al., Mol. Cell Biol. 21:4684 (July 2001).
Jensen, L. J., T. S. Jensen, U. de Lichtenberg, S. Brunak, and P. Bork, Nature 443:594 (Oct. 5, 2006).
Kerrien S. et al., Nucleic acids research 35:D561 (January 2007).
Kwon, C. S. et al., Development 133:3223 (August 2006).
Maere, S., K. Heymans, and M. Kuiper, Bioinformatics (Oxford, England) 21:3448 (Aug. 15, 2005).
Magyar, Z. et al., The Plant cell 17:2527 (September 2005).
Mayer, M. L., S. P. Gygi, R. Aebersold, and P. Hieter, Molecular cell 7:959 (May, 2001).
Menges, M., L. Hennig, W. Gruissem, and J. A. Murray, Plant molecular biology 53:423 (November 2003).
Menges, M., S. M. de Jager, W. Gruissem, and J. A. Murray, Plant J. 41:546 (February 2005).
Muller S. et al., Curr. Biol. 14:412 (Mar. 9, 2004).
Peres A. et al., J. Biol. Chem. 282:25588 (Aug. 31, 2007).
Popescu, S. C. et al., Proc. Natl. Acad. Sci. U.S.A. 104:4730 (Mar. 13, 2007).
Rohila, J. S. et al., Plant J. 46:1 (April 2006).
Shultz, R. W., V. M. Tatineni, L. Hanley-Bowdoin, and W. F. Thompson, Plant Physiol. 144:1697 (August 2007).
Seifert, G. J., Curr. Opin. Plant Biol. 7:277 (June 2004).
Shannon P. et al., Genome Res. 13:2498 (November 2003).
Takahashi, N. et al., The EMBO journal 27:1840 (Jul. 9, 2008).
Tsesmetzis, N. et al., The Plant cell 20:1426 (June 2008).
Vandepoele, K. et al., The Plant cell 14:903 (April 2002).
Vandepoele, K. et al., Plant Physiol. 139:316 (September 2005).
Vandepoele, K., T. Casneuf, and Y. Van de Peer, Genome Biol. 7:R103 (2006).
Van Leene, J. et al., Mol. Cell Proteomics 6:1226 (July 2007).
Van Leene, J., E. Witters, D. Inze, and G. De Jaeger, Trends in plant science 13:517 (Sep. 2, 2008).
Vastrik, I. et al., Genome biology 8:R39 (2007).
Vodermaier, H. C., C. Gieffers, S. Maurer-Stroh, F. Eisenhaber, and J. M. Peters, Curr. Biol. 13:1459 (Sep. 2, 2003).
Wuarin, J., V. Buck, P. Nurse, and J. B. Millar, Cell 111:419 (Nov. 1, 2002).

1. A method of isolating cell-cycle related proteins, the method comprising;
performing a tap analysis using utilizing a known cell cycle protein as bait to produce results,
correcting the results for non-specific interactions to produce corrected results, and
reconfirming the corrected results.

2. A method of modulating plant growth, the method comprising:
identifying a cell-cycle related protein with the method of claim 1,
utilizing the identified cell-cycle related protein to modulate plant growth.

3. The method according to claim 2, wherein said cell-cycle related protein is selected from the group consisting of At1g56110, At3g17020, At3g21140, At5g25460, At5g60790, At4g38900, At3g49240, At5g24690, At1g06070, At4g34150, At1g20480, At5g20920, At3g15970, At5g13030, At1g01880, At5g07310, At2g46610, At1g10690, At3g04710, At3g24690, At4g16130, At2g05830, At1g29220, At1g55890, At1g60650, At1g70830, At2g43140, At1g77180, At5g18620, At5g02530, At5g14170, At1g52730, At2g33340, At1g03060, At3g62240, At4g38740, At5g61220, At3g53880, At3g56860, At1g01970, At1g19520, At1g14620, At2g03820, At3g01280, At3g56690, At5g41190, At5g03740, At1g42440, At2g28450, At1g09760, At1g10840, At3g11830, At5g54900, At1g31760, At1g61870, At3g11760, At1g05805, At1g29200, At4g13850, At4g38780, At1g71380, At3g13640, At5g25060, At1g43700, At2g46020, At3g55760, At5g21160, and a variant of any thereof.

4. A method of promoting plant growth, the method comprising
utilizing a protein selected from the group consisting of At1g56110, At3g17020, At3g21140, At5g25460, At5g60790, or a variant thereof, to promote plant growth.

5. The method according to claim 4, wherein said utilizing is overexpression.

6. The method according to claim 4, wherein promotion of plant growth is measured as plant biomass increase.

7. The method according to claim 2, wherein said plant is a crop plant.

8. The method according to claim 7, wherein said crop plant is a cereal.

9. The method according to claim 8, wherein said cereal is selected from the group consisting of rice, maize, wheat, barley, millet, rye, sorghum and oats.

10. A cell cycle related protein isolated with the method of claim 1.

11. The method according to claim 5, wherein promotion of plant growth is measured as plant biomass increase.

12. The method according to claim 3, wherein the plant is a crop plant.

13. The method according to claim 12, wherein the crop plant is a cereal.

14. The method according to claim 13, wherein the cereal is selected from the group consisting of rice, maize, wheat, barley, millet, rye, sorghum and oats.

15. The method according to claim 4, wherein the plant is a crop plant.

16. The method according to claim 5, wherein the plant is a crop plant.

17. The method according to claim 6, wherein the plant is a crop plant.

18. The method according to claim 17, wherein the crop plant is a cereal.

19. The method according to claim 18, wherein the cereal is selected from the group consisting of rice, maize, wheat, barley, millet, rye, sorghum and oats.

20. A method of promoting growth of a plant, wherein the plant is selected from the group consisting of rice, maize, wheat, barley, millet, rye, sorghum, and oat, the method comprising:
overexpressing a protein in the plant, said plant selected from the group consisting of At1g56110, At3g17020, At3g21140, At5g25460, and At5g60790,
so as to promote the plant's growth.


SEQUENCE LISTING (5 sequences)

SEQ ID NO: 1 - protein, 432 aa, Arabidopsis thaliana
MAMYVIYESSSGYGLFEVHGLDEIGQNTEAVRTSVSDLSRFGRVVQLTAFHPFESALDALNQVNAVSEGVMTDELRSFLELNLPKVKEGKKPKFSLGLAEPKLGSHIFEATKIPCQSNEFVLELLRVVRQHFDRFIKDLKPGDLEKSQLGLAHSYSRAKVKFNVNRVDNMVIQAIFMLDTLDKDINSFAMRVREWYSWHFPELVKIVNDNYLYARVSKMIDDKSKLTEDHIPMLTEVLGDEDKAKEVIEAGKASMGSDLSPLDLINVQTFAQKVMDLADYRKKLYDYLVTKMSDIAPNLAALIGEMVGARLISHAGSLTNLAKCPSSTLQILGAEKALFRALKTRGNTPKYGLIFHSSFIGRASAKNKGRIARYLANKCSIASRIDCFADGATTAFGEKLREQVEERLEFYDKGVAPRKNVDVMKEVIENLK

SEQ ID NO: 2 - protein, 163 aa, Arabidopsis thaliana
MAESGGRRIGVAVDFSDCSKKALSWAIDNVVRDGDHLILITIAHDMNYEEGEMQLWETVGSPFIPMSEFSDAAVMKKYALKPDAETLDIVNTAARKKTITVVMKIYWGDPREKICAAAEQIPLSSLVMGNRGLGGLKRMIMGSVSNHVVNNVACPVTVVKAHI

SEQ ID NO: 3 - protein, 387 aa, Arabidopsis thaliana
MEALFTSTHTPNLQTKPLLKSPLPTSSQSSCWFCNSLPKTQFPKLRLSNGSSHGLRIQALLRNETPSEGEDNNGFGFFPGDIFSLSQEKLGSNSNGETSHSVIDVEASLAHPQGGGGNRAGLFRTPISGGVQNATSAHALPRPALAVRNLLEQARFAHLCTVMSKMHHRREGYPFGSLVDFAPDRMGHPIFLFSPLAIHTRNLLNEPRCSLVVQIPGWSGLSNARVTLFGDVYPLSEDEQEWAHKQYIAKHPHGPSEQWGNFHYFRMQNISDIYFIGGFGTVAWVDVKEYEGLQPDKIAVDGGERNLKELNAIFSKPLRELLSTESEVDDAALISIDSKGIDVRVRQGAQFNIQRLAFEEGHGVETLEEAKSALWKVLEKVKLNLQK

SEQ ID NO: 4 - protein, 186 aa, Arabidopsis thaliana
MRSLYPPRPTNKNILKNGGFEEGPLVLPGSTTGVLIPPFIEDDHSPLPGWMVESLKAVKYVDVEHFSVPQGRRAIELVAGKESAIAQVVRTVIGKTYVLSFAVGDANNACKGSMVVEAFAGKDTLKVPYESKGTGGFKRASIRFVAVSTRSRIMFYSTFYAMRSDDFSSLCGPVIDDVKLISVRKP

SEQ ID NO: 5 - protein, 237 aa, Arabidopsis thaliana
IARDSVLVFRFADVGKLPPPVLQFVEVSFGYTPDYLIYKNIDFGVDLDSRVALVGPNGAGKSTLLKLMTGELHPTEGMVRRHNHLKIAQYHQHLAEKLDLELPALLYMMREFPGTEEEKMRAAIGRFGLTGKAQVMPMKNLSDGQRSRVIFAWLAYKQPNMLLLDEPTNHLDIETIDSLAEALNEWDGGLVLVSHDFRLINQVAHEIWVCEKQCITKWNGDIMDFKRHLKAKAGLED